(19) United States
(12) Patent Application Publication
(10) Pub. No.: US 2024/0245284 A1
(43) Pub. Date: Jul. 25, 2024

(54) Steerable instrument for endoscopic or invasive applications

(71) Applicant: Fortimedix Assets II B.V., Geleen (NL)

(72) Inventor: Mattheus Hendrik Louis Thissen, Swalmen (NL)

(73) Assignee: Fortimedix Assets II B.V., Geleen (NL)

(21) Appl. No.: 18/566,111

(22) PCT Filed: Jun. 8, 2022
(86) PCT No.: PCT/NL2022/050318

§ 371 Date: Dec. 1, 2023

(30) Foreign Application Priority Data
Jun. 8, 2021 (NL) 2028414
Dec. 16, 2021 (NL) 2030158

(51) Int. Cl. A61B 1/005; A61B 1/00; A61M 25/00; A61M 25/01

(52) U.S. Cl. CPC A61B 1/0057; A61B 1/00057; A61B 1/0011; A61M 25/0013; A61M 25/0138; A61M 25/0147; A61M 2205/3327

(57) ABSTRACT

A cylindrical instrument has a first tube (1601; 1619), a second tube (1620) surrounding the first tube (1601; 1619), and a third tube (1602; 1621) surrounding the second tube (1620). The instrument has a deflectable tip section (1613), a steering section (1618), a flexible body section (1615) between the tip section (1613) and the steering section (1618), a length compensation section (1617), and one or more steering wires (16(i)) extending from the steering section (1618) to the tip section (1613) such that the tip section (1613) can be deflected by moving the one or more steering wires (16(i)) in a longitudinal direction of the cylindrical instrument. The cylindrical instrument has a Bowden cable arrangement for each steering wire (16(i)) inside the length compensation section (1617), each Bowden cable arrangement has a steering wire (16(i)) surrounded by steering wire guiding portions. The steering wires (16(i)) and the steering wire guiding portions are portions of the first tube (1619), the second tube (1620), or the third tube (1621).

### FIELD OF THE INVENTION

The present invention relates to a steerable instrument for endoscopic and/or invasive type of applications, such as in surgery. The steerable instrument according to the invention can be used in both medical and non-medical applications. Examples of the latter include inspection and/or repair of mechanical and/or electronic hardware at locations that are difficult to reach. Hence, terms used in the following description such as endoscopic application or invasive instrument, must be interpreted in a broad manner.

### BACKGROUND ART

Transformation of surgical interventions that require large incisions for exposing a target area into minimal invasive surgical interventions, i.e. requiring only natural orifices or small incisions for establishing access to the target area, is a well-known and ongoing process. In performing minimal invasive surgical interventions, an operator such as a physician, requires an access device that is arranged for introducing and guiding invasive instruments into the human or animal body via an access port of that body. In order to reduce scar tissue formation and pain to a human or animal patient, the access port is preferably provided by a single small incision in the skin and underlying tissue. In some applications, a natural orifice of the body can be used as an entrance. Furthermore, the access device preferably enables the operator to control one or more degrees of freedom that the invasive instruments offer. In this way, the operator can perform required actions at the target area in the human or animal body in an ergonomic and accurate manner with a reduced risk of clashing of the instruments used.

Surgical invasive instruments and endoscopes are well-known in the art. Both the invasive instruments and endoscopes can comprise a steerable tube that enhances its navigation and steering capabilities. Such a steerable tube may comprise a proximal end part including at least one flexible zone, a distal end part including at least one flexible zone, and an intermediate part, wherein the steerable tube further comprises a steering arrangement that is adapted for translating a deflection of at least a part of the proximal end part relative to the intermediate part into a related deflection of at least a part of the distal end part. Alternatively, the distal flexible zone may be steered by a robotic instrument arranged at the proximal end of the steerable instrument.

Steerable invasive instruments may comprise a handle that is arranged at the proximal end part of the steerable tube for steering the tube and/or for manipulating a tool that is arranged at the distal end part of the steerable tube. Such a tool can for example be a camera, a manual manipulator, e.g. a pair of scissors, forceps, or manipulators using an energy source, e.g. an electrical, ultrasonic or optical energy source.

Furthermore, such a steerable tube may comprise a number of co-axially arranged cylindrical elements including an outer cylindrical element, an inner cylindrical element and one or more intermediate cylindrical elements depending on the number of flexible zones in the proximal and distal end parts of the tube and the desired implementation of the steering members of the steering arrangement, i.e. all steering members can be arranged in a single intermediate cylindrical element or the steering members are divided in different sets and each set of steering members is arranged, at least in part, in a different or the same intermediate cylindrical element.

In most prior art devices, the steering arrangement comprises conventional steering cables with, for instance, sub 1 mm diameters as steering members, wherein the steering cables are arranged between related flexible zones at the proximal and distal end parts of the tube. Other steering units at the proximal end, like ball shaped steering units or robot driven steering units, may be applied instead.

However, as steering cables have many well-known disadvantages, for some applications one may want to avoid them and to implement the steering members by one or more sets of steering wires that form integral parts of the one or more intermediate cylindrical elements. Each of the intermediate cylindrical elements including the steering wires can be fabricated either by using a suitable material addition technique, such as injection molding or plating, or by starting from a tube and then using a suitable material removal technique, such as laser cutting, photochemical etching, deep pressing, conventional chipping techniques such as drilling or milling or high-pressure water jet cutting systems. Steering wires manufactured in that way are, then, implemented as longitudinal strips resulting from the tube material, and can be used as pulling/pushing wires. Of the aforementioned material removal techniques, laser cutting is very advantageous as it allows a very accurate and clean removal of material under reasonable economic conditions.

The inner and outer cylindrical elements may be manufactured from tubes too. These tubes should be flexible at locations where the distal end, and possibly the proximal end too, of the instrument is bendable. Also at other locations where the instrument should be flexible, the inner and outer cylindrical elements should be flexible. This can be implemented by providing the inner and outer cylindrical elements with hinges at these flexible locations. Such hinges may result from (laser) cutting predetermined patterns in the tube. Many different patterns are known from the prior art. Which pattern to use depends on design requirements at the location concerned including but not limited to the required bending angle, bending flexibility, longitudinal stiffness, and radial stiffness.

Further details regarding the design and fabrication of the abovementioned steerable tube and the steering arrangement thereof have been described for example in WO 2009/112060 A1, WO 2009/127236 A1, U.S. Pat No. 13/160,949, and U.S. Pat. No. 13/548,935 of the applicant, all of which are hereby incorporated by reference in their entirety.

As is known from for example a flexible endoscopic instrument with a steerable tip, flexible invasive steerable instruments can show performance flaws with respect to steerable tip control. When such a flexible instrument is inserted into a body through a curved channel, either an endoscope or a natural body lumen, bending of the instrument causes displacement of the longitudinal tip steering elements. Because in conventionally built instruments the steering elements, e.g. wires, are fixed to a steering device, like a handle, at the proximal side and to the steerable tip at the distal side, movement of the steering wires will result in deflection of the steering device and or deflection of the steerable tip. This causes the problem that when the instrument is advanced through a narrow curved channel, and when one holds the steering device in a fixed position, the tip will deflect uncontrollable during advancement and can either lock up in, for example, a narrow endoscope working channel or it can damage tissue in for example a soft tissue natural body lumen like the lung bronchi or the esophagus.

Another problem is that when the instrument passed the entrance channel and the instrument tip reached the targeted operation site, the tip deflection does not match the steering device deflection anymore. So a neutral position of the steering device does not result in a neutral position of the steerable tip. This offset does adversely affect eye-hand coordination of the user.

Yet another problem with flexible steerable instruments is that when the tip is steered with the steering elements, also the body will be steered by the steering elements because the whole body, mechanically, behaves like a steerable tip. The ratio of deflection between the body and tip deflection merely depends on the bending stiffness of the body and the tip. The stiffer the body is with respect to the stiffness of the tip, the more the tip will be steered. In practice, the tip is more flexible than the body, but still there is a tendency that steering the tip will result also in body deflection which on its turn will result in side forces on the surrounding channel that tends to keep the instrument body in a certain curvature. If the surrounding channel exists of soft body tissue, this is a strongly unwanted instrument behaviour, since the side forces might damage the surrounding tissue. Also body movement might disturb the positioning of the steerable tip at the target site and makes accurate and predictable tip steering more difficult.

A partial solution to this problem that addresses the problem of unwanted tip steering due to bending of the instrument body is described in WO2014/011049. This solution describes an instrument in which the steering wires can be de-coupled from the steering device and the ends of these steering wires and hence the instrument tip can move freely when the instrument is advanced through a curved entrance path. Once the instrument tip passed the entrance channel and is at the targeted operation site, the steering wires are re-coupled to the steering device and the instrument tip can now be steered. The disadvantages of this solution are that the instrument is mechanically more complex and requires more parts to build. Another disadvantage is that the operator has to follow a certain procedure for passing the curved entrance channel with which he can make mistakes or which he might forget to perform. Yet another disadvantage is that the problem of body steering (side forces) is still not addressed.

Prior art solutions have in common that they are built from specially fabricated tubings, coils and machined parts and that assembly of such instruments is usually a time consuming and difficult process. Also tolerances of the separate parts add up in the assembly and can be the cause of a wide spread in for example instrument performance, often requiring an individual calibration of each instrument.

### SUMMARY OF THE INVENTION

It is an object of the invention to provide a steerable instrument for endoscopic and/or invasive type of applications where at least one of the above mentioned problems are solved or at least reduced.

To that end, independent aspects of the invention are defined in independent claims whereas dependent claims relate to advantageous embodiments.

Some embodiments comprise a Bowden cable arrangement.

In this application, the terms “proximal” and “distal” are defined with respect to an operator, e.g. a robot or physician that operates the instrument or endoscope. For example, a proximal end part is to be construed as a part that is located near the robot or physician and a distal end part as a part located at a distance from the robot or physician, i.e., in the area of operation.

In many embodiment, the invention comprises an instrument having the same and improved performance as prior solutions, but which is built with significantly less separate parts and significantly less assembly effort. All the necessary elements to construct a steerable instrument, including a Bowden-cable construction may be integrally manufactured, in a largely pre-assembled state, from a number of tubes. The only remaining assembly steps consist of sliding the tubes into each other and attach the tubings to each other in the required places. The preassembled parts can be made in a tube wall by material deposition processes like 3D printing or plating techniques. Preferably the preassembled parts can be made by material removal processes from a solid wall metal or plastic tube (stainless steel, cobalt chromium alloys, super-elastic alloys like nitinol, etc). The material removal processes that can be used are for example conventional chipping processes, water jet cutting, etching and preferably laser cutting processes.

Therefore, those embodiments of this invention enable a significant reduction of manufacturing costs of such instruments and therefor the costs of an intervention in which these instruments are used. It even becomes commercially viable to use these instruments only once, and then throw them away. This increases the safety of an intervention because one can now use new instruments instead of pre-used and re-sterilized instruments that are known to have a 10% risk of post procedural complication due to contaminating or infecting the patient with not properly cleaned or re-sterilized pre-used instruments.

Another advantage of such an instrument is that by using this integrated way of producing parts in a pre-assembled state, that they always fit to each other and that minimal play between the parts can be achieved. This is especially true when a laser cutting process is used. The minimal achievable play between two integrally manufactured parts is as low as the width of the used laser beam, which can be as small as 0.01 mm. Typically a play of 0.01 to 0.05 mm can be obtained easily. The integral fabrication of parts according to the invention therefor is so accurate with respect to fitting of parts and the play between them, that an improved accuracy and repeatability of the instrument's functional performance is ensured.

### BRIEF DESCRIPTION OF THE DRAWINGS

Further features and advantages of the invention will become apparent from the description of the invention by way of non-limiting and non-exclusive embodiments. These embodiments are not to be construed as limiting the scope of protection. The person skilled in the art will realize that other alternatives and equivalent embodiments of the invention can be conceived and reduced to practice without departing from the scope of the present invention. Embodiments of the invention will be described with reference to the figures of the accompanying drawings, in which like or same reference symbols denote like, same or corresponding parts, and in which:

FIG. 1 shows a schematic cross sectional view of an invasive instrument assembly having one bendable distal end portion and one proximal end portion which controls the bending of the bendable distal end portion by means of strips cut out in a cylindrical element.

FIG. 2 shows a schematic overview of three cylindrical elements from which the instrument of FIG. 1 may be manufactured.

FIG. 3a shows a portion of an intermediate cylindrical element of the instrument of FIGS. 1 and 2.

FIG. 3b shows an alternative example of an intermediate cylindrical element of such an instrument.

FIG. 4 shows an example intermediate cylindrical element and an inner cylindrical element inserted in the intermediate cylindrical element.

FIG. 5 shows an outside view of a steerable invasive instrument assemble having two steerable bendable distal end portions and two proximal flexible control portions.

FIG. 6 shows an enlarged view of the distal tip of the instrument shown in FIG. 5.

FIG. 7 shows a cross section view through the invasive instrument shown in FIG. 5.

FIGS. 8 and 9 show examples of how the invasive instrument of FIGS. 5 and 7 can bend.

FIG. 10 shows an alternative embodiment of the invasive instrument shown in FIGS. 5-9, wherein at least a portion of an intermediate section between the distal end and the proximal end is flexible too.

FIGS. 11 and 12 show schematic examples of using an invasive instrument as an endoscopic surgical instrument in which the intermediate section between the distal end and the proximal end is flexible too such that the invasive instrument can be inserted in a natural body canal like the intestinal canal, and the oesophagus.

FIGS. 13-15 show prior art instruments with Bowden cable arrangements to compensate steering wire length changes in the body section when the body section bends.

FIGS. 16-25B, 29 and 30 show instruments with a Bowden cable arrangement in which both steering wires and steering wire guiding elements are manufactured as portions from tubes surrounding one another. In these figures, the Bowden cable arrangements extend radially from a central axis of the instrument. FIG. 29 shows a case wherein the Bowden cable arrangement extends radially inward whereas in all other examples it extends radially outward.

FIGS. 26A-26F show an embodiment in which the Bowden cable arrangement extends in a spiral fashion in the length compensation section.

FIG. 27 shows a Bowden cable arrangement in which both steering wires and steering wire guiding elements are manufactured as portions from a single tube and are configured such that length compensation movements occur in the tangential direction of the tube.

FIGS. 28a, 28b show an embodiment in which portions of steering wires inside the length compensation section as manufactured from a tube are designed such that they can mechanically compensate steering wire length changes in the body section when the body section bends.

FIGS. 31a-31c show an embodiment in which the length compensation section is implemented by changing the position of the portions of steering wires and steering wire guiding portions inside the length compensation section in the longitudinal direction as controlled by a processor. They also show reaction force compensation.

FIGS. 32-42H show more embodiments of steerable instruments with a length compensation mechanism.

FIGS. 43-45 show examples of fracture elements between two adjacent portions of a tube.

FIGS. 46-49 show examples of melt elements between two adjacent portions of a tube.

### DESCRIPTION OF EMBODIMENTS

For the purpose of the present document, the terms cylindrical element and tube may be used interchangeably, i.e., like the term tube a cylindrical element also refers to a physical entity. The invention will be explained with reference to steering wires which are cut from such cylindrical elements and are operative as push and/or pull steering wires to transfer movement of the steering wires at the proximal end of the instrument to the distal end to thereby control bending of one or more flexible distal end portions. However, in some embodiments, the invention can also be implemented with steering wires made in a classic way and not resulting from cutting them out of a tube. In some embodiments, steering wire guiding element portions are also made by cutting them out of one or more tubes. They sense or measure longitudinal length differences of the instrument body walls by means of generating a length displacement at one of their ends.

Instruments in which the Invention can be Applied

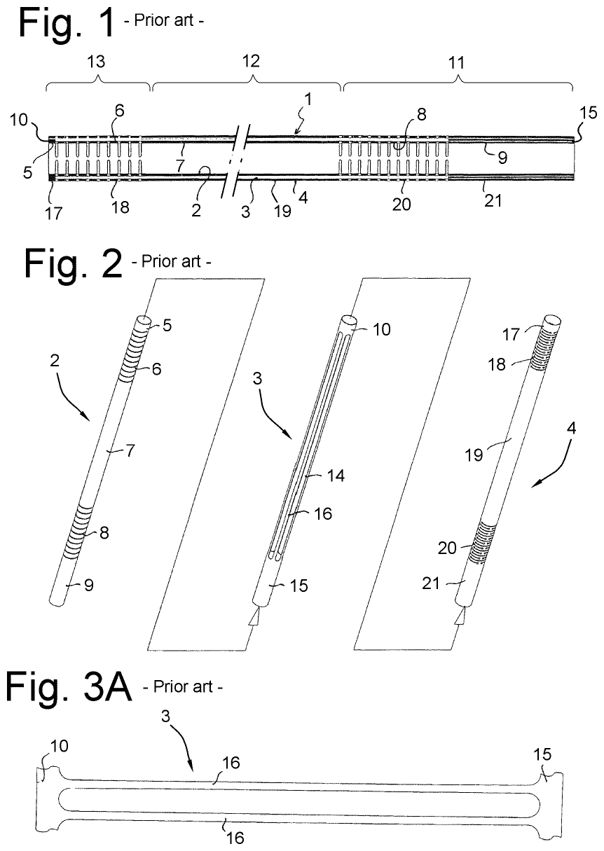

FIGS. 1, 2, 3a, and 3b are known from WO2009/112060. They are explained in detail because the present invention can be applied in this type of instruments.

FIG. 1 shows a longitudinal cross-section of a prior art steerable instrument comprising three co-axially arranged cylindrical elements, i.e. inner cylindrical element 2, intermediate cylindrical element 3 and outer cylindrical element 4. Suitable materials to be used for making the cylindrical elements 2, 3, and 4 include stainless steel, cobalt-chromium, shape memory alloy such as Nitinol®, plastic, polymer, composites or other materials that can be shaped by material removal processes like laser cutting or EDM. Alternatively, the cylindrical elements can be made by a 3D printing process or other known material deposition processes.

The inner cylindrical element 2 comprises a first rigid end part 5, which is located at a distal end part 13 of the instrument, a first flexible part 6, an intermediate rigid part 7 located at an intermediate part 12 of the instrument, a second flexible part 8 and a second rigid end part 9, which is located at a proximal end part 11 of the instrument.

The outer cylindrical element 4 also comprises a first rigid end part 17, a first flexible part 18, an intermediate rigid part 19, a second flexible part 20 and a second rigid end part 21. The lengths of the parts 5, 6, 7, 8, and 9, respectively, of the cylindrical element 2 and the parts 17, 18, 19, 20, and 21, respectively, of the cylindrical element 4 are, preferably, substantially the same so that when the inner cylindrical element 2 is inserted into the outer cylindrical element 4, these different respective parts are longitudinally aligned with each other.

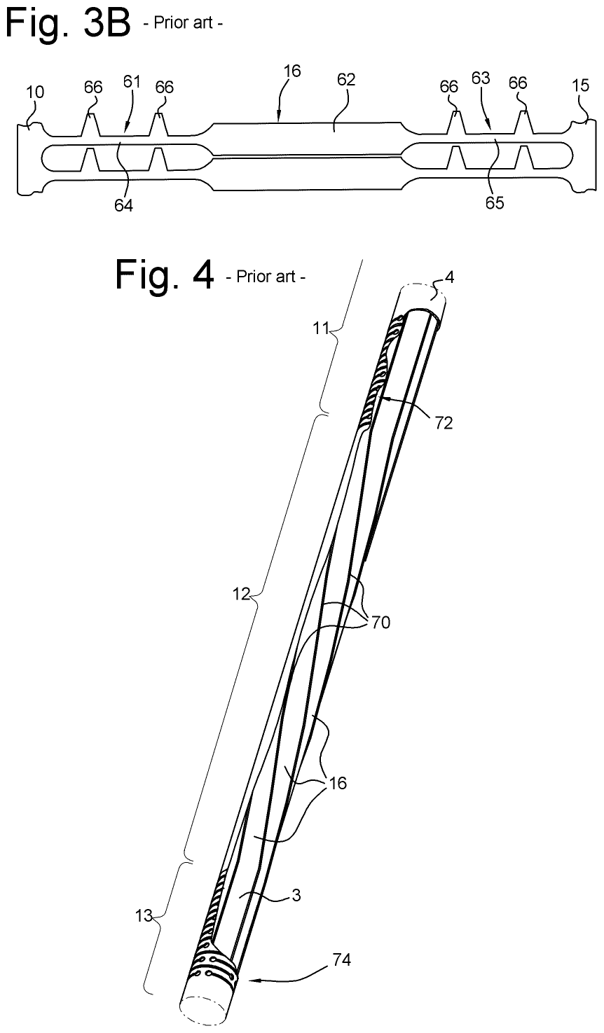

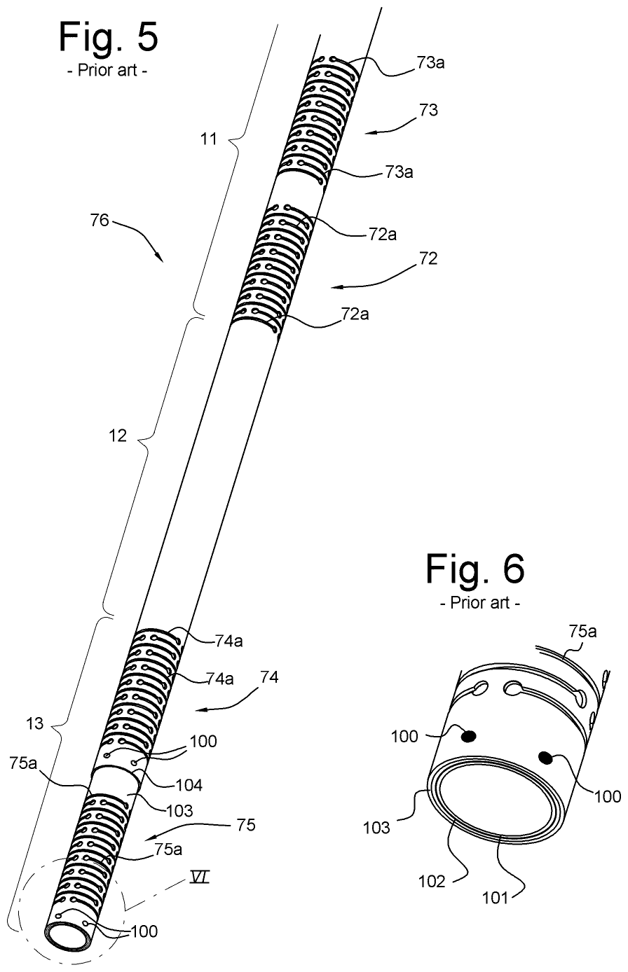

The intermediate cylindrical element 3 also has a first rigid end part 10 and a second rigid end part 15 which in the assembled condition are located between the corresponding rigid parts 5, 17 and 9, 21 respectively of the two other cylindrical elements 2, 4. The intermediate part 14 of the intermediate cylindrical element 3 comprises one or more separate steering wires 16 which can have different forms and shapes as will be explained below. They are made from the cylindrical element 3 themselves and have the form of a longitudinal strip. In FIG. 3a, three such steering wires 16 are shown. After assembly of the three cylindrical elements 2, 3 and 4 whereby the element 2 is inserted in the element 3 and the two combined elements 2, 3 are inserted into the element 4 (any other order is possible), at least the first rigid end part 5 of the inner cylindrical element 2, the first rigid end part 10 of the intermediate cylindrical element 3 and the first rigid end part 17 of the outer cylindrical element 4 at the distal end of the instrument are attached to each other, e.g., by means of glue or one or more laser welding spots. In the embodiment shown in FIGS. 1 and 2, also the second rigid end part 9 of the inner cylindrical element 2, the second rigid end part 15 of the intermediate cylindrical element 3 and the second rigid end part 21 of the outer cylindrical element 4 at the proximal end of the instrument are attached to each other, e.g. by means of glue or one or more laser welding spots, such that the three cylindrical elements 2, 3, 4 form one integral unit.

In the embodiment shown in FIG. 2 the intermediate part 14 of intermediate cylindrical element 3 comprises a number of steering wires 16 with a uniform cross-section so that the intermediate part 14 has the general shape and form as shown in the unrolled condition of the intermediate cylindrical element 3 in FIG. 3a. From FIG. 3a it also becomes clear that the intermediate part 14 is formed by a number of over the circumference of the intermediate cylindrical part 3, possibly equally, spaced parallel steering wires 16. Advantageously, the number of steering wires 16 is at least three, so that the instrument becomes fully controllable in any direction, but any higher number is possible as well. The number of steering wires 16 may, e.g., be six or eight.

It is observed that the steering wires 16 need not have a uniform cross section across their entire length. They may have a varying width along their length, possibly such that at one or more locations adjacent steering wires 16 are only separated by a small slot resulting from the laser cutting in the cylindrical element 3. These wider portions of the steering wires, then, operate as spacers to prevent adjacent steering wires 16 from buckling in a tangential direction in a pushed state. Spacers may, alternatively, be implemented in other ways.

An embodiment with spacers is shown in FIG. 3b which shows two adjacent steering wires 16 in an unrolled condition. In the embodiment shown in FIG. 3b each steering wire 16 is composed of three portions 61, 62 and 63, co-existing with the first flexible part 6, 18 the intermediate rigid part 7, 19 and the second flexible part 8, 20 respectively. In the portion 62 coinciding with the intermediate rigid portion, each pair of adjacent steering wires 16 is almost touching each other in the tangential direction so that in fact only a narrow slot is present there between just sufficient to allow independent movement of each steering wire. The slot results from the manufacturing process and its width is, e.g., caused by the diameter of a laser beam cutting the slot.

In the other two portions 61 and 63 each steering wire consists of a relatively small and flexible part 64, 65 as seen in circumferential direction, so that there is a substantial gap between each pair of adjacent flexible parts, and each flexible part 64, 65 is provided with a number of spacers 66, extending in the tangential direction and almost bridging completely the gap to the adjacent flexible part 64, 65. Because of these spacers 66 the tendency of the steering wires 16 in the flexible portions of the instrument to shift in tangential direction is suppressed and tangential direction control is improved. The exact shape of these spacers 66 is not very critical, provided they do not compromise flexibility of flexible parts 64 and 65. The spacers 66 may form an integral part with the flexible parts 64, 65 and may result from a suitable laser cutting process too.

In the embodiment shown in FIG. 3b the spacers 66 are extending towards one tangential direction as seen from the flexible part 64, 65 to which they are attached. It is however also possible to have these spacers 66 extending to both circumferential directions starting from one flexible part 64, 65. By using this it is either possible to have alternating types of flexible parts 64, 65 as seen along the tangential direction, wherein a first type is provided at both sides with spacers 66 extending until the next flexible part, and a second intermediate set of flexible parts 64, 65 without spacers 66. Otherwise it is possible to have flexible parts with cams at both sides, where as seen along the longitudinal direction of the instrument the cams originating from one flexible part are alternating with spacers originating from the adjacent flexible parts. It is obvious that numerous alternatives are available.

The production of such an intermediate part is most conveniently done by injection moulding or plating techniques or starting from a cylindrical tube with the desired inner and outer diameters and removing parts of the wall of the cylindrical tube required e.g. by laser or water cutting to end up with the desired shape of the intermediate cylindrical element 3. However, alternatively, any 3D printing method can be used.

The removal of material can be done by means of different techniques such as laser cutting, photochemical etching, deep pressing, conventional chipping techniques such as drilling or milling, high pressure water jet cutting systems or any suitable material removing process available. Preferably, laser cutting is used as this allows for a very accurate and clean removal of material under reasonable economic conditions. The above mentioned processes are convenient ways as the cylindrical element 3 can be made so to say in one process, without requiring additional steps for connecting the different parts of the intermediate cylindrical element as required in the conventional instruments, where conventional steering cables must be connected in some way to the end parts. The same type of technology can be used for producing the inner and outer cylindrical elements 2 and 4 with their respective flexible parts 6, 8, 18 and 20. These flexible parts 6, 8, 18 and 20 can be manufactured as hinges resulting from cutting out any desired pattern from the cylindrical elements, e.g., by using any of the methods described in European patent application 08 004 373.0 filed on 10.03.2008, page 5, lines 15-26, but any other suitable process can be used to make flexible portions.

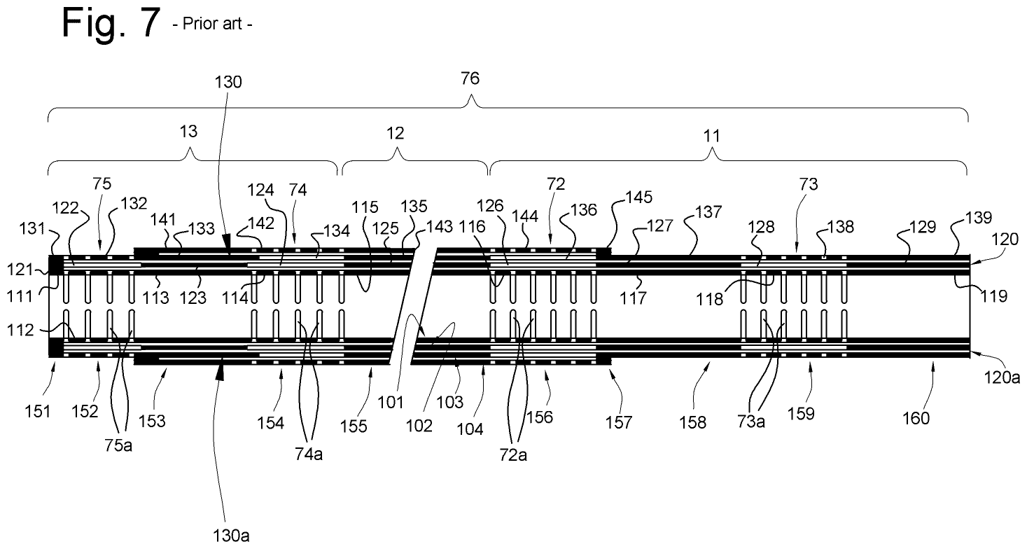

It is observed that the instruments shown in FIGS. 4-10 are known from prior art WO2020/214027. Also in these instruments the present invention can be applied.

FIG. 4 shows an exemplary embodiment of longitudinal (steering) elements 16 that have been obtained after providing longitudinal slots 70 to the wall of the intermediate cylindrical element 3 that interconnects proximal flexible zone 14 and distal flexible zone 16 as described above. Here, steering wires 16 are, at least in part, spiralling about a longitudinal axis of the instrument such that an end portion of a respective steering element 16 at the proximal portion of the instrument is arranged at another angular orientation about the longitudinal axis than an end portion of the same steering wire 16 at the distal portion of the instrument. Were the steering wires 16 arranged in a linear orientation, than a bending of the instrument at the proximal portion in a certain plane would result in a bending of the instrument at the distal portion in the same plane but in a 180 degrees opposite directions. This spiral construction of the steering wires 16 allows for the effect that bending of the instrument at the proximal portion in a certain plane may result in a bending of the instrument at the distal portion in another plane, or in the same plane in the same direction. A preferred spiral construction may be such that the end portion of a respective steering element 16 at the proximal portion of the instrument is arranged at an angularly shifted orientation of 180 degrees about the longitudinal axis relative to the end portion of the same steering wire 16 at the distal portion of the instrument. However, e.g. any other angularly shifted orientation, e.g. 90 degrees, is within the scope of this document. The slots 70 are dimensioned such that movement of a steering wire is guided by adjacent steering wires when provided in place in a steerable instrument. However, especially at the flexible zones 13, 14 of the instrument, the width of steering wires 16 may be less to provide the instrument with the required flexibility/bendability at those locations.

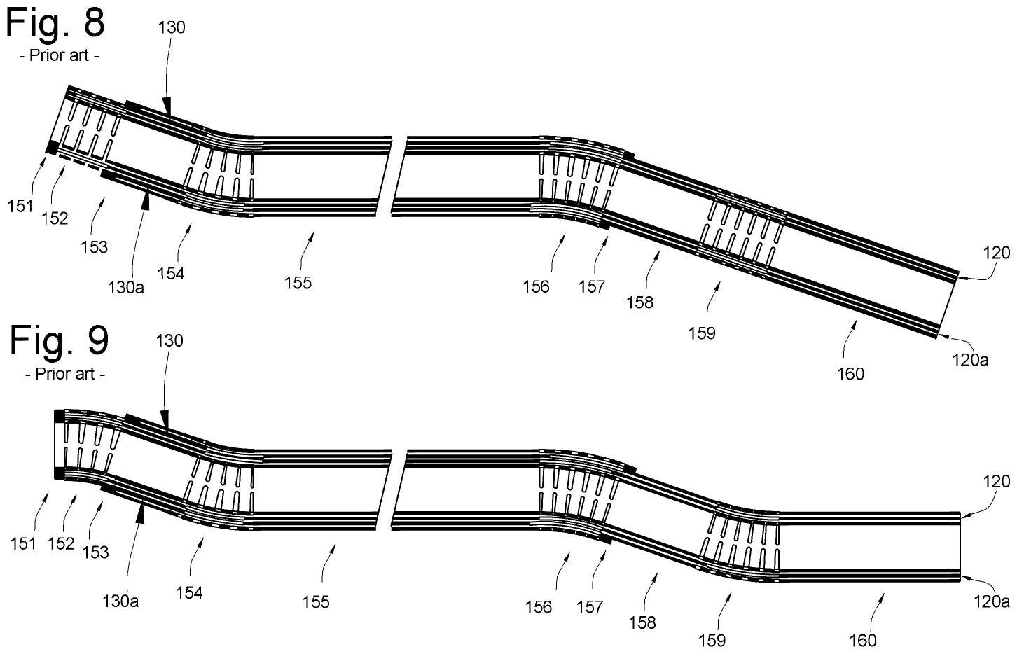

FIG. 5 provides a detailed perspective view of the distal portion of an embodiment of an elongated tubular body 76 of a steerable instrument which has two steerable distal bendable zones 74, 75 which are operated by two bendable proximal zones 72, 73, respectively. FIG. 5 shows that the elongated tubular body 76 comprises a number of co-axially arranged layers or cylindrical elements including an outer cylindrical element 104 that ends after a first distal flexible zone 74 at the distal end portion 13. The distal end portion 13 of the outer cylindrical element 104 is fixedly attached to a cylindrical element 103 located inside of and adjacent to the outer cylindrical element 104, e.g. by means of spot welding at welding spots 100. However, any other suitable attachment method can be used, including any mechanical snap fit connection or gluing by a suitable glue.

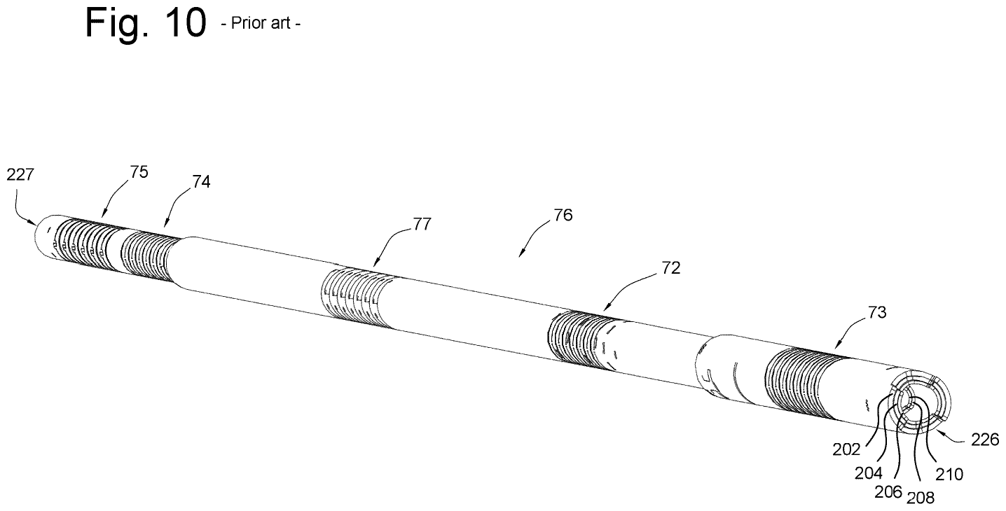

FIG. 6 provides a more detailed view of the distal end part 13 and shows that, in this embodiment, it includes three co-axially arranged layers or cylindrical elements, i.e., an inner cylindrical element 101, a first intermediate cylindrical element 102 and a second intermediate cylindrical element 103. The distal ends of inner cylindrical element 101, first intermediate cylindrical element 102 and second intermediate cylindrical element 103 are all three fixedly attached to one another. This may be done by means of spot welding at welding spots 100. However, any other suitable attachment method can be used, including any mechanical snap fit connection or gluing by a suitable glue. The points of attachment may be at the end edges of inner cylindrical element 101, first intermediate cylindrical element 102 and second intermediate cylindrical element 103, as shown in the figures. However, these points of attachment may also be located some distance away from these edges, be it, preferably, between the end edges and the locations of the flexible zone 75.

It will be clear to the skilled person that the elongated tubular body 76 as shown in FIG. 5 comprises four cylindrical elements in total. The elongated tubular body 76 according to the embodiment shown in FIG. 5 comprises two intermediate cylindrical elements 102 and 103 in which the steering members of the steering arrangement are arranged. However, extra or less cylindrical elements may be provided if desired.

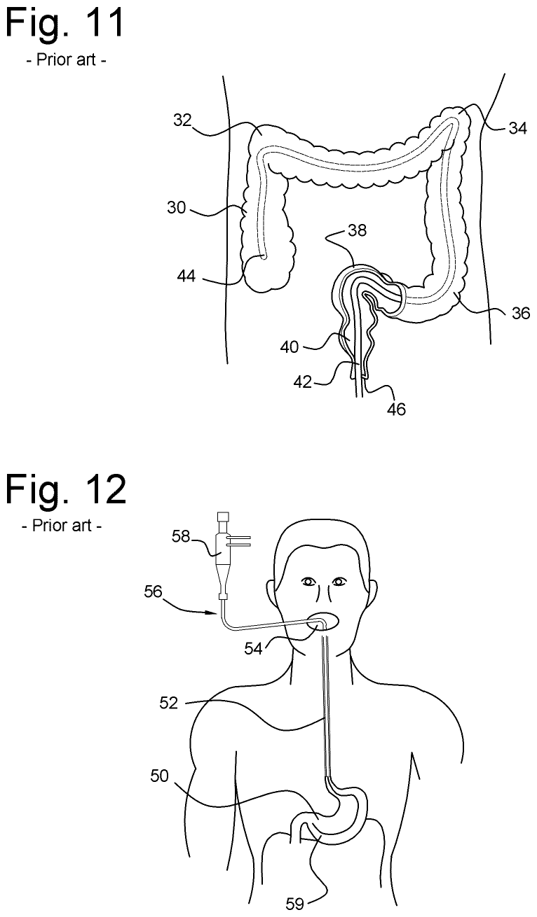

The steering arrangement in the exemplary embodiment of the elongated tubular body 76 as shown in FIG. 5 comprises the two flexible zones 72, 73 at the proximal end part 11 of the elongated tubular body 76, the two flexible zones 74, 75 at the distal end part 13 of the elongated tubular body 76 and the steering members that are arranged between related flexible zones at the proximal 11 and distal 13 end parts. An exemplary actual arrangement of the steering members is shown in FIG. 7, which provides a schematic longitudinal cross-sectional view of the exemplary embodiment of the elongated tubular body 76 as shown in FIG. 5.

Flexible zones 72, 73, 74, and 75 are, in this embodiment, implemented by providing the respective cylindrical elements with slits 72a, 73a, 74a, and 75a, respectively. Such slits 72a, 73a, 74a, and 75a may be arranged in any suitable pattern such that the flexible zones 72, 73, 74, and 75 have a desired flexibility in the longitudinal and tangential direction in accordance with a desired design.

FIG. 7 shows a longitudinal cross section of the four layers or cylindrical elements mentioned above, i.e. the inner cylindrical element 101, the first intermediate cylindrical element 102, the second intermediate cylindrical element 103, and the outer cylindrical element 104.

The inner cylindrical element 101, as seen along its length from the distal end to the proximal end of the instrument, comprises a rigid ring 111, which is arranged at the distal end part 13 of the steerable instrument 10, a first flexible portion 112, a first intermediate rigid portion 113, a second flexible portion 114, a second intermediate rigid portion 115, a third flexible portion 116, a third intermediate rigid portion 117, a fourth flexible portion 118, and a rigid end portion 119, which is arranged at the proximal end portion 11 of the steerable instrument.

The first intermediate cylindrical element 102, as seen along its length from the distal end to the proximal end of the instrument, comprises a rigid ring 121, a first flexible portion 122, a first intermediate rigid portion 123, a second flexible portion 124, a second intermediate rigid portion 125, a third flexible portion 126, a third intermediate rigid portion 127, a fourth flexible portion 128, and a rigid end portion 129. The portions 122, 123, 124, 125, 126, 127 and 128 together form a steering wire 120 that can be moved in the longitudinal direction like a wire. The longitudinal dimensions of the rigid ring 121, the first flexible portion 122, the first intermediate rigid portion 123, the second flexible portion 124, the second intermediate rigid portion 125, the third flexible portion 126, the third intermediate rigid portion 127, the fourth flexible portion 128, and the rigid end portion 129 of the first intermediate element 102, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the inner cylindrical element 101, respectively, and are coinciding with these portions as well. In this description “approximately equal” means that respective same dimensions are equal within a margin of less than 10%, preferably less than 5%.

Similarly, the first intermediate cylindrical element 102 comprises one or more other steering wires of which one is shown with reference number 120a.

The second intermediate cylindrical element 103, as seen along its length from the distal end to the proximal end of the instrument, comprises a first rigid ring 131, a first flexible portion 132, a second rigid ring 133, a second flexible portion 134, a first intermediate rigid portion 135, a first intermediate flexible portion 136, a second intermediate rigid portion 137, a second intermediate flexible portion 138, and a rigid end portion 139. The portions 133, 134, 135 and 136 together form a steering wire 130 that can be moved in the longitudinal direction like a wire. The longitudinal dimensions of the first rigid ring 131, the first flexible portion 132 together with the second rigid ring 133 and the second flexible portion 134, the first intermediate rigid portion 135, the first intermediate flexible portion 136, the second intermediate rigid portion 137, the second intermediate flexible portion 138, and the rigid end portion 139 of the second intermediate cylinder 103, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the first intermediate element 102, respectively, and are coinciding with these portions as well.

Similarly, the second intermediate cylindrical element 103 comprises one or more other steering wires of which one is shown with reference number 130a.

The outer cylindrical element 104, as seen along its length from the distal end to the proximal end of the instrument, comprises a first rigid ring 141, a first flexible portion 142, a first intermediate rigid portion 143, a second flexible portion 144, and a second rigid ring 145. The longitudinal dimensions of the first flexible portion 142, the first intermediate rigid portion 143 and the second flexible portion 144 of the outer cylindrical element 104, respectively, are aligned with, and preferably approximately equal to the longitudinal dimension of the second flexible portion 134, the first intermediate rigid portion 135 and the first intermediate flexible portion 136 of the second intermediate element 103, respectively, and are coinciding with these portions as well. The rigid ring 141 has approximately the same length as the rigid ring 133 and is fixedly attached thereto, e.g. by spot welding or gluing. Preferably, the rigid ring 145 overlaps with the second intermediate rigid portion 137 only over a length that is required to make an adequate fixed attachment between the rigid ring 145 and the second intermediate rigid portion 137, respectively, e.g. by spot welding or gluing. The rigid rings 111, 121 and 131 are attached to each other, e.g., by spot welding or gluing. This may be done at the end edges thereof but also at a distance of these end edges.

In an embodiment, the same may apply to the rigid end portions 119, 129 and 139, which can be attached to one another as well in a comparable manner. However, the construction may be such that the diameter of the cylindrical elements at the proximal portion is larger, or smaller, with respect to the diameter at the distal portion. In such embodiment the construction at the proximal portion differs from the one shown in FIG. 7. As a result of the increase or decrease in diameter an amplification or attenuation is achieved, i.e., the bending angle of a flexible zone at the distal portion will be larger or smaller than the bending angle of a corresponding flexible portion at the proximal portion.

The inner and outer diameters of the cylindrical elements 101, 102, 103, and 104 are chosen in such a way at a same location along the elongated tubular body 76 that the outer diameter of inner cylindrical element 101 is slightly less than the inner diameter of the first intermediate cylindrical element 102, the outer diameter of the first intermediate cylindrical element 102 is slightly less than the inner diameter of the second intermediate cylindrical element 103 and the outer diameter of the second intermediate cylindrical element 103 is slightly less than the inner diameter of the outer cylindrical element 104, in such a way that a sliding movement of the adjacent cylindrical elements with respect to each other is possible. The dimensioning should be such that a sliding fit is provided between adjacent elements. A clearance between adjacent elements may generally be in the order of 0.02 to 0.1 mm, but depends on the specific application and material used. The clearance may be smaller than a wall thickness of the steering wires to prevent an overlapping configuration thereof. Restricting the clearance to about 30% to 40% of the wall thickness of the steering wires is generally sufficient.

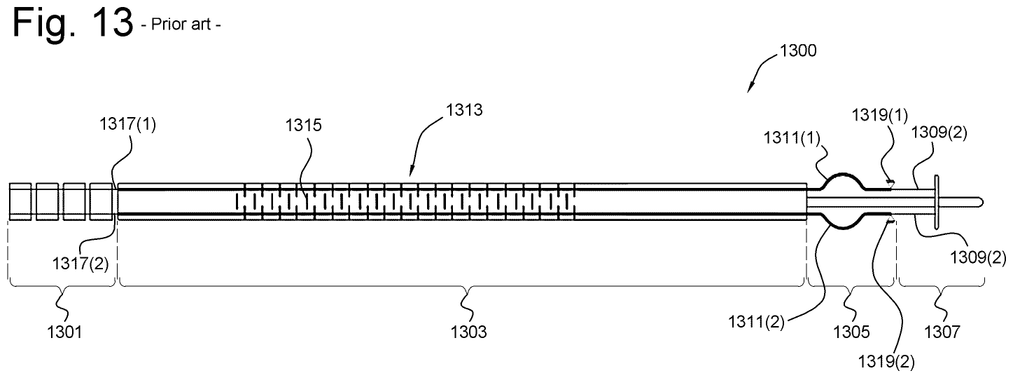

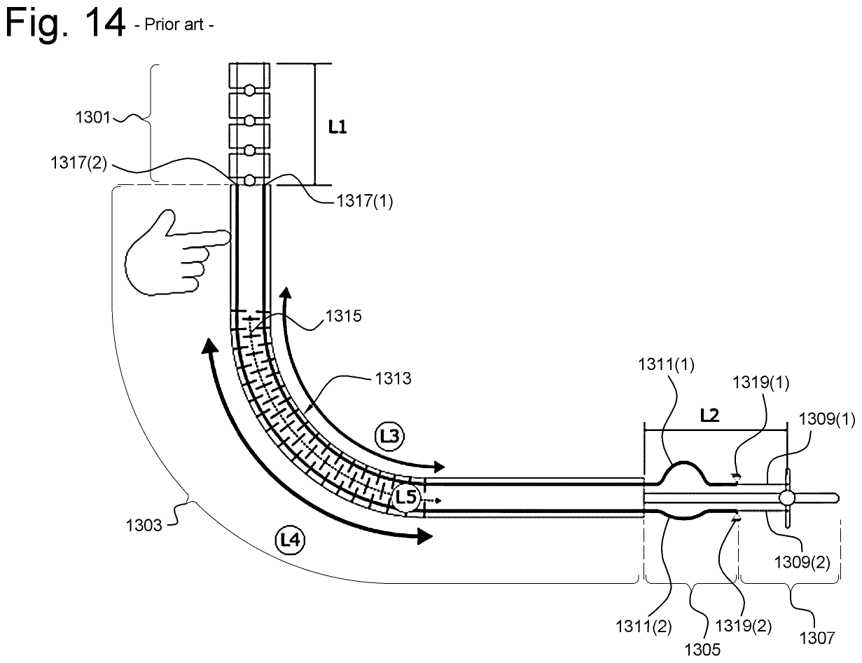

As can be seen in FIG. 7, flexible zone 72 of the proximal end part 11 is connected to the flexible zone 74 of the distal end part 13 by portions 134, 135 and 136, of the second intermediate cylindrical element 103, which form a first set of steering wires of the steering arrangement of the steerable instrument. Furthermore, flexible zone 73 of the proximal end part 11 is connected to the flexible zone 75 of the distal end part 13 by portions 122, 123, 124, 125, 126, 127, and 128 of the first intermediate cylindrical element 102, which form a second set of steering wires of the steering arrangement. The use of the construction as described above allows the steerable instrument 10 to be used for double bending. The working principle of this construction will be explained with respect to the examples shown in FIGS. 8 and 9.

For the sake of convenience, as shown in FIGS. 7, 8 and 9, the different portions of the cylindrical elements 101, 102, 103, and 104 have been grouped into zones 151-160 that are defined as follows. Zone 151 comprises the rigid rings 111, 121, and 131. Zone 152 comprises the portions 112, 122, and 132. Zone 153 comprises the rigid rings 133 and 141 and the portions 113 and 123. Zone 154 comprises the portions 114, 124, 134 and 142. Zone 155 comprises the portions 115, 125, 135 and 143. Zone 156 comprises the portions 116, 126, 136 and 144. Zone 157 comprises the rigid ring 145 and the parts of the portions 117, 127, and 137 coinciding therewith. Zone 158 comprises the parts of the portions 117, 127, and 137 outside zone 157. Zone 159 comprises the portions 118, 128 and 138. Finally, zone 160 comprises the rigid end portions 119, 129 and 139.

In order to deflect at least a part of the distal end part 13 of the steerable instrument, it is possible to apply a bending force, in any radial direction, to zone 158. According to the examples shown in FIGS. 8 and 9, zone 158 is bent downwards with respect to zone 155. Consequently, zone 156 is bent downwards. Because of the first set of steering wires comprising portions 134, 135, and 136 of the second intermediate cylindrical element 103 that are arranged between the second intermediate rigid portion 137 and the second rigid ring 133, the downward bending of zone 156 is transferred by a longitudinal displacement of the first set of steering wires into an upward bending of zone 154 with respect to zone 155. This is shown in both FIGS. 8 and 9.

It is to be noted that the exemplary downward bending of zone 156, only results in the upward bending of zone 154 at the distal end of the instrument as shown in FIG. 8. Bending of zone 152 as a result of the bending of zone 156 is prevented by zone 153 that is arranged between zones 152 and 154. When subsequently a bending force, in any radial direction, is applied to the zone 160, zone 159 is also bent. As shown in FIG. 9, zone 160 is bent in an upward direction with respect to its position shown in FIG. 8. Consequently, zone 159 is bent in an upward direction. Because of the second set of steering wires comprising portions 122, 123, 124, 125, 126, 127 and 128 of the first intermediate cylindrical element 102 that are arranged between the rigid ring 121 and the rigid end portion 129, the upward bending of zone 159 is transferred by a longitudinal displacement of the second set of steering wires into a downward bending of zone 152 with respect to its position shown in FIG. 8.

FIG. 9 further shows that the initial bending of the instrument in zone 154 as shown in FIG. 8 will be maintained because this bending is only governed by the bending of zone 156, whereas the bending of zone 152 is only governed by the bending of zone 159 as described above. Due to the fact that zones 152 and 154 are bendable independently with respect to each other, it is possible to give the distal end part 13 of the steerable instrument a position and longitudinal axis direction that are independent from each other. In particular the distal end part 13 can assume an advantageous S-like shape. The skilled person will appreciate that the capability to independently bend zones 152 and 154 with respect to each other, significantly enhances the manoeuvrability of the distal end part 13 and therefore of the steerable instrument as a whole.

Obviously, it is possible to vary the lengths of the flexible portions shown in FIGS. 7 to 9 as to accommodate specific requirements with regard to bending radii and total lengths of the distal end part 13 and the proximal end part 11 of the steerable instrument or to accommodate amplification or attenuation ratios between bending of at least a part of the proximal end part 11 and at least a part of the distal end part 13.

In the shown embodiment, the steering wires comprise one or more sets of steering wires that form integral parts of the one or more intermediate cylindrical elements 102, 103. Preferably, the steering wires comprise remaining parts of the wall of an intermediate cylindrical element 102, 103 after the wall of the intermediate cylindrical element 102, 103 has been provided with longitudinal slits that define the remaining steering wires.

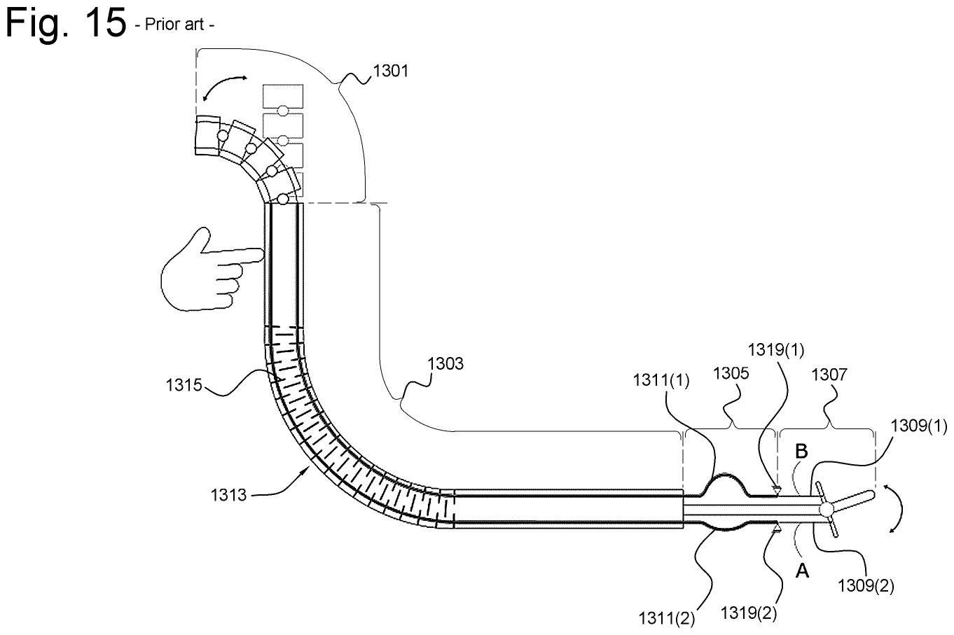

FIG. 10 shows a 3D view of an example of a steerable instrument. Like reference numbers refer to the same elements as in other figures. Their explanation is not repeated here. The instruments comprises five coaxial cylindrical elements 202-210. An inner cylindrical element 210 is surrounded by intermediate cylindrical element 208 which is surrounded by intermediate cylindrical element 206 which is surrounded by intermediate cylindrical element 204 which is, finally surrounded by outer cylindrical element 202. Inner intermediate cylindrical element may be made of a flexible spiraling spring. The proximal and distal ends, respectively, of the instrument are indicated with reference numbers 226 and 227, respectively.

As shown, here, instrument 76 comprises a flexible zone 77 in its intermediate part between flexible zone 72 and flexible zone 74. I.e., intermediate cylindrical element 204 (which is located at the outer side in the area of flexible zone 77) is provided with a slotted structure to provide intermediate cylindrical element with a desired flexibility. The longitudinal length of the slotted structure in flexible zone 77 depends on the desired application. It may be as long as the entire part between flexible zones 72 and 74. All other cylindrical elements 206, 208, 210 inside intermediate cylindrical element 204 are also flexible in flexible zone 77. Those cylindrical elements that have steering wires in the flexible zone 77 are flexible by way of definition. Others are provided with suitable hinges, preferably made by suitable slotted structures.

Some locations to be operated in a body need specifically designed instruments. E.g., by making the intermediate part 12 of the instrument completely flexible, the instrument can also be used in areas in the body which are only accessible via curved natural access guides/channels, like the colon, the stomach via the oesophagus or the heart via curved blood vessels.

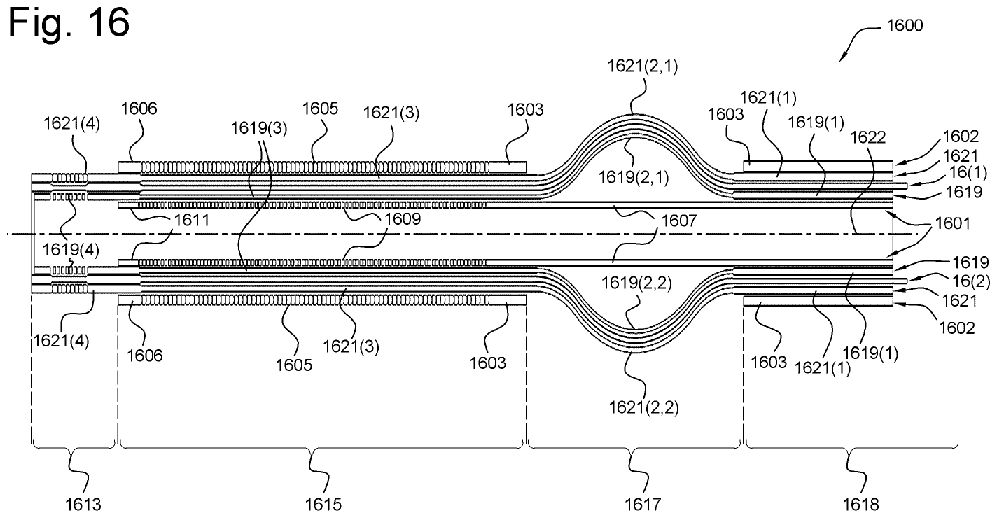

The instrument can e.g. be designed to be used as a colonoscope. FIG. 11 shows a schematic view of a colonoscope 42 in use. The colonoscope 42 is inserted into a colon 30 of a human body. Typically, the colon 30 has several almost square angled sections 32, 34, 36, and 38. If a surgeon needs to operate an area of the colon 30 upstream from square angled section 32 the colonoscope 42 needs to be inserted into the colon 30 along a distance of up to 1.5 meter. Moreover, the colonoscope 42 needs to be so flexible that it can be guided from an anus through all squared angled sections 32-38 of the colon 30 easily without risks of damaging the inner wall of the colon 30.

In operation, usually, several invasive instruments are inserted through the colonoscope 42 to provide one or more tools for some function at its distal end 44. In colonoscopy, such a tool typically includes a camera lens and a lighting element. To assist the surgeon in steering the camera view to the desired location and view in colon 30, typically, the distal end is deflectable from a longitudinal axis in all angular directions. This also holds for the inserted instruments with tools 2. That can be implemented by providing such an instruments with one or more deflectable zones, like the deflectable zones 16, 17 of the instrument shown in FIGS. 5-10. These distal deflectable zones are controlled by suitable steering cables accommodated in the instruments connected to a suitable steering mechanism at the proximal ends of the instruments.

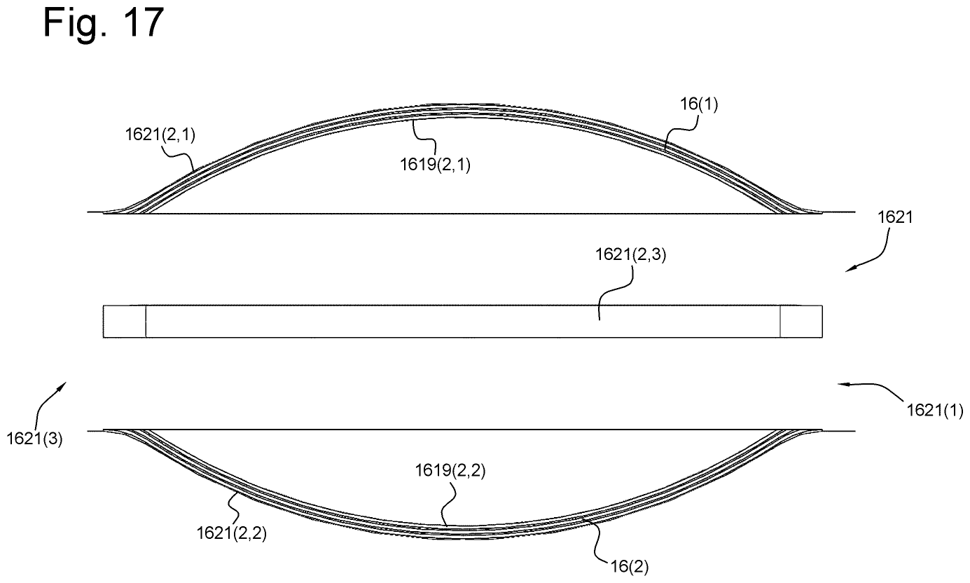

FIG. 12 shows a schematic view of a gastroscope 56 in use. The gastroscope 56 is inserted into a stomach 50 of a human body via mouth, oral cavity/throat 54 and oesophagus 52. Especially when a surgeon needs to operate a lower portion of the stomach 50, the gastronoscope 56 needs to be guided through several curved/angled sections. Therefore, the gastroscope 56 needs to be flexible such that there is little risk of damaging inner walls of the mouth/throat 54, oesophagus 52 and stomach 50.

In operation, usually, several invasive instruments are inserted through the gastroscope 56 to provide one or more tools for some function at its distal end 59. In gastroscopy, such a tool typically includes a camera lens and a lighting element. To assist the surgeon in steering the camera view to the desired location and direction in stomach 50, typically, the distal end 59 of the gastrocope 56 is deflectable from a longitudinal axis in all angular directions. This also holds for the inserted instruments with tools 2. That can be implemented by providing such an instrument with one or more deflectable zones, like the deflectable zones 16, 17 of the instrument shown in FIGS. 5-10. These distal deflectable zones are controlled by suitable steering cables accommodated in the instruments connected to a suitable steering mechanism of these instruments.

Instruments according to the invention can be used in such colonoscopes and gastroscopes but also in other applications like instruments designed for entering the lung bronchi. Requirements to such an instrument may be that they show a high rotational stiffness, high longitudinal stiffness, bending flexibility along its entire length and accurate and repeatable deflectability of a steerable tip even in cases of long instruments, e.g., longer than 1 m, and with a relatively small diameter that fits to the working channels within or attached to colonoscopes and gastroscopes.

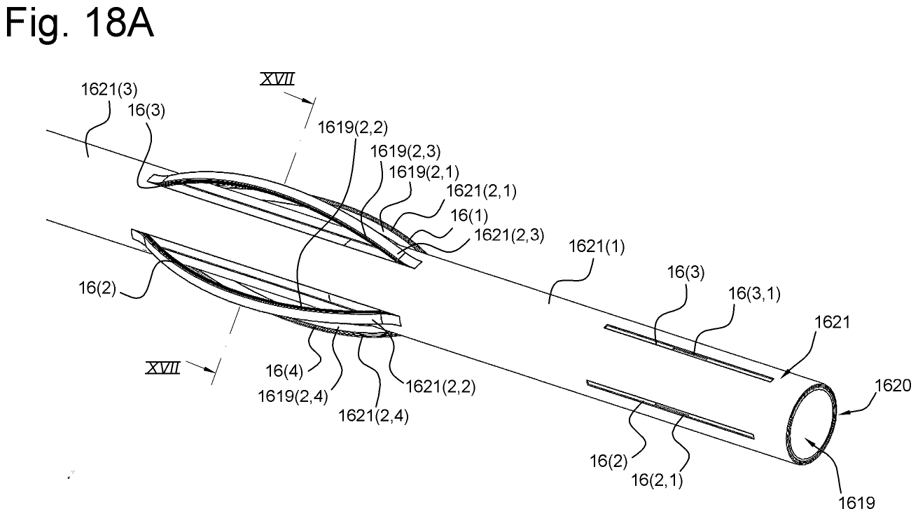

In order to better understand the issues dealt with in the present document, first a detailed description is provided of a prior art flexible steerable instrument with Bowden cables with reference to FIGS. 13-15.

A Bowden cable as known from the prior art can be defined as a type of flexible cable used to transmit mechanical force or energy by the movement of an inner cable relative to a hollow outer cable housing. The housing is generally of composite construction, consisting of an inner lining, a longitudinally incompressible layer such as a helical winding or a sheath of steel wire, and a protective outer covering. The cable housing is often called a coil pipe. Here, the term steering wire guiding will be used for the outer cable housing.

FIG. 13 shows an instrument according to prior art with a steerable tip and a flexible body, with Bowden cable arrangements in a length compensation section at one end of the Bowden cable. Each Bowden cable arrangement comprises a steering wire and a surrounding steering wire guiding. The steering wire guiding is attached to the instrument body in the distal end before the proximal end of the steerable tip and is also attached to the instrument body in the proximal end. The length compensation section is positioned between the distal end of the guiding and the proximal end of the guiding, preferably in the proximal end of the instrument. In this drawing the length compensation section has a simple geometry of a curve that can be shortened or elongated. But prior art provides many different solutions for a length compensation section.

More in detail FIG. 13 shows a flexible steerable instrument 1300 with a tip section 1301, a flexible body section 1303, a length compensation section 1305, and a steering section 1307 which may comprise a handle. The length compensation section 1305 may be arranged at another longitudinal location, e.g., somewhere inside the body section 1303.

Steering wires 1309(1), 1309(2) run from the steering section 1307 to the tip section 1301 in order to allow bending the tip section 1301 relative to body section 1303. Inside body section 1303 and length compensation section 1305, steering wires 1309(1), and 1309(2), respectively, are arranged inside steering wire guidings 1311(1) and 1311(2), respectively. Steering wire guidings 1311(1) and 1311(2), respectively, are held at a fixed position 1317(1) and 1317(2), respectively, at the transition between tip section 1301 and body section 1303. Similarly, steering wire guidings 1311(1) and 1311(2), respectively, are held at a fixed position 1319(1) and 1319(2), respectively, at the transition between length compensation section 1305 and steering section 1307.

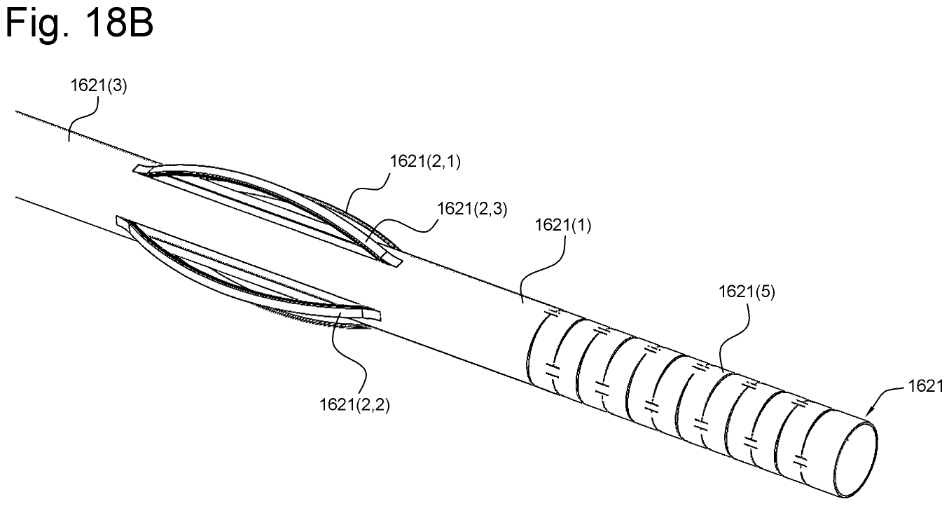

FIG. 14 shows the same instrument as FIG. 13, but now body section 1303 is bent. Tip section 1301 is not bent. The portions of steering wires 1309(1) and 1309(2) inside tip section 1301 have a length L1. The inside curve of the curved part, i.e., the part of hinge 1313, of the body section 1303 has a length L3, and the outside curve has a length L4. The initial length of steering wire guidings 1311(1), 1311(2) inside body section 1303, i.e. FIG. 13, is L5. The total length of steering wires 1309(1), 1309(2) inside length compensation section 1305 inside the steering section 1307 is L2.

Due to the bending, L3 is shorter than the initial steering wire guiding length L5 and L4 is longer than the initial steering wire guiding length L5. If one wants that L1 and L2 after the bending are equal to L1 and L2 before the bending, in other words, if one does not want that the tip or the steering device in steering section 1307 deflects due to bending of the body, the length difference between L5 and L3 or L4 has to be absorbed by the length compensation section 1305 as is shown in the drawing. In this drawing, the length compensation section 1305 absorbs the length differences by increasing the curve height of steering wire guiding 1311(1) with the associated portion of steering wire 1309(1) inside (which can absorb a longer length of steering wire guiding 1311(1) with steering wire 1309(1) outside body section 1303) and decreasing the curve height of steering wire guiding 1311(2) with the associated portion of steering wire 1309(2) inside (which can absorb a shorter length of steering wire guiding 1311(2) with steering wire 1309(2) outside body section 1303). In this way, tip defection is fully isolated from body deflection.

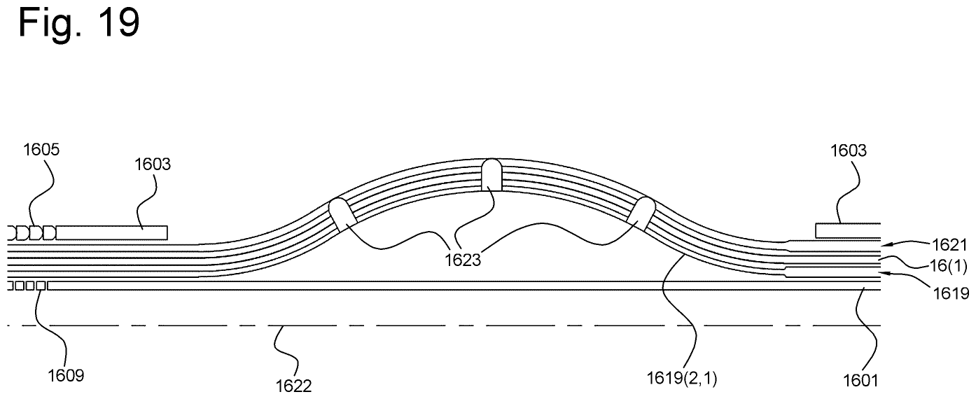

FIG. 15 shows the same instrument as in FIGS. 13 and 14, but now the steering device is deflected for steering the instrument tip section 1301. When the tip section 1301 is deflected as shown, a pull force is generated in steering wire 1309(2) and a push force is generated in steering wire 1309(1). This results in compression or stretching forces in the corresponding body section 1303 and tip section 1301 areas. The steering wire guidings 1311(1) and 1311(2) are flexible enough to allow body section bending but are very stiff in their own longitudinal direction so that they can withstand the compression or stretching forces without significant deformation. The length compensation section 1305 itself has a longitudinal stiffness designed such that it can withstand the compression or elongation forces due to pulling or pushing the steering wires 1309(1), 1309(2) without significant deformation. So, in this configuration the compression and elongation forces are fully absorbed by the Bowden cable arrangement inside length compensation section 1305. Therefor the body section 1303 will not compress or elongate anymore and therefor will not bend. In this way, body section steering as an unwanted result of tip steering is prevented.

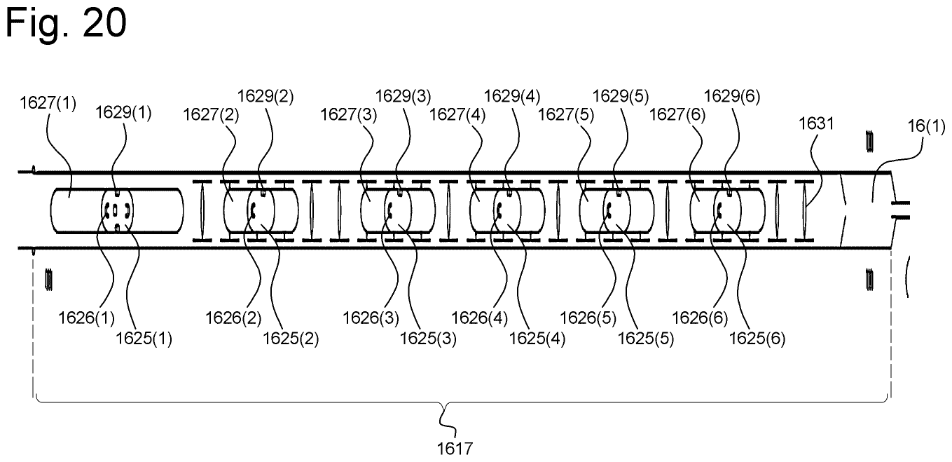

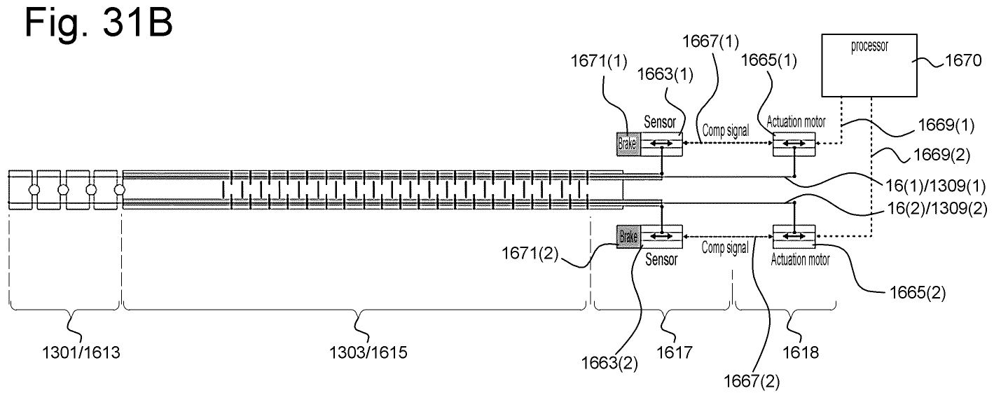

FIGS. 16-31c are embodiments of the invention. FIGS. 16-30 relate to flexible invasive steerable instruments in which steering wires are implemented by steering wires 16(i) (i=1, 2, . . . , I) made from portions of one or more tubes, e.g., by means of (laser) cutting a suitable slot pattern in such one or more tubes, as explained in detail with reference to FIGS. 1-10. As will be explained in detail, steering wire guidings are implemented by steering wire guiding portions also made from one or more tubes. The embodiments of FIGS. 31a-31c can also be implemented with classic steering wires and coil pipes.

FIG. 16 shows a longitudinal cross section through a flexible steerable invasive instrument with a tip section 1613, a flexible body section 1615, a length compensation section 1617, and a steering section 1618 which may comprise a handle, a deflectable steering unit or a robotic steering unit. The length compensation section 1617 may be arranged at another longitudinal location, e.g., somewhere inside the body section 1615 or be integrated inside the steering section 1618. The right hand side is the proximal end and the left hand side is the distal end of the instrument.

The instrument as shown is made of five tubes coaxially arranged about a central axis 1622. However, other numbers can be applied as well. An inner tube 1601 is arranged inside the instrument, which is flexible at least inside the body section 1615 but stiff in its longitudinal direction. To that end the inner tube 1601 may be provided with suitable hinges 1609 in the body section 1615, made by providing the inner tube 1601 with a suitable pattern of slots. The portions of inner tube 1601 at the transition between tip section 1613 and body section 1615 is indicated with reference sign 1611, and inside length compensation section 1617 with reference sign 1607.

A first intermediate tube 1619 surrounds inner tube 1601. First intermediate tube 1619 has a steering section portion 1619(1) which may be entirely ring shaped, a plurality of inside steering wire guiding portions 1619(2,i), i.e. one for each steering wire 16(i), in length compensation section 1617, a body section portion 1619(3) which may be ring shaped, and a tip section portion 1619(4) which may be ring shaped and provided with one or more suitable hinges. First intermediate tube 1619 and inner tube 1601 are, preferably, attached to one another at the transition between tip section 1613 and body section 1615 and at the proximal side between the length compensation section 1617 and the steering section 1618, e.g. by laser welding, gluing, etc. Body section portion 1619(3) is flexible at least in the flexible portions of body section 1615.

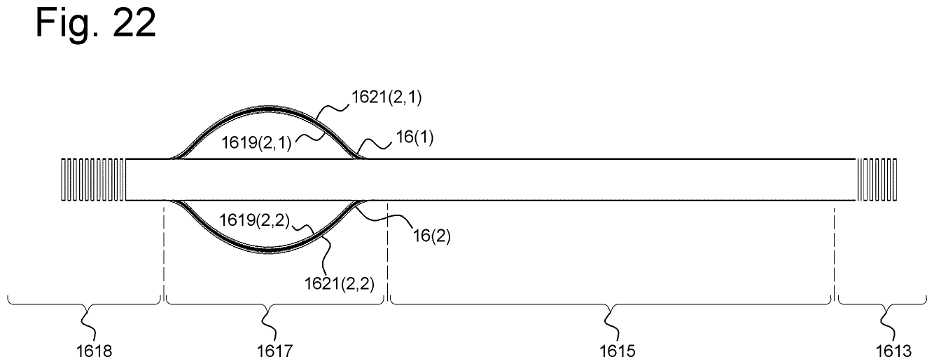

A second intermediate tube 1620 (cf. e.g. FIG. 18a) surrounds first intermediate tube 1619. Second intermediate tube 1620 has steering wires 16(i) running from the proximal end to the distal end of the instrument. These steering wires should be flexible in at least the flexible part of tip section 1613, the flexible part of body portion 1615, and inside length compensation section 1617.

A third intermediate tube 1621 surrounds second intermediate tube 1620. Third intermediate tube 1621 has a steering section portion 1621 (1) which may be entirely ring shaped, a plurality of outside steering wire guiding portions 1621(2,i), i.e. one for each steering wire 16(i), a length compensation section 1617, a body section portion 1621(3) which may be ring shaped, and a tip section portion 1621(4) which may be ring shaped and provided with one or more suitable hinges. Body section portion 1621(3) is flexible at least in the flexible portions of body section 1615.

At the distal end of the instrument, i.e., distally beyond the flexing portion of tip section 1613, all steering wires 16(i) are attached to at least one of first and third intermediate tubes 1619 and 1621 to allow for pulling and pushing forces to be transferred to the tip section 1613.

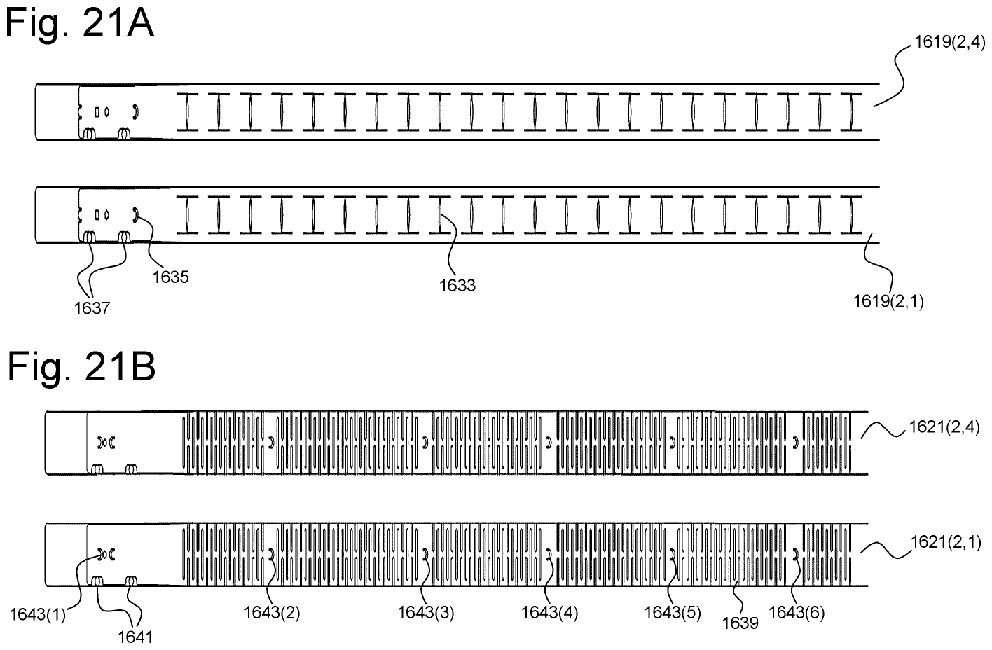

An outer tube 1602 surrounds third intermediate tube 1621. Outer tube 1602, in the shown example, has—as seen from the proximal end to the distal end—a steering section portion 1604 which may be entirely ring shaped, one or more open portions in length compensation section 1617 in order to allow the length compensation portions of tubes 1619, 1620 and 1621 to increase their extension away from central axis 1622 if desired, a ring shaped portion 1603, a flexible portion 1605 and a ring shaped portion 1606. Ring shaped portion 1606 may be attached to third intermediate tube 1621 at the transition between tip section 1613 and body section 1615. FIG. 17 shows an enlarged outside view of length compensation section 1617. In

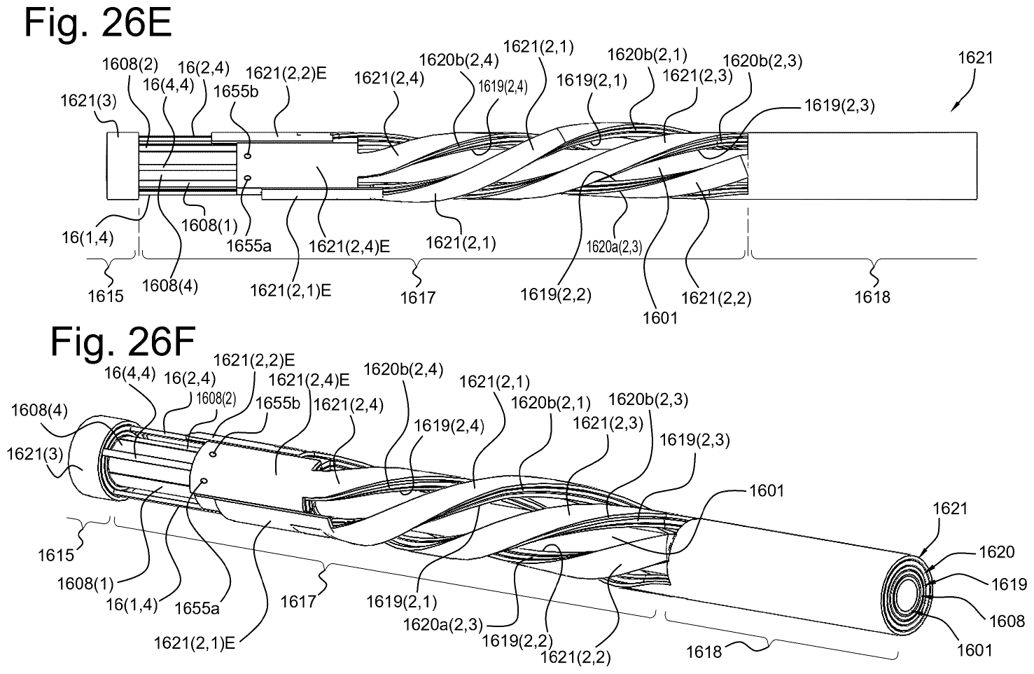

this figure, one now also sees an outside steering wire guiding portion 1621(2,3) located on top of a steering wire 16(3) (not visible in this figure). The steering wire guiding portions 1619(2,i), 1621(2,i) are bent radially outward, with the steering element 16(i) in between. The steering element 16(i) is now radially guided by the steering wire guiding portions 1619(2,i), 1621(2,i). Note that FIGS. 25A, 25B show more details of how steering wire guiding portions 1619(2,i), 1621(2,i) may be constructed.

FIG. 18a shows an embodiment were the proximal instrument end is configured for coupling it to for example a robotic steering device or a separate handheld steering device. To that end, each steering wire 16(i) may be provided with an opening 16(i,1) which is arranged in steering section 1618 and radially inside a suitably sized slot in third intermediate tube 1921. Each opening 16(i,1) is configured for receiving an associated mechanical coupling unit of the robotic steering device such that the robotic steering device can individually operate the steering wires 16(i) by means of these mechanical coupling units. Instead of openings 16(i,1) other coupling mechanisms can be used.

FIG. 18b shows the same instrument, but now the proximal end of the instrument is configured for steering the distal tip with a bendable section in a similar way as shown in FIGS. 1-10. Of course other steering means like a gimballing handle or other known steering methods can be envisioned.

The steering wires cross section may have a substantially rectangular shape such that they bend easily in the radial direction, but they are difficult to be bent in the tangential direction. In that case, it is not necessary to also guide the steering wires 16(i) in tangential direction in the length compensation section 1617. I.e., they will stay between the inner and outer steering wire guiding portions 1619(2,i) and 1621(2,i) inside the length compensation section 1617 whilst pulled or pushed.

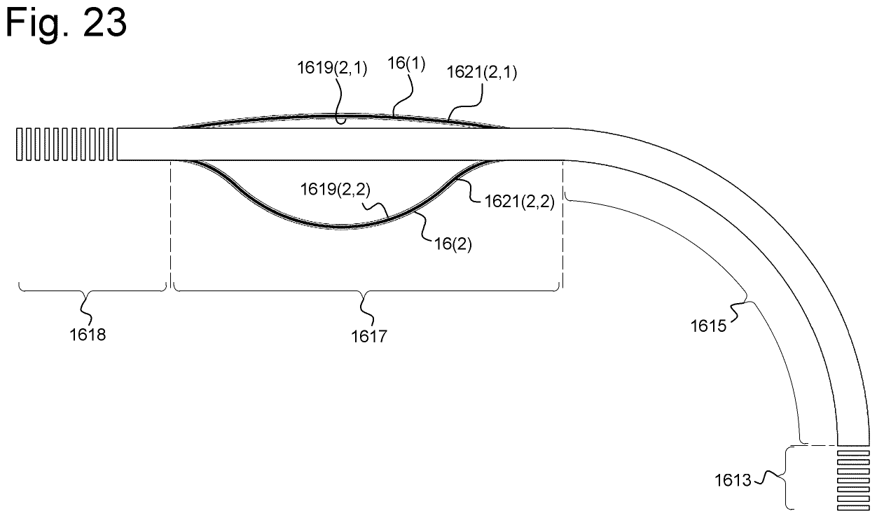

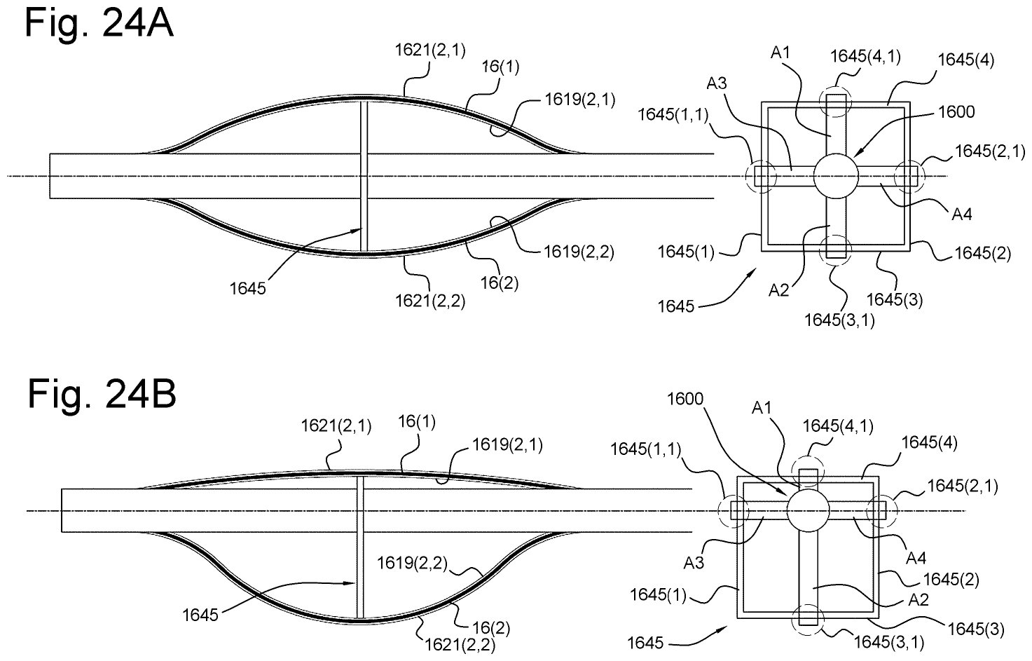

In case one uses steering elements 16(i) that are also flexible in tangential direction, it might be necessary to also guide the steering wires 16(i) in the tangential direction of the instrument. This can be accomplished as is shown in FIGS. 19 and 20.

FIG. 19 shows lips 1623 on the tangential side of inner steering wire guiding portion 1619(2,i), that can be bent upward towards the outer steering wire guiding portion 1621(2,i) and that can, for instance, be permanently attached to the outer steering wire guiding portion 1621(2,i), by for example brazing, soldering or welding or a snap fit connection.

FIG. 20 shows another embodiment in which the steering wires are prevented from tangential movement by means of islands located in respective openings in the respective steering wires 16(i), which islands are attached to at least one of steering wire guiding portions 1619(2,1) and 1621(2,1). As shown in FIG. 20, in length compensation section 1617, steering wire 16(1) is provided with a plurality of openings 1627(j) (j=1, 2, 3, . . . , J; six are shown) each having an island 1625(j) inside. Islands 1625(j) are also cut from tube 1620 and are, when the cutting is finished, still connected to adjacent material of steering wire 16(1) by means of one or more fracture elements 1629(j). Then during assembly these islands 1625(j) can be permanently attached to at least one of steering wire guiding portions 1619(2,1) and 1621(2,1) at an attachment portion 1626(j). This attachment may be done by laser welding, gluing, or bending a lip inside opening 1627(j) such that the distance between steering wire guiding portions 1619(2,1) and 1621(2,1) is larger than the thickness of steering wire 16(1), thus making a radial cage and reducing radial friction. In this way, radial spacers are made, as explained in detail in WO2019009710A1. After attachment, with for example laser welding, the fracture element 1629(j) will fracture upon first activation of steering wire 16(1).

Fracture elements 1629(j) should be designed in the following way. Before being fractured, each fracture element 1629(j) is attached to opposite portions of island 1627(j) and steering wire 16(i). These opposite portions of island 1627(j) and steering wire 16(i) have a geometrical shape such that the stresses in the fracture element 1629(j) are higher than in the surrounding material and/or structure. Therefore, if a deflection or a high enough force is applied on a structure with a fracture element 1629(j) the stress in the fracture element 1629(j) rises above the yield stress of the tube material, causing permanent deflection of fracture element 1629(j). Applying even more deflection or a higher force results in the stress reaching the ultimate tensile stress causing a fracture of fracture element 1629(j). An other mechanism to break the fracture element is achieved by applying low or high cycle fatigue to fracture element 1629(j). The stress in fracture element 1629(j) is raised above the fatigue limit, causing a fatigue fracture. In all cases the stresses in the surrounding structure/material, i.e., island 1627(j) and steering wire 16(i), stays at least below the yield stress of the tube material.

The examples of FIGS. 19 and 20 can be implemented with respect to any steering wire 16(i).

In this way, the embodiments of FIGS. 19 and 20 provide for tangential guiding of the steering wires 16(i) and, here, also the inner steering wire guiding portion 1619(2,i) may be attached to the outer steering wire guiding portion 1621(2,i) so that a virtual fully closed steering wire guide is created for each one of them. Of course, also other methods of radially attaching the inner steering wire guiding portions 1619(2,i) and outer steering wire guiding portions 1621(2,i), as well as creating a tangential guide can be envisioned. In the remaining sections of the instrument the steering wires 16(i) may be guided radially and tangentially by the surrounding tubes and portions of tube 1620, including suitable spacers, that lay next to the steering element. Radial spacers as explained in WO2019009710A1 can be applied as well.

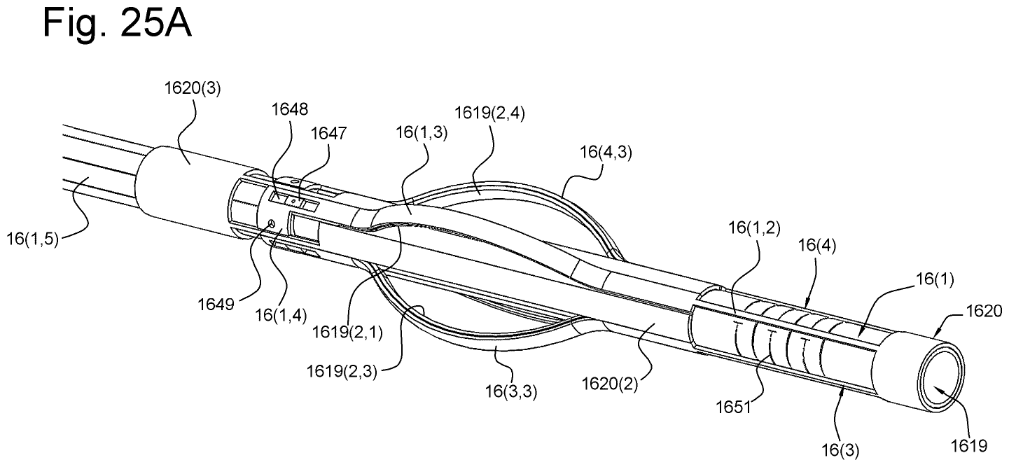

FIGS. 21a and 21b show details of the created steering wire guide in which elastic deformation of the curve shape is made more flexible by cutting slots in steering wire guiding portions 1619(2,i) and 1621(2,i) such that the bending stiffness is less than of a solid strip and such that the length of the outer steering wire guiding portion 1621(2,i) can adapt to the length of the inner steering wire guiding portion 1619(2,i) or visa versa when the curved shape is bent. FIG. 21a shows such slots 1633 in inner steering wire guiding portion 1619(2,1) and 1619(2,4), whereas FIG. 21b shows such slots 1639 in steering wire guiding portion 1621(2,1) and 1621(2,4).

FIG. 21a shows the situation directly after the cutting process is finished and steering wire guiding portion 1619(2,1), 1619(2,4) is still attached to surrounding material of tube 1619 by means of fracture elements 1637. The figure also shows an attachment portion 1635 to which attachment portion 1626(1) of island 1625(1) can be attached. As indicated above, other islands 1625(j) may be attached to inner steering wire guiding portion 1619(2,1) as well.

FIG. 21b shows the situation directly after the cutting process is finished and steering wire guiding portion 1621(2,1), 1621(2,4) is still attached to surrounding material of tube 1621 by means of fracture elements 1641. The figure also shows attachment portions 1643(j) to which attachment portions 1626(j) of islands 1625(j) can be attached.

The result is a longitudinally more flexible length compensation section 1617 that requires less forces to compress or elongate. This on its turn reflects in an easier bendable body section 1615 of the instrument. Another obtainable result is that when the curve shape in length compensation section 1617 is bent outward or inward, one can balance the longitudinal flexibility of the inner and outer steering wire guiding portions 1619(2,i), 1621(2,i) such, that the average length of the inner and outer steering wire guiding portions 1619(2,i), 1621(2,i) stays exactly equal to the length of the enclosed steering wire 16(i). The bending flexibility should be designed such that the longitudinal flexibility of the length compensation section 1617 still can withstand the tip steering forces without significant elongation or shortening.

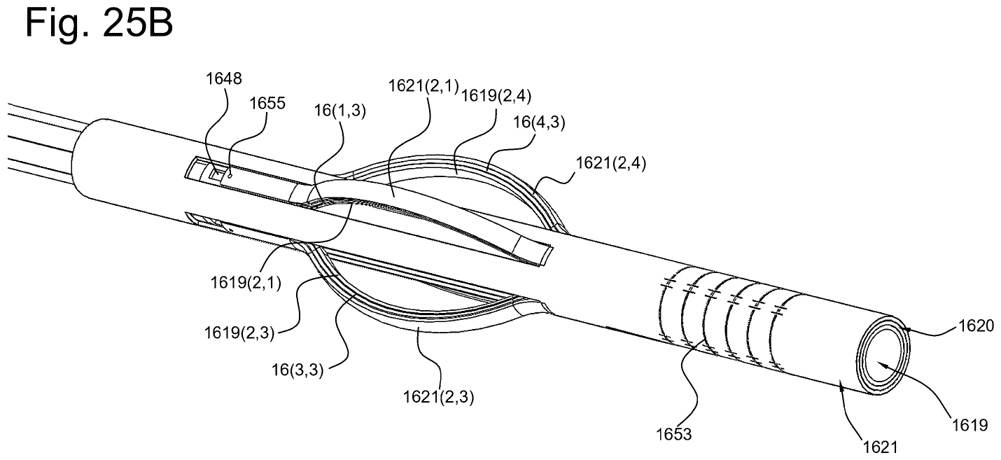

FIG. 22 shows a simplified presentation of a multi tube instrument according to the invention. Each set of inner and outer steering wire guiding portions 1619(2,i), 1621(2,i) with associated portion of steering wire 16(i) forms a length compensation element which is pre-shaped in a certain curve.

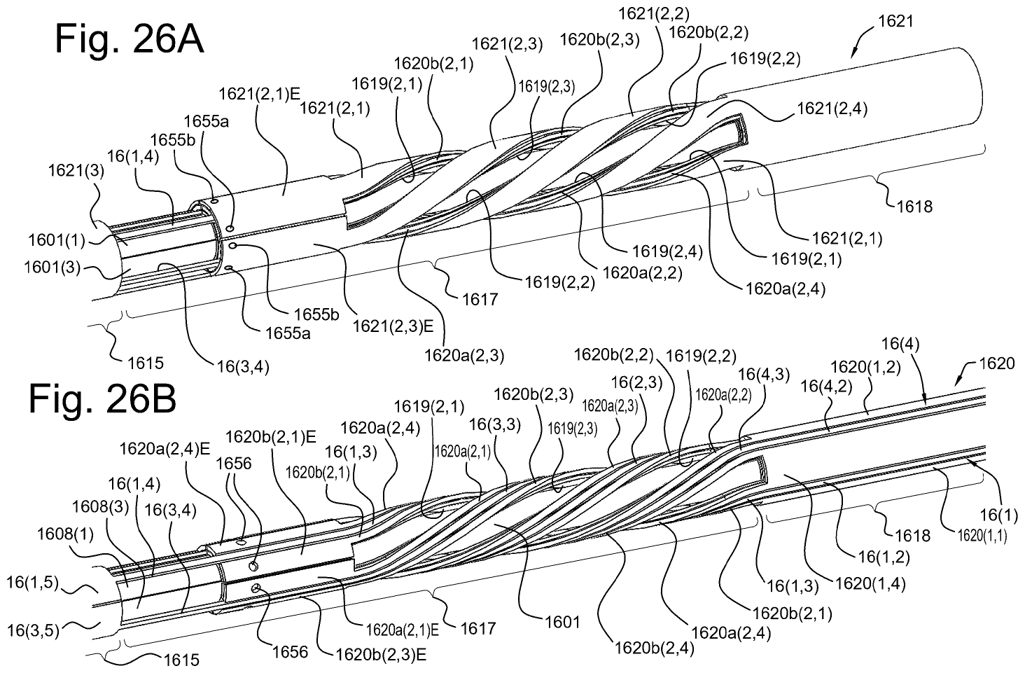

FIG. 23 shows the same multi tube instrument as FIG. 22 and how the length compensation element works as was explained before. When the body section 1615 is bent, the curved shape of the length compensation element will deform such that it absorbs the length differences of the inner and outer steering wire guiding portions 1619(2,i), 1621(2,i) and steering wires 16(i) inside body section 1615 as initiated by bending of body segment 1615. Note that, in this way, bending of body section 1615 has no influence on the deflection angles of tip section 1613 and steering section 1618.

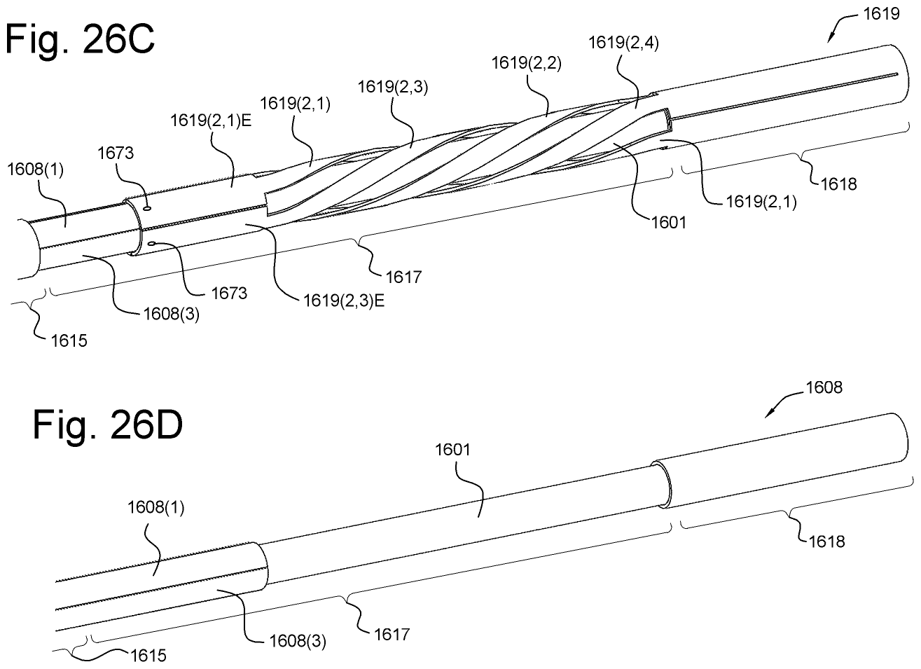

FIGS. 24a and 24b show an embodiment of an instrument according to the invention in which length change of length compensation section 1615 as viewed in a direction perpendicular to central axis 1622 can be prevented, whilst steering the distal tip of the instrument.

FIG. 24a shows the situation in which body section 1615 is not bent and, thus, the neutral position, whereas FIG. 24b shows the situation in which body section 1615 is bent and length compensation section 1617 is active.

In this embodiment, a square frame element 1645 is provided which has a first bar 1645(1) and second bar 1645(2) extending in a first direction and a third bar 1645(3) and fourth bar 1645(4) extending in a second direction perpendicular to the first direction. The right hand side of FIGS. 24a, 24b shows this square frame element 1645 as viewed in the longitudinal direction of the instrument towards the distal end. Reference signs A(i) indicate a set of a steering wire 16(i) with its surrounding steering wire guiding portions 1619(2,i), 1621(2,i). These sets A(i) have a curved shape as shown in the left hand side of FIGS. 24a, 24b. The apex of set A(1), A(2), A(3), A(4), respectively, is slidably connected to bar 1645(4), 1645(3), 1645(1), 1645(2), respectively, with a connection unit 1645(4,1), 1645(3,1), 1645(1,1), 1645(2,1), respectively. Thus, the apex of each set A(i) is restricted in its movement radially but can slide along its associated bar 1645(1), 1645(2), 1645(3), 1645(4) in a direction perpendicular to the radial direction.

It can be shown that the distance between two opposite sets A(1)/A(3), A(2)/A(4) changes slightly when body section 1615 bends. This slight distance change can be compensated for by using bars 1645(1), 1645(2), 1645(3), 1645(4) that are resilient in the radial direction of instrument 1600.

An interesting application is holding the square frame element 1645 fixed in place, e.g. the situation of FIG. 24a or 24b, whilst steering the distal tip by longitudinally moving steering wires 16(i). Then, all sets A(i) are kept in place and will counteract any tendency of the body section 1615 to bend (any further) due to the exerted steering wire forces.

Another advantage of connecting the apexes of the curved length compensation elements is that it can improve the response of the length compensation elements. When one bends body section 1615, one side of length compensation section 1617 is activated by a pull force in the guiding elements, whereas the opposing side of length compensation section 1617 is activated by a push force in the opposing guide element. If there is difference in the magnitude in the forces, or a difference in movement of Bowden cable elements 1619(2,i), 1621(2,i) due to their length, plays, buckling effects, etc. it is better to directly couple the deformation of Bowden cable elements 1619(2,i), 1621(2,i) at one side of length compensation section 1617 to Bowden cable elements 1619(2,i), 1621(2,i) at the opposing side of length compensation section 1617. Of course many other mechanisms to couple or freeze the shape or the length of the length compensation section can be envisioned.

FIGS. 13-24b describe an instrument according to the invention in which a Bowden cable guide is built from portions of a separate first intermediate tube 1619 and a separate second intermediate tube 1621 and in which the steering wires 16(i) are made in a separate in between tube 1620. If one adds supportive inner tube 1601 and supportive outer tube 1602 that provides the structures (hinges) for the bendable sections, however, then one needs 5 tubes to build this instrument. A more efficient way is to make the steering wires 16(i) and the guidings therefore in a single tube. Embodiments of such an implementation are now explained.

FIG. 25A shows an embodiment of an instrument according to the invention in which the inner steering wire guiding portion 1619(2,1) and a substantial portion of steering wire 16(1) are made in one tube, i.e., first intermediate tube 1619. Third intermediate tube 1621 is not shown in FIG. 25A but in FIG. 25B.

In this embodiment inner, steering wire 16(1) has the following portions as viewed from the proximal end to the distal end: a flexible steering wire portion 16(1,2) in steering section 1618, a steering wire length compensation portion 16(1,3) in length compensation section 1617, a steering wire attachment portion 16(1,4) in the transition between length compensation section 1617 and body section 1615, and steering wire body section portion 16(1,5). Steering wire guiding portion 1619(2,1) and steering wire body section portion 16(1,5) are both made in intermediate tube 1619 whereas all other mentioned portions 16(1,2), 16(1,3) and 16(1,4) are made in tube 1620.

Steering wire attachment portion 16(1,4) is provided with an opening 1648 in which a sliding element 1647 is located. This sliding element 1647 is attached to first intermediate tube 1619, e.g. by laser welding, gluing, etc such that steering wire attachment portion 16(1,4) can slide along sliding element 1647 in the longitudinal direction. Moreover, steering wire attachment portion 16(1,4) is attached to steering wire body section portion 16(1,5). This attachment can be made with any suitable attachment method, preferably laser welding. In this way, any push or pull action exerted on flexible steering wire portion 16(1,2) is directly transferred to the same movement of steering wire body section portion 16(1,5) (and further to the tip section 1613).

The same construction holds for all steering wires 16(i).

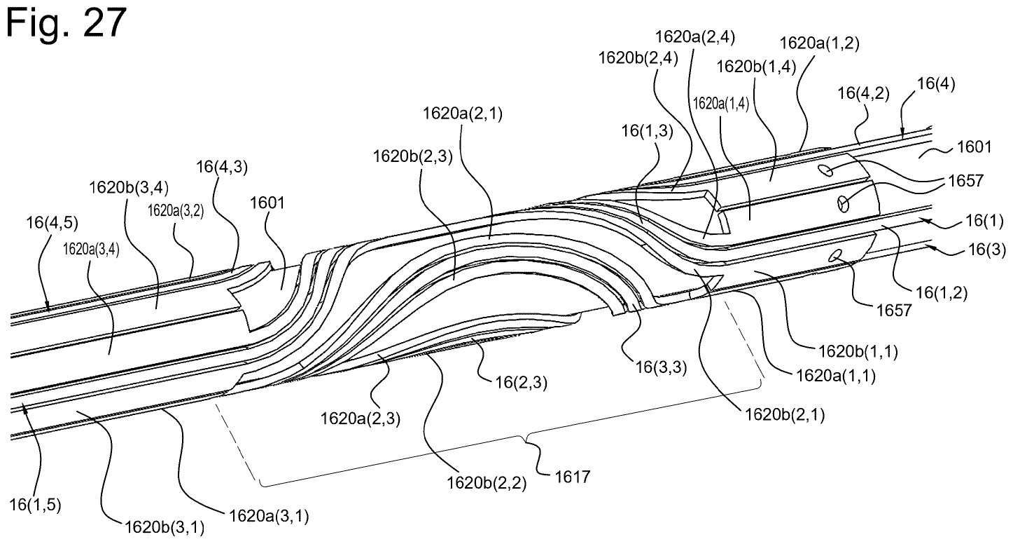

After the product of FIG. 25A is finished third intermediate tube 1621 containing the outer steering wire guiding portion 1621(2,1) is slid over this product, as shown in FIG. 26. Outer steering wire guiding portion 1621(2,1) is, at its distal end attached to sliding element 1647 at an attachment point 1655. This can, e.g., be done by laser welding, gluing, etc. Moreover, third intermediate tube 1621 is flexible at the longitudinal location where flexible steering wire portions 16(1,2) are located such as to make a bendable steering section 1618. This is, here, done by a suitable pattern of slots 1653.

FIG. 25B shows the finished assembly in which the complete instrument is made in three tubes 1619, 1620, 1621. First intermediate tube 1619 contains both inner steering wire guiding portions 1619(2,i) and body section steering wire portions 16(i,5). Third intermediate tube 1621 contains outer steering wire guiding portions 1621(2,i) and the further outer body structure.

One will understand that no extra inner tube 1601 or outer tube 1602 is needed anymore, though one of them or both may be applied as well.

FIGS. 26A-26F show an alternative to the embodiments of FIGS. 16-25B. The same reference numbers refer to the same or similar elements. Features explained with reference to FIGS. 16-25B which are not repeated in FIGS. 26A-26F can equally be applied in FIGS. 26A-26F unless such is physically impossible.

FIG. 26A shows a 3D side view in which no outer tube 1602 is present. In practice such an outer tube 1602 may be applied but that is not necessary. This system has a Bowden cable structure cut from tubes. The difference with the preceding expanding systems is that the structure is spiraled around the central axis of the instrument. The functionality is almost equal: if the instrument shaft is bent in the flexible body section 1615, control elements in the length compensation section 1617, at the same side as the inside of the bend in the instrument shaft will obtain a shorter path length and will bend outwards, away from the central axis. Steering elements 16(i) located at this inside of the bend in the flexible body section will have less longitudinal space inside the flexible body section but obtain more longitudinal space due to these control elements in the length compensation section.

Contrary, control elements in the length compensation section at the same side as the outside of the bend in the instrument shaft will obtain a longer path length and will be forced towards the central axis. Steering elements 16(i) located at this outside of the bend in the flexible body section will obtain more longitudinal space inside the flexible body section but obtain less longitudinal space due to these control elements in the length compensation section.

As a consequence, steering element length changes inside flexible body section 1615 due to bending are compensated by control elements inside the length compensation section 1617 and undesired steering of the articulating tip section 1613 is avoided.

FIG. 26A shows the outside of an embodiment of a length compensation section 1617 of an instrument made of five coaxially arranged tubes, i.e., inner tube 1601, extra intermediate tube 1608, first intermediate tube 1619, second intermediate tube 1620 and third intermediate tube 1621. Inner tube 1601 may be left out in some embodiments. The right hand side is the proximally located steering section 1618, the middle part is the length compensation section 1617 and to the left, a small detail of the flexible body section 1615 is shown. In this embodiment, the most right portion of the third intermediate tube 1621 inside steering section 1618 comprises a solid part without hinges in the form of a slot pattern. However, steering section 1618 may be flexible itself such that bending of steering section 1618 controls longitudinal movement of steering wires 16(i) and thus steering of tip section 1613, as explained with reference to FIGS. 1-10, and 18B.

This embodiment shows Bowden cable structures for four steering wires 16(i). However, this embodiment is not limited to such a number. The number may be one or more. In length compensation section 1617 four strip like, outside steering wire guiding portions 1621(2,i) are provided which are separated by slots. Each one of the outside steering wire guiding portions 1621(2,i) spirals about the central axis of the instrument. The amount of tangential spiraling from their proximal ends to their distal ends may be 360 degrees. However, the required amount of tangential spiraling depends on the specific application and may, thus, be more or less than 360 degrees. An adequate amount may be in a range of 10-1440 degrees, for instance in a range of 45-1080 degrees.

At their distal end, each one of the four outside steering wire guiding portions 1621(2,i) is attached to an outside steering wire guiding portion end part 1621(2,i)E. FIG. 26A shows that each one of these outside steering wire guiding portion end parts 1621(2,i)E is attached to portions of the second intermediate tube 1620 at two attachment points 1655a, 1655b, as will be explained in more detail hereinafter. Each one of these outside steering wire guiding portion end parts 1621(2,i)E is, in a rest condition (no bending of the flexible body section 1613), arranged such that it is at a distance from body section portion 1621(3). Thus, each one of them can move in the longitudinal direction to a certain designed extend, both in the proximal direction and in the distal direction, independently from all other outside steering wire guiding portion end parts 1621(2,i)E.

At the distal side of outside steering wire guiding portion end part 1621(2,i)E, FIG. 26A shows steering wires 16(i) for i=1 and i=3. Reference numbers 1601(1), 1601(3) refer to portions of inner tube 1601, as will become apparent hereinafter. Some portions of second intermediate tube 1620 and first intermediate tube 1619 are also visible. They will now be explained with reference to FIG. 26B which shows these portions in more detail.

FIG. 26B the same instrument as FIG. 26A, however, one in which third intermediate tube 1621 is not present. FIG. 26B shows portions of four steering wires 16(i). Looking into the distal direction, the tangential, clockwise order is i=1, 4, 2, 3. The tangential distance between the centers of two adjacent steering wires is 90 degrees. In steering section 1618, the space between steering wires 16(1,2), 16(4,2) is filled with a spacer 1620(1,4), the space between steering wires 16(4,2), 16(2,2) with a spacer 1620(1,2), the space between steering wires 16(2,2), 16(3,2) with a spacer 1620(1,3), and the space between steering wires 16(3,2), 16(1,2) with a spacer 1620(1,1). These spacers 1620(1,i) for i=1, 2, 3, 4 are separated from adjacent steering wire portions 16(i,2) by small slots such that they guide adjacent steering wires 16(i,2) in the longitudinal direction and prevent tangential movement of steering wires 16(i,2) in steering section 1618.

At the transition between steering section 1618 and length compensation section 1617, each spacer 1620(1,i) is attached to two longitudinal guiding element length compensation portions 1620a(2,i), 1620b(2,i). Inside length compensation section 1617, steering wire portion 16(1,3) is arranged between longitudinal guiding element length compensation portions 1620a(2,4) and 1620b(2,1), steering wire portion 16(4,3) between longitudinal guiding element length compensation portions 1620a(2,2) and 1620b(2,4), steering wire portion 16(2,3) between longitudinal guiding element length compensation portions 1620a(2,3) and 1620b(2,2), and steering wire portion 16(3,3) between longitudinal guiding element length compensation portions 1620a(2,1) and 1620b(2,3).

Sets of longitudinal guiding element length compensation portions 1620a(2,4)/1620b(2,1), 1620a(2,2)/1620b(2,4), 1620a(2,3)/1620b(2,2), and 1620a(2,1)/1620b(2,3), respectively, are separated from steering wire portion 16(1,3), 16(4,3), 16(2,3), and 16(3,3), respectively, by small slots such that they guide steering wire portion 16(1,3), 16(4,3), 16(2,3), and 16(3,3), respectively in the longitudinal direction and prevent independent tangential movement of steering wire 16(1,3), 16(4,3), 16(2,3), and 16(3,3), respectively in length compensation section 1617.

Towards the distal end, each one two longitudinal guiding element length compensation portions 1620a(2,i), and 1620b(2,i), respectively, is attached to an end portion 1620a(2,i)E, and 1620b(2,i)E, respectively. The two end portions 1620a(2,i)E and 1620b(2,i)E are separated by a slot such that they can move in the longitudinal direction independently. In an embodiment, the slot is configured such that the two end portions 1620a(2,i)E and 1620b(2,i)E prevent mutual tangential movement.

All end portions 1620a(2,i)E, 1620b(2,i)E, in a rest situation, are arranged such that there is a free space towards the distal direction allowing them to be movable in the distal direction. In the shown embodiment, this free space ends at the transition between length compensation section 1617 and flexible body section 1615. Portions of the steering wires 16(i) inside this free space are indicated with reference numbers 16(i,4), whereas the portions of steering wires 16(i) in flexible body section 1615 are indicated with reference numbers 16(i,5). In the shown embodiment, the steering wire portions 16(i,5) are wider at the transition between length compensation section 1617 and flexible body section 1615 than inside that free space, however, that is not required.

In the assembled state of the present embodiment, third intermediate tube 1621 is present outside second intermediate tube 1620, each one of the outside steering wire guiding portions 1621(2,i) is aligned with one steering wire portion 16(i,3) such that it covers that steering wire portion 16(i,3) along its entire length and prevents any radial movement of that steering wire portion 16(i,3) independently from radial movement of outside steering wire guiding portion 1621(2,i).

In an embodiment, to further prevent independent tangential movement of steering wire portions 16(1,3), 16(4,3), 16(2,3), and 16(3,3), respectively, each set of end portions 1620a(2,4)E/1620b(2,1)E, 1620a(2,2)E/1620b(2,4)E, 1620a(2,3)E/1620b(2,2)E, and 1620a(2,1)E/1620b(2,3)E, respectively, of each set of longitudinal guiding element length compensation portions 1620a(2,4)/1620b(2,1), 1620a(2,2)/1620b(2,4), 1620a(2,3)/1620b(2,2), and 1620a(2,1)/1620b(2,3), respectively, is attached to one end portion 1621(2,1)E, 1621(2,3)E, 1621(2,2)E, and 1621(2,4)E, respectively, at attachment locations 1655a, 1655b (cf. FIG. 26A). Thus, the end portions of each set of end portions 1620a(2,4)E/1620b(2,1)E, 1620a(2,2)E/1620b(2,4)E, 1620a(2,3)E/1620b(2,2)E, and 1620a(2,1)E/1620b(2,3)E, respectively, are fixed in mutual tangential and longitudinal movement.

The pattern of slots as provided in first intermediate tube 1619, and as shown in FIG. 26C, is quite similar to the one in third intermediate tube 1621, as shown in FIG. 26A.

In this embodiment, cf. FIG. 26C, the most right portion of the first intermediate tube 1619 inside steering section 1618 comprises a solid part. It may have one or more slotted patterns, e.g. to form hinges.

In length compensation section 1617 four strip like inside steering wire guiding portions 1619(2,i) are provided which are separated by slots. Each one of the inside steering wire guiding portions 1619(2,i) spirals about the central axis of the instrument, in the same way as do outside steering wire guiding portions 1621(2,i) and steering wire portions 16(2,i).

At their distal end, each one of the four inside steering wire guiding portions 1619(2,i) is attached to an inside steering wire guiding portion end part 1619(2,i)E.

Each set of end portions 1620a(2,4)E/1620b(2,1)E, 1620a(2,2)E/1620b(2,4)E, 1620a(2,3)E/1620b(2,2)E, and 1620a(2,1)E/1620b(2,3)E, respectively, of each set of longitudinal guiding element length compensation portions 1620a(2,4)/1620b(2,1), 1620a(2,2)/1620b(2,4), 1620a(2,3)/1620b(2,2), and 1620a(2,1)/1620b(2,3), respectively, in second intermediate tube 1620 (cf. FIG. 26B) is attached to one end portion 1619(2,1)E, 1619(2,3)E, 16191(2,2)E, and 1619(2,4)E, respectively, at attachment locations 1656. Thus, the end portions of each set of end portions 1620a(2,4)E/1620b(2,1)E, 1620a(2,2)E/1620b(2,4)E, 1620a(2,3)E/1620b(2,2)E, and 1620a(2,1)E/1620b(2,3)E, respectively, are fixed in mutual tangential and longitudinal movement.

Each one of these inside steering wire guiding portion end parts 1619(2,i)E is, in a rest condition (i.e., no bending of the flexible body section 1613), arranged such that it is at a distance from flexible body section 1615. Thus, each one of them can move in the longitudinal direction to a certain designed extent, both in the proximal direction and in the distal direction, independently from all other inside steering wire guiding portion end parts 1619(2,i)E.

Turning now to FIG. 26D, a portion of inner tube 1601 is shown, inserted in an extra intermediate tube 1608. In the assembled state, they are located inside first intermediate tube 1619. At the proximal end, i.e., in the steering section 1618, extra intermediate tube 1608 comprises an extra intermediate tube portion which may be solid or provided with a suitable slotted pattern, e.g., for a hinge, depending on the required functionality of that portion. At a proximal side of the length compensation section 1617, in this embodiment, no parts of extra intermediate tube 1608 are present. At a distal side of the length compensation section 1617, extra intermediate tube 1608 comprises four (in this embodiment, as many as there are steering wires 16(i)) extra intermediate tube shiftable portions 1608(i). These extra intermediate tube shiftable portions 1608(i) run, in this embodiment, towards the proximal end of the tip where they are attached such that cannot move in the longitudinal direction at the tip section 1613. Adjacent extra intermediate tube shiftable portions 1608(i) are separated by slots such that they can shift along inner tube 1601 back and forth in the longitudinal direction. These slots are, preferably manufactured as small as possible to avoid tangential play between them. Inside flexible body section 1615, extra intermediate tube 1608 may comprise steering wire portions attached to, e.g., steering wire portions 16(1,5).

Referring also to FIG. 26D, each one of the inside steering wire guiding portion end parts 1619(2,i)E is attached to one such extra intermediate tube shiftable portion 1608(i), e.g., at an attachment location 1673.

As shown in FIGS. 26A-26C, each outside steering wire guiding portion 1621(2,i) has a width along its length such that it covers an outside surface of a respective steering wire portion 16(2,i) as seen in the radial direction. Moreover, each inside steering wire guiding portion 1619(2,i) has a width along its length such that it covers an inside surface of a respective steering wire portion 16(2,i) as seen in the radial direction.

The width of outside steering wire guiding portion 1621(2,1) 1621(2,4), 1621(2,2), and 1621(2,3), respectively, may be equal to the width of steering wire portion 16(1,3) plus the width of the set of longitudinal guiding element t length compensation portions 1620a(2,4)/1620b(2,1), the width of steering wire portion 16(4,3) plus the width of the set of longitudinal guiding element length compensation portions 1620a(2,2)/1620b(2,4), the width of steering wire portion 16(2,3) plus the width of the set of longitudinal guiding element length compensation portions 1620a(2,3)/1620b(2,2), and the width of steering wire portion 16(3,3) plus the width of the set of longitudinal guiding element length compensation portions 1620a(2,1)/1620b(2,3), respectively.

Moreover, the width of inner steering wire guiding portions 1619(2,1) 1619(2,4), 1619(2,2), and 1619(2,3), respectively, may be equal to the width of outer steering wire guiding portions 1621(2,1) 1621(2,4), 1621(2,2), and 1621(2,3), respectively.

As a consequence, each steering wire portion 16(i,3) is located inside a channel in length compensation section 1617. I.e., steering wire portion 16(1,3) is located in a channel formed by inner steering wire guiding portions 1619(2,1), longitudinal guiding element length compensation portions 1620a(2,4)/1620b(2,1), and outside steering wire guiding portion 1621(2,1). Steering wire portion 16(4,3) is located in a channel formed by inner steering wire guiding portions 1619(2,4), longitudinal guiding element length compensation portions 1620a(2,2)/1620b(2,4), and outside steering wire guiding portion 1621(2,4). Steering wire portion 16(2,3) is located in a channel formed by inner steering wire guiding portions 1619(2,2), longitudinal guiding element length compensation portions 1620a(2,3)/1620b(2,2), and outside steering wire guiding portion 1621(2,2). Steering wire portion 16(3,3) is located in a channel formed by inner steering wire guiding portions 1619(2,3), longitudinal guiding element length compensation portions 1620a(2,1)/1620b(2,3), and outside steering wire guiding portion 1621(2,3).

Length compensation section 1617 of the embodiment of FIGS. 26A-26D functions in the following way. This will be explained with reference to steering wires 16(1), 16(2) which are oppositely located in the present embodiment. The explanation is the same for other opposite steering wires. Reference is made to FIGS. 26E (side view) and 26F (perspective view) in which the instrument is slightly rotated in the tangential direction relative to FIGS. 26A-26D.

Assume flexible body section 1615 is bent such that the proximal end of extra intermediate tube shiftable portion 1608(1) is pushed towards the proximal end. Then, the proximal end of extra intermediate tube shiftable portion 1608(1), inner steering wire guiding portion end part 1619(2,1)E, the set of end portions 1620a(2,4) E/1620b(2,1)E of longitudinal guiding element length compensation portions 1620a(2,4)/1620b(2,1), and outer guiding steering wire guiding portion end part 1621(2,1)E slide towards the proximal end because they are all attached. This causes the spiraling part of inner steering wire guiding portion 1619(2,1), longitudinal guiding element length compensation portions 1620a(2,4)/1620b(2,1), and outside steering wire guiding portion 1621(2,1) in the length compensation section 1617 to be moved away from the central axis. This causes more path length to be available for steering wire 16(1) in length compensation section 1617 which is necessary to compensate for steering wire portion 16(1,5) longitudinally moving into length compensation section 1617 to a certain extent because of the bending of the flexible body section 1615.

Because of the bending of the flexible body portion 1615 oppositely located proximal end of extra intermediate tube shiftable portion 1608(2) is pulled towards the distal end. Then, the proximal end of extra intermediate tube shiftable portions 1608(2), inner steering wire guiding portion end part 1619(2,2)E, the set of end portions 1620a(2,3)E/1620b(2,2)E of longitudinal guiding element length compensation portions 1620a(2,3)/1620b(2,2), and outer guiding steering wire guiding portion end part 1621(2,2)E slide towards the distal end because they are all attached. This causes the spiraling part of inner steering wire guiding portion 1619(2,2), longitudinal guiding element length compensation portions 1620a(2,3)/1620b(2,2), and outside steering wire guiding portion 1621(2,2) in the length compensation section 1617 to be moved towards the central axis. This causes less path length to be available for steering wire 16(2) in length compensation section 1617 which is necessary to compensate for steering wire portion 16(2,4) longitudinally moving into flexible body section 1615 to a certain extent because of the bending of the flexible body section 1615.

The bending inside flexible body section 1615 may be such that steering wire portions 16(3,4)/16(3,3) and 16(4,4)/16(4,3) do not move at all and need no length compensation. However, depending on the direction of that bending they may also move which will be compensated in a similar way in length compensation section 1617.

Thus, all movements of the steering wire portions 16(i,5) inside flexible body section 1615 due to bending of that section is compensated by the Bowden cable structure inside length compensation section 1617, and the tip section 1613 is not bent inadvertently.

Of course one can envision more embodiments in which body structures, steering wires and Bowden cable elements are combined in tubes with the intention to reduce the number of required tubes and to make assembly of such an instrument as easy as possible.

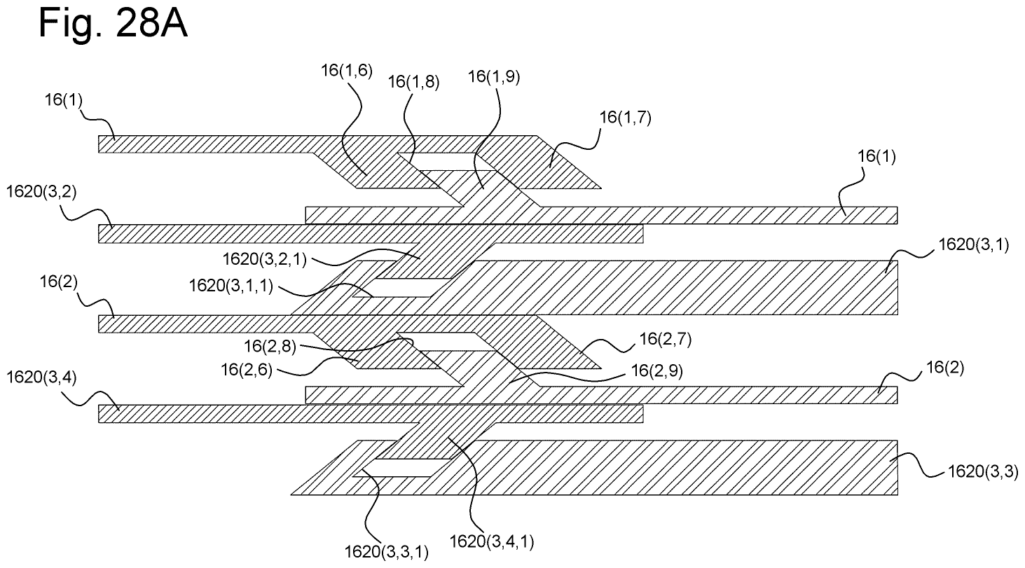

FIG. 27 shows an embodiment in which the compressible or stretchable curvy shape of length compensation section 1617 can be made in the tangential direction of the instrument without increase of the instrument overall diameter and without the necessity of a separate shaping process. In this embodiment, no separate construction of a steering wire guiding is required. The steering wires 16(i) are guided by an inner and outer tube in the radial direction and by longitudinal guiding elements in the tangential direction, which lye adjacent to the steering wire 16(i). One can also envision that many other shapes are possible as long as the shape is longitudinally compressible or stretchable and as long as friction on the steering wire 16(i) is kept to an acceptable level. This latter observation is also applicable to radially formed shapes as shown in FIGS. 13-26F.

In the implementation example of FIG. 27, only parts of inner tube 1601 and intermediate tube 1620 are shown. Outer tube 1621 is not shown. Like in FIGS. 26A-26F, inside length compensation section 1617, steering wire portion 16(1,3) is arranged between longitudinal guiding element length compensation portions 1620a(2,4) and 1620b(2,1), steering wire portion 16(4,3) between longitudinal guiding element length compensation portions 1620a(2,2) and 1620b(2,4), steering wire portion 16(2,3) between longitudinal guiding element length compensation portions 1620a(2,3) and 1620b(2,2), and steering wire portion 16(3,3) between longitudinal guiding element length compensation portions 1620a(2,1) and 1620b(2,3). These longitudinal guiding element length compensation portions can be coupled by a bridging element in the inner tube 1601 or outer tube 1602 to create a more defined channel. Each steering wire 16(i) has a steering wire steering section portion 16(i,2), a steering wire length compensation section portion 16(i,3) and a steering wire body section portion 16(i,5) (their tip portions are not shown but may be equal to the other embodiments). Here, each spacer 1620(1,i) as shown in the transition between length compensation section 1617 and steering section 1618 of FIG. 26B is separated into two portions 1620a(1,i), 1620b(1,i) which are both attached to inner tube 1601 or outer tube 1621 or both by, e.g., laser welding, gluing, etc. at location 1657. Such splitting is not necessary.

In the body section 1615, steering wire portion 16(1,5) is arranged between longitudinal guiding element body portions 1620a(3,4) and 1620b(3,1), steering wire portion 16(4,5) between longitudinal guiding element body portions 1620a(3,2) and 1620b(3,4), steering wire portion 16(2,5) between longitudinal guiding element body portions 1620a(3,3) and 1620b(3,2), and steering wire portion 16(3,5) between longitudinal guiding element body portions 1620a(3,1) and 1620b(3,3). These latter longitudinal guiding element body portions run to the transition between the body section 1615 and tip section 1613 and are attached there to the inner tube 1601 or outer tube 1602 or both by, e.g., laser welding, gluing, etc. to prevent longitudinal motion at that location. The embodiment of FIG. 27 operates, essentially, in the same way as the one of FIGS. 16-26F, be it that each first portion of a steering wire (16(i)) and associated steering wire guiding portions are here configured as a length compensation element with a curved configuration in the tangential direction only, which is configured to deform to absorb a length change of a second portion of the steering wire (16(i)) inside the flexible body section (1615) due to bending of the flexible body section (1615).

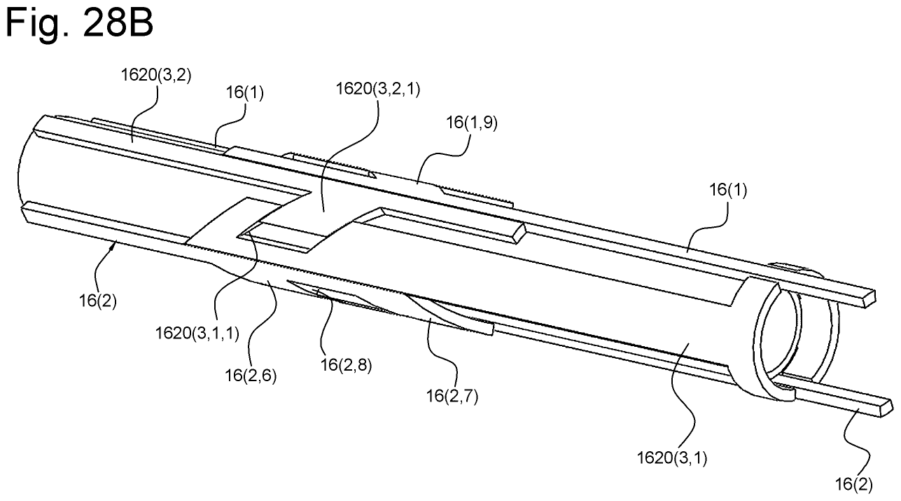

FIGS. 28a and 28b show an embodiment of a length compensation section 1617 that can be built in one tube 1620 and that does not require forming in a radial way. In fact, this length compensation section 1617 can be fully enclosed by a single inner and a single outer tube 1619, 1621 as in the embodiment of FIG. 27. FIG. 28a shows a flat representation and FIG. 28b shows the ‘as cut’ 3D presentation. These figures show an embodiment with two steering wires 16(1), 16(2) located at 180 degrees rotated relative to one another. However, if the diameter of the tubes is large enough, more than two steering wires may be applied. Also, an embodiment with only one steering wire can be made.

Each one of the steering wires 16(i) is split into two portions, i.e., a first portion and a second portion. The first portion is provided with a protrusion 16(i,9) extending in the tangential direction at a predetermined angle<90 degrees but >0 degrees. For instance, 30 degrees<angle<80 degrees. The second portion, which is attached to the distal end of the tip section 1613, has a recess 16(i,8), e.g., located between two extensions 16(i,6) and 16(i,7). The recess 16(i,8) is shaped to receive protrusion 16(i,9) in a slidable way. To that end, in an embodiment, recess 16(i,8) has an identical form as protrusion 16(i,9), i.e., is also extending in the tangential direction at the same angle.

In between the steering wires 16(i), steering wire guiding elements are provided. These steering wire guiding elements are attached to the body of the instrument. In the shown embodiment, they are located 90 degrees rotated from the steering wires 16(i) in the tangential direction. These steering wire guiding elements are grouped in sets of two steering wire guiding elements. Each set has a first steering wire guiding element 1620(3,1), 1620(3,3) and a second steering wire guiding element 1620(3,2) and 1620(3,4). Each first steering wire guiding element 1620(3,1), 1620(3,3) has a recess 1620(3,1,1), 1620(3,3,1). Each second steering wire guiding element 1620(3,2), 1620(3,4) has a protrusion 1620(3,2,1), 1620(3,4,1) which is received in the recess 1620(3,1,1), 1620(3,3,1) of first steering wire guiding element 1620(3,1), 1620(3,3). Both protrusion 1620(3,2,1), 1620(3,4,1) and recess 1620(3,1,1), 1620(3,3,1) are extending in the tangential direction at a predetermined angle<90 degrees but >0 degrees. For instance, 30 degrees<angle<80 degrees. These angles may be the same as applied in the steering wire protrusions 16(i,9) and steering wire recesses 16(i,8).

First steering wire guiding elements 1620(3,1) and 1620(3,3) are connected to the body of the instrument in the area of the proximal end of the steerable tip section. Second steering wire guiding elements 1620(3,2) and 1620(3,4) are connected to suitable portions of the body of the instrument too.

When the body section 1615 of the instrument of FIGS. 28a, 28b is bent, the proximal end of first steering wire guiding elements 1620(3,1) and 1620(3,3) will longitudinally displace over a certain length. Because of this displacement, the tilted protrusion 1620(3,2,1), 1620(3,4,1) will tangentially slide in or out the tilted recess 1620(3,1,1), 1620(3,3,1). Thus, second steering wire guiding element 1620(3,2), 1620(3,4) will move tangentially. In this movement it will also tangentially displace a first portion of steering wire 16(i) that is attached longitudinally to the steering device at the proximal end of the instrument. When the first portion of steering wire 16(i) that is attached to the steering device is displaced tangentially, it will longitudinally displace the second portion of steering wire 16(i). By properly selecting the tangential tilt angles of the protrusions and recesses of all elements, the length of change of steering wire 16(i) may be compensated such that the tip section 1613 does not deflect due to bending of the body section 1615. The construction now works as a length compensation element.

Yet, if one moves the steering wires in a longitudinal direction such as to deflect the tip section 1613 in a desired way this is not affected by the shown construction. Longitudinal movement of the first portion of steering wire 16(i) is transferred to a same longitudinal movement of the second portion of steering wire 16(i).

FIGS. 28a, 28b describe an embodiment of a sliding mechanism that copies a longitudinal displacement of a passive steering wire end in exactly the same amount and direction to a steering wire end. Of course other sliding or lever mechanisms can be envisioned.

FIGS. 27, 28a/28b show only two possible embodiments in which the length compensation section 1617 is cut in a single tube 1620. One can envision that more shapes are possible and that also length compensation sections can be build making use of more than one tube.

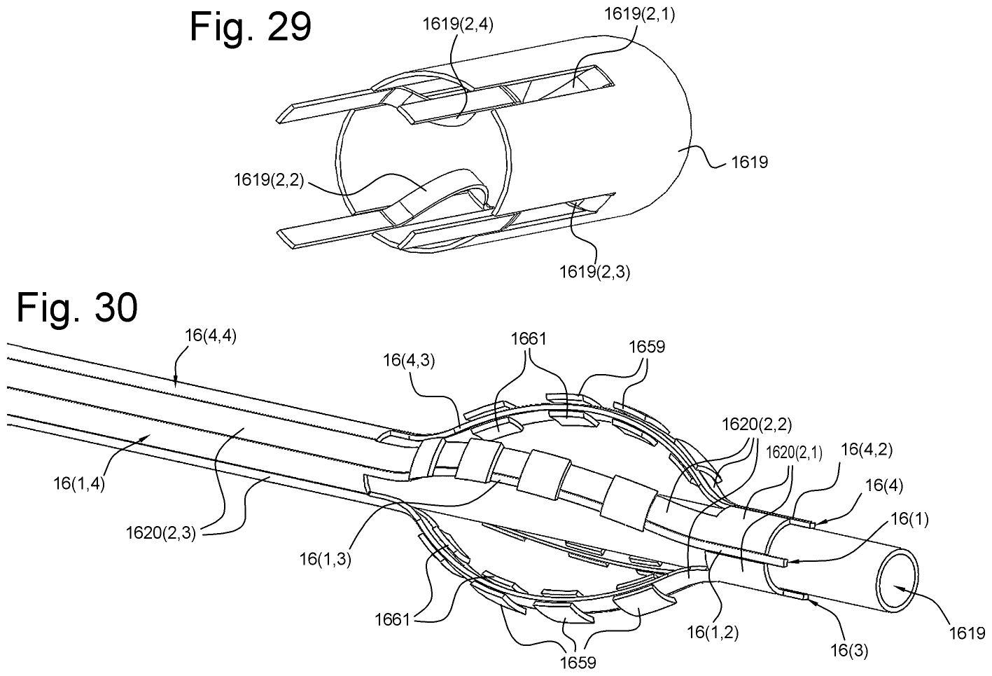

FIG. 29 shows an embodiment in which the Bowden cable arrangement, in the length compensation section 1617, is extending in the radially inward direction toward central axis 1622. For the sake of clarity, FIG. 29 only shows tube 1619 and its length compensation portions 1619(2,i). The other tubes 1620 and 1621 have a similar design in length compensation section 1617. Moreover, inner tube 1601 and outer tube 1602 may be applied as well. In order to provide enough inner space for each set of elements 1619(2,i), 16(i) and 1621(2,i) inside length compensation section 1617, adjacent such sets 1619(2,i), 16(i) and 1621(2,i) in the tangential direction may be, as shown, shifted along a certain longitudinal distance such that they cannot touch one another when bending inside to the central axis 1622.

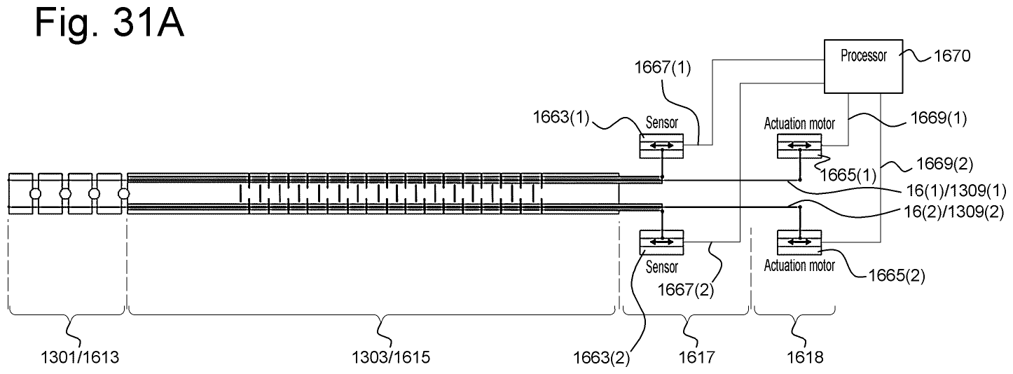

FIG. 30 shows an embodiment in which has at least one longitudinal guiding element length compensation portion 1620(2,2) is tangentially located adjacent to each steering wire 16(i) in length compensation section 1617. The elements in the length compensation section bend radially outward. The figure shows one such longitudinal guiding element length compensation portion 1620(2,2) for each steering wire 16(i) but there may be one at either side. Each longitudinal guiding element length compensation portion 1620(2,2) is cut from the same tube 1620 as from which steering wires 16(i) are cut. They are separated from one another by a small slot which may be as small as resulting from the smallest possible laser beam used to make the slot.

Each longitudinal guiding element length compensation portion 1620(2,2) is a portion of a longitudinal guiding element 1620(2,1), 1620(2,2), 1620(2,3). At the transition between length compensation section 1617 and steering section 1618, each steering wire steering section portion 16(i,2) is tangentially located between two adjacent proximal longitudinal guiding element portions 1620(2,1) which are attached to inner tube 1619 or outer tube 1621 or both by, e.g., laser welding, gluing, etc. In the body section 1615, each steering wire body section portion 16(i,4) is tangentially located between two adjacent longitudinal guiding element body portions 1620(2,3) which run to the transition between the body section 1615 and tip section 1613 and are attached there to the inner tube 1619 or outer tube 1621 or both by, e.g., laser welding, gluing, etc. to prevent longitudinal motion at that location. Again, separation slots may be very small, i.e., as small as resulting from the smallest possible laser beam used to make the slot.

FIG. 30 also shows one or more cover plates 1659, 1661 in length compensation section 1617. I.e., one or more cover plates 1659, e.g., cut from outer tube 1621, cover steering wires 16(i) on their radial outside and are attached to an adjacent longitudinal guiding element length compensation portion 1620(2,2), e.g., by laser welding, gluing, etc. Moreover, one or more cover plates 1661, e.g., cut from inner tube 1619, cover steering wires 16(i) on their radial inside and are attached to an adjacent longitudinal guiding element length compensation portion 1620(2,2), e.g., by laser welding, gluing, etc. Thus, in length compensation section 1617 each steering wire 16(i) is guided by adjacent guiding element at at least three sides. Guiding at four sides may be implemented too.

FIGS. 31a thru 31c show different embodiments of an electro mechanical length compensation section 1617 which can be applied both in a steerable deflectable instrument with wires in the form of classic cables 1309(i) and steering wires 16(i) manufactured from a tube. In the latter case, the steerable deflectable instrument may be one of the embodiments as explained in the present document with reference to any one of the earlier FIGS. 13-27, 19, 30. Because of that, the tip section is indicated with both reference signs 1301 and 1613, and the body section is indicated with both reference signs 1303 and 1615. If designed for robotic use, length compensation section 1617 can be made an integral part of a robotic steering section which can be coupled to the steering wire guiding and the steering wires 16(i).

FIG. 31a shows an embodiment in which proximal ends of the steering wire guidings are coupled to sensors 1663(i). The sensors 1663(i) are connected to a processor 1670 to send respective sensor signals 1667(i) to processor 1670. These sensors 1663(i) measure magnitude and longitudinal direction of the movement of the proximal end of each steering wire guiding when the body section 1303, 1615 is bent. The respective sensor signals 1667(i) are indications of these movement magnitudes and movement directions. Processor 1670 generates a compensation signal 1669(i) for each one of a plurality of actuators 1665(i) in dependence on the sensor signals 1667(i). Each actuator 1665(i) is coupled to one steering wire 1309(i) / steering wire 16(i) such that each steering wire 1309(i)/steering wire 16(i) is moved in the same direction and along the same path length as the proximal end of the respective steering wire guiding as measured by sensor 1663(i). In this way each set of one sensor 1663(i) and one actuator 1665(i) functions as a length compensation unit for one steering wire 1309(i)/steering wire 16(i) and its associated steering wire guiding.

Alternatively, a set of separate processors is applied each one connected to one set of one sensor 1663(i) and one actuator 1665(i) to perform the above mentioned function. Each one of the set of separate processors can be either located close to or inside a respective sensor 1663(i) or close to or inside a respective actuator 1665(i).

Moreover, in this example, each actuator 1665(i) is configured to move its steering wire 1309(i)/steering wire 16(i) as controlled by a suitable actuator signal generated by processor 1670 to control deflection of the tip section 1301/1613. Processor 1670 can generate the compensation signal and actuation signal simultaneously. This can be useful for active steering of the tip section 1301/1613 while advancing the instrument through a curved channel, e.g., inside a human body.

Each applied processor is equipped with a central processing unit, CPU, connected to suitable memory units (RAM, ROM, EPROM, etc.) and to suitable input/output units. The memory units are storing suitable computer programs which, once loaded by the CPU, provide the CPU with the capacity to perform the required functions. Input units are configured to receive input signals, e.g. from sensors 1663(i) and send them to the CPU for further processing. Output units are configured to receive output signals from the CPU and transmit them to external devices like actuation motors 1665(i) and brakes 1671 (i).

FIG. 31b shows the same setup as FIG. 31a, but now each sensor 1663(i) is equipped with a brake device 1671(i) which is also coupled to processor 1670. Brake device is configured to either allow or block longitudinal movement of the respective steering wire guiding proximal end. Each brake device 1671(i) can be activated by processor 1670 with a suitable brake control signal at the moment that the processor 1670 generates actuation signals for the actuators 1665(i) to control deflecting of tip section 1301/1613 with steering wires 1309(i) / steering wires 16(i) but the body section 1303/1615 is not allowed to change its current bent or unbent status. In this way the steering wire guidings are then kept in a stationary position and, consequently, body section 1303/1615 is kept in its current, possibly curved position. Thus, activation of the steering wires 1309(i)/steering wire 16(i) does not result in steering of the body section 1303/1615.

FIG. 31c shows another embodiment of the system as shown in FIG. 31b, but now each actuator 1665(i) is mechanically coupled to a proximal end of its associated steering wire guiding and is configured to move with the steering wire guiding proximal end during body section bending and to keep the distance that steering wire 1309(i)/steering wire 16(i) is extending proximally outside its steering wire guiding constant as long as no control signal is received by the actuator 1665(i). In this embodiment, the mechanical unit containing the actuator 1665(i) can also be equipped with a brake device 1671 (i) as in FIG. 31b, that holds the actuator 1665(i) and, thus, steering wire guiding proximal end in a certain fixed position as long as actuation signals are received. As one can envision, it is also possible to replace the steering wire guiding with simpler elements like a simple wire that activates the sensor.

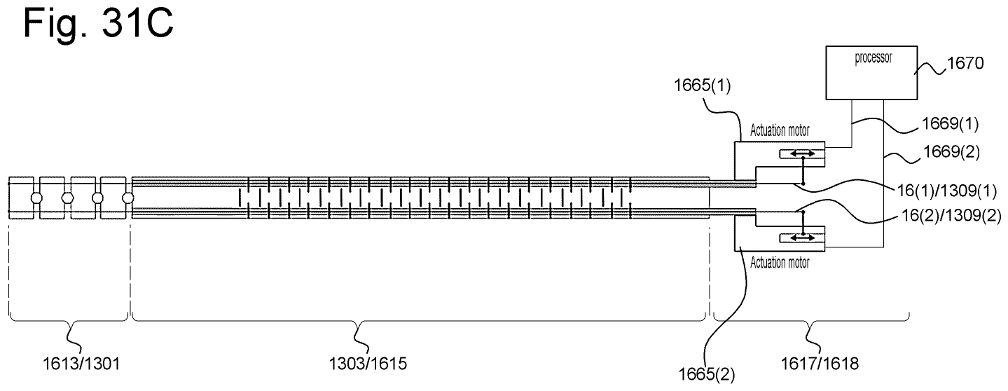

As explained in FIGS. 14 and 15, one can eliminate unwanted tip steering when the body of the instrument is bent and one can eliminate body steering when the tip is actively steered by a Bowden cable arrangement that incorporates a length compensation section. FIGS. 16 thru 27 show embodiments in which the length compensation section is based on such a Bowden cable arrangement and length compensation is established by deformation of the steering wire guiding element and the steering wires simultaneously in a radial or tangential way. As an alternative to deforming length compensation sections, FIGS. 28a and 28b describe an embodiment of a length compensation section with a sliding mechanism that copies a longitudinal displacement of a steering wire guiding element in exactly the same amount and direction to a steering wire end. Although useable, the shown solution has limitations with respect to obtainable length compensation. The larger the length compensation has to be, the larger the slope angle of the protrusions has to be. If the length compensation has to be relatively large, and the slope angle becomes more than 40 or 50 degrees, sliding frictions might become that high that the mechanism may not work anymore, or activation push or pull forces in the body length sensing elements become unacceptably high. Therefor an alternative sliding mechanism for a length compensation section is schematically presented hereinafter with reference to FIGS. 32-42E.

FIG. 32 shows a schematic setup of a length compensation section 3200 to explain the principles of the embodiments according to FIGS. 32-42H. This length compensation section 3200 is located at a location between a flexible body section (in FIG. 13 indicated with 1303) and the steering section (in FIG. 13 indicated with 1307) of the steerable instrument. FIG. 32 shows a body length sensing element 3201, which has the same function as the steering wire guiding element in the above embodiments of FIGS. 14 thru 27, except that this body length sensing element 3201 is merely used for body length sensing and does not have the main function of steering wire guiding. For this embodiment, it is assumed that the body length sensing element 3201 and the steering wire 16(i) are located in the same plane. Also, it is assumed that the body length sensing element 3201 and the steering wires 16(i) are guided by surrounding structures such that they only can move longitudinally and not in a vertical direction or in a direction perpendicular to the drawing plane.

In more detail, FIG. 32 shows a first wall 3202 extending in a transverse direction perpendicular to the longitudinal direction of steering wire 16(i) which is shown to have a first portion 16(i,1) and a second portion 16(i,2). Body length sensing element 3201 is implemented as a strip extending in the same longitudinal direction as steering wire 16(i). Length compensation section 3200 also comprises a second wall 3204 extending in in parallel to the first wall 3202, and a first slider 3218 which can slide up and down between walls 3202 and 3204 in the transverse direction as indicated with an arrow dV. First slider 3218 is provided with an opening 3203 accommodating a second slider 3219 such that second slider 3219 can slide back and forth in opening 3203 in a direction parallel to the longitudinal direction as indicated with an arrow dH.

Here, body length sensing element 3201 extends into the space between first and second walls 3202 and 3204 through a suitable opening in first wall 3202. Body length sensing element 3201 is provided with a protrusion 3206 extending in a slot 3212 inside first slider 3218. Here, slot 3212 is straight and extending at an angle 0<α1<90 degrees to the longitudinal direction.

First steering wire portion 16(i,1) also extends into the space between first and second walls 3202 and 3204 through a suitable opening in first wall 3202. First steering wire portion 16(i,1) is provided with a protrusion 3208 extending in a slot 3214 inside second slider 3219. Here, slot 3214 is straight and extending at an angle 0<α2<90 degrees to the transverse direction. Second steering wire portion 16(i,2) also extends into the space between first and

second walls 3202 and 3204 through a suitable opening in second wall 3204 such that first and second steering wire portions 16(i,1), 16(i,2) extend in opposite directions from length compensation section 3200. Second steering wire portion 16(i,2) is provided with a protrusion 3210 extending in a slot 3216 inside second slider 3219. Here, slot 3216 is straight and extending in the transverse direction.

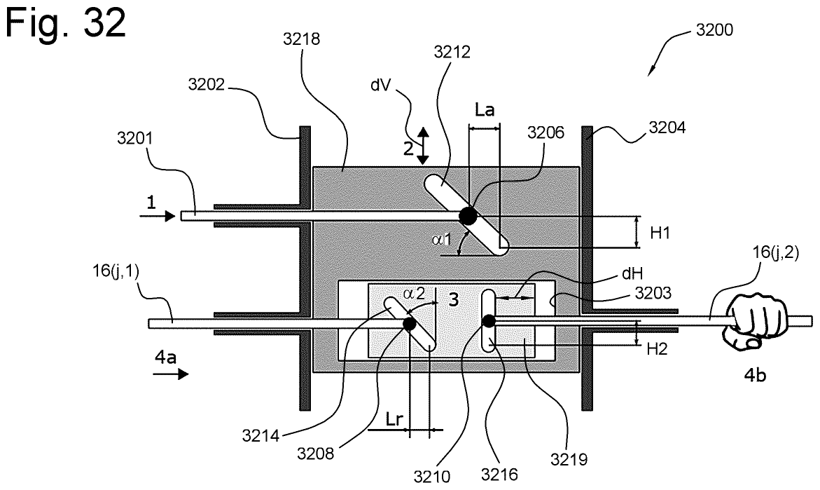

All protrusions 3206, 3208, 3210 can be implemented as a fixed protrusion either round or shaped in the geometry of the corresponding slot, or this can be for example a pin and wheel construction to reduce sliding friction, as shown in FIGS. 33A, 33B, 33C for protrusion 3206.

In FIG. 32, the body length sensing element 3201 is, for example, situated in an inner curve of a curved instrument body and therefor it got an over-length compared to a neutral line of the instrument body moving to the right hand direction in FIG. 32. Due to the angled slot 3212, first slider 3218 is than pushed in the transverse direction upwards over a distance H1 by the horizontal movement of body length sensing element 3201 over a distance La. This movement also moves second slider 3219, which is connected to first slider 3218 in the transverse direction but which can move freely in the longitudinal direction, upward over a distance H2, where H1=H2.

If second steering wire portion 16(i,2) is held stationary in place, for example by a (manual or robotic) steering input unit attached to second steering wire portion 16(i,2), second slider 3219 will not move in the longitudinal direction when it moves upward in the transverse direction, because slot 3216 in which steering wire 4b is connected, is extending in the transverse direction. Due to the other angled slot 3214 in second slider 3219, the end of first steering wire portion 16(i,1), attached to protrusion 3208 inside angled slot 3214, is displaced in the longitudinal direction over a distance Lr when second slider 3219 moves upward in the transverse direction together with first slider 3218. If the tilt angle 90-α2 of slot 3214 in second slider 3219 is the same as the tilt angle α1 of slot 3212 in first slider 3218, then the displacement Lr of first steering wire portion 16(i,1) is caused to be exactly the same as the displacement La of the body length sensing element 3201. When one now wants to steer the tip (e.g. located at the left hand side of the drawing of FIG. 32) of the instrument, one can pull or push second steering wire portion 16(i,2) in the longitudinal direction (e.g., manually or by a robotic device) and this movement then also pulls or pushes second slider 3219 in the longitudinal direction. Because first steering wire portion is also connected to second slider 3219, also first steering wire portion 16(i, 1) is pulled or pushed in the longitudinal direction with a same longitudinal displacement as second steering wire portion 16(i,2) and steering is accomplished.

It is observed that angle α1 may deviate from angle α2 such that displacements La and Lr may be different.

Internal frictions and activation forces in the length compensation section 3200 are strongly dependent on the tilt angles α1, α2 of the respective slots 3212, 3214. For example, if slot 3212 in first slider 3218 is close to the longitudinal direction one can understand that the activation force needed to move slider 3218 up with the body length sensing element 3201, is very low and the friction between protrusion 3206 and slot 3212 is also very low. If slot 3212 in first slider 3218 is close to the transverse direction, one can understand that one needs a very high activation force to move first slider 3218 up (or down) and that also the friction between protrusion 3206 and slot 3212 is very high. At the same time angle α2 should be minimized to allow protrusion 3208 to slide in slot 3214 with minimum friction once first and second sliders 3218, 3219 move up or down, but to prevent protrusion 3208 to slide easily in slot 3214 when one pulls or pushes second steering wire portion 16(i,2). So one can conclude that one must minimize tilt angles α1, and α2 as much as possible to keep frictions and activation forces at an acceptable level. In the above example, if one wants to minimize the tilt angles α1, α2 of both slots 3212, 3214 at given displacements La, Lr and H1, H2, these tilt angles must be around 45 degrees. A suitable design range for both of them would be between 35-55 degrees, preferably between 40-50 degrees.

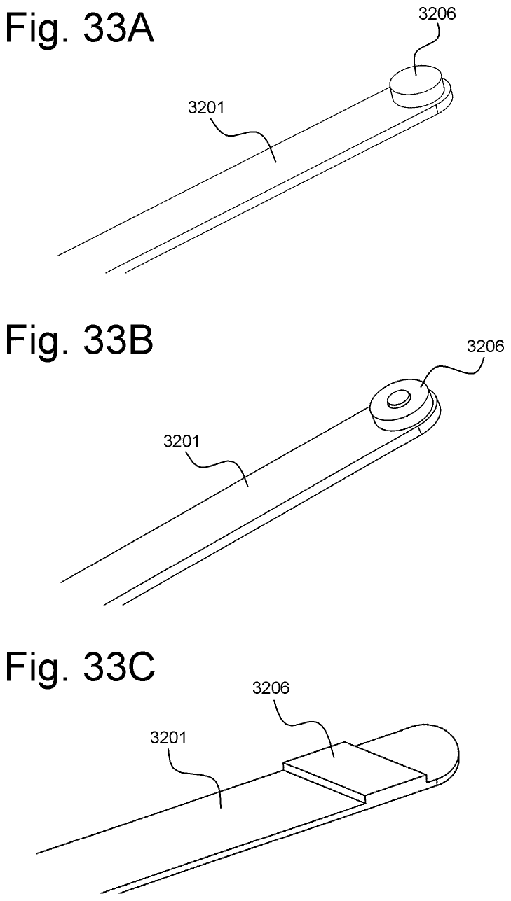

The mechanism of FIG. 32 can be further optimized with respect to frictions, activation forces and obtainable length compensation if one can minimize the tilt angle in combination with given length displacements La and Lr (Lr is preferably equal to La to obtain the correct length compensation), as shown in FIG. 34.

A difference between the embodiment of FIGS. 32 and 34 is that slot 3219 is no longer parallel to the transverse direction but extending at an angle 0<α3<90 degrees relative to the transverse direction. Angles α2 and α3 are directed in opposite directions relative to the transverse direction. Longitudinal displacement of first steering wire portion 16(i,1) and its protrusion 3208 is indicated with Lr1. Longitudinal displacement of second steering wire portion 16(i, 1) and its protrusion 3210 is indicated with Lr2.

In the above schematic solution of FIG. 34, one can minimize tilt angles α1, α2, α3 of all slots 3212, 3214, 3216 as long as Lr1 plus Lr2 is equal to La with a given transverse displacement H1=H2. In this mechanism, all slots 3212, 3214, 3216 now can have a smaller tilt angle α1, α2, α3 than 45 degrees. For each given La and H1=H2 one can calculate the optimal tilt angles α1, α2, α3 of all slots, including slot 3212 in slider 3218, when one knows the maximum allowable activation force on the body length sensing element 3201, friction coefficients and the required output force on second steering wire portion 16(i,2).

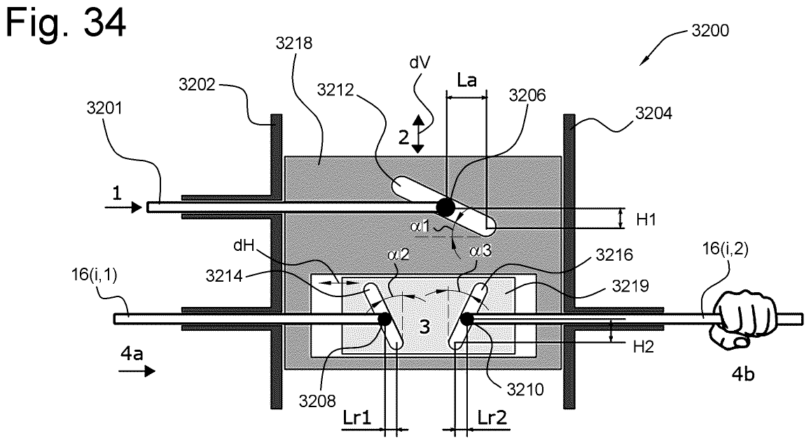

FIG. 35 shows an embodiment in which second slider 3219 is substituted by a third slider 3219a and a fourth slider 3219b which are both arranged such in first slider 3218 that they can move in the longitudinal direction but not in the transverse direction inside and relative to first slider 3218. These longitudinal displacements are respectively indicated by arrows dH1 and dH2.

Protrusion 3210 attached to second steering wire portion 16(i,2) extends in slot 3216 inside fourth slider 3219b. Here, slot 3216 is straight and extends at angle 0<α3<90 degrees to the transverse direction. Angles α2 and α3 are directed in opposite directions relative to the transverse direction.

Steering wire 16(i) is provided with a third steering portion 16(i,3) arranged between first steering portion 16(i, 1) and second steering wire portion 16(i,2) in the longitudinal direction. A first end of third wire portion 16(i,3) is attached to a protrusion 3220 extending in a slot 3222 inside third slider 3219a at an angle 0<α4<90 degrees. Angles α2 and α4 are directed in opposite directions relative to the transverse direction. A second end of third wire portion 16(i,3), opposite to the first end, is attached to a protrusion 3224 extending in a slot 3226 inside fourth slider 3219b at an angle 0<α5<90 degrees. Angles α3 and α5 are directed in opposite directions relative to the transverse direction.

The longitudinal displacement of first steering wire portion 16(i,1) and protrusion 3208 is indicated with Lr1. The longitudinal displacement of second steering wire portion 16(i,2) and protrusion 3210 is indicated with Lr2. The longitudinal displacement of third steering wire portion 16(i,3) and protrusion 3220 relative to third slider 3219a is indicated with Lr4 The longitudinal displacement of third steering wire portion 16(i,3) and protrusion 3224 relative to fourth slider 3219b is indicated with Lr3.

In the embodiment of FIG. 35, the required longitudinal length change Lr=Lr1+Lr2+Lr3+Lr4 of steering wire 16(i) is obtained by dividing longitudinal displacement La over four slots 3214, 3216, 3222, 3226 instead of two slots. In this case it is obvious that the tilt angles α1, α2, α3, α4, α5 of all slots 3212, 3214, 3216, 3222, 3226 can be further reduced, as long as Lr1+Lr2+Lr3+Lr4=La. One can also envision a mechanism with more than two sliders 3219a, 3219b to even further reduce the tilt angles.

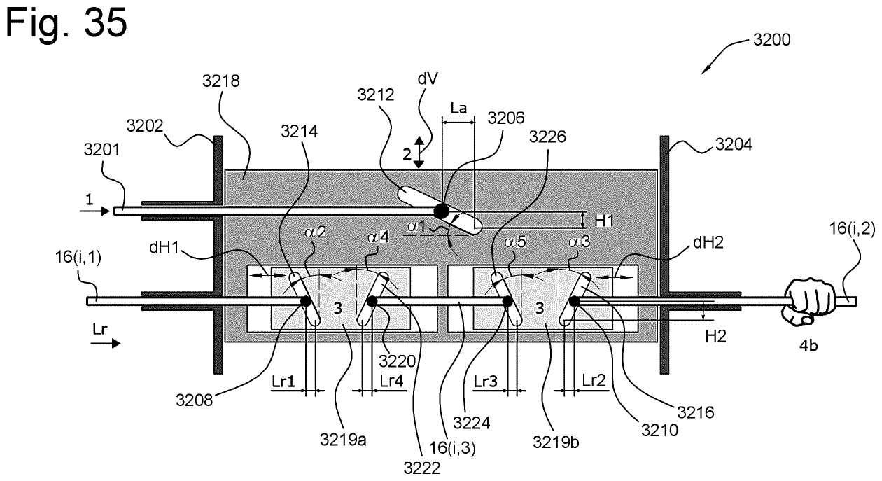

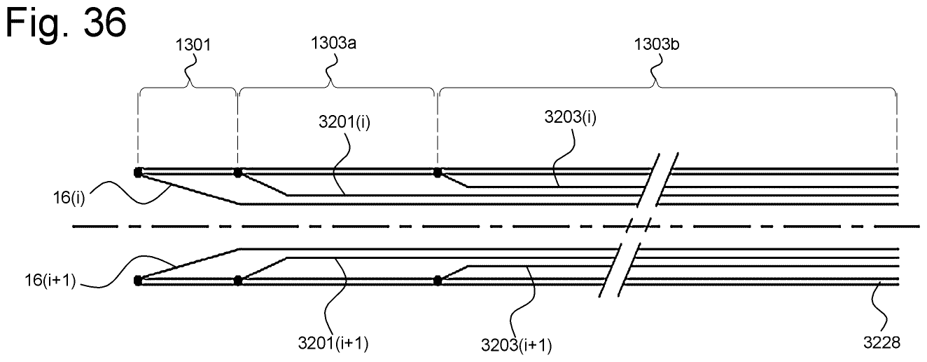

One can envision that one can have more than one group of body length sensing elements. In FIG. 36, two steering wires 16(i), 16(i+1) inside an outer tube 3228 are schematically shown. The instrument has a tip section 1301, a first body section 1303a, and a second body section 1303b. Two groups of sensing elements 3201(i)/3201(i+1), 3203(i)/3203(i+1) are shown. A first group of sensing elements 3201 (i), 3201(i+1) is attached in a location A in the distal part of the instrument body and a second group of sensing elements 3203(i), 3203(i+1) is attached in a location B. The steering wires 16(i) are attached in location C. Each group of sensing elements has its own length compensation section in the proximal part of the instrument. An example is shown in FIG. 37.

FIG. 37 shows a first length compensation section like the one shown in FIG. 34, as well as a second length compensation section. Second steering wire portion 16(i,2) is now substituted by third steering wire portion 16(i,3). Moreover, wall 3204 now also functions as a wall of the second length compensation section and one sensing element 3201 (i) of the first group extends into first slider 3218 like sensing element 3201 in FIG. 34.

Second length compensation section comprises a third wall 3230 extending in in parallel to the first and second walls 3202, 3204, and a third slider 3228 which can slide up and down between walls 3204 and 3230 in the transverse direction as indicated with an arrow dV2. Third slider 3228 is provided with an opening 3237 accommodating a fourth slider 3232 such that fourth slider 3232 can slide back and forth in opening 3237 in a direction parallel to the longitudinal direction.

Here, body length sensing element 3203(i) of the second group extends into the space between second and third walls 3204, 3230 through a suitable opening in second wall 3204. Body length sensing element 3203(i) is provided with a protrusion 3227 extending in a slot 3229 inside third slider 3228. Here, slot 3229 is straight and extending at an angle 0<α6<90 degrees to the longitudinal direction.

Third steering wire portion 16(i,3) also extends into the space between second and third walls 3204 and 3230 through a suitable opening in second wall 3204. Third steering wire portion 16(i,3) is provided with a protrusion 3236 extending in a slot 3234 inside fourth slider 3232. Here, slot 3216 is straight and extending at an angle 0<α8 <90 degrees to the transverse direction.

Second steering wire portion 16(i,2) also extends into the space between second and third walls 3204, 3230 through a suitable opening in third wall 3230 such that second and third steering wire portions 16(i,2), 16(i,3) extend in opposite directions from length compensation section 3200. Second steering wire portion 16(i,2) is provided with a protrusion 3240 extending in a slot 3238 inside fourth slider 3232. Here, slot 3238 is straight and extending at an angle 0<α7<90 degrees to the transverse direction.

The sensing elements 3203(i), 3203(i+1) of the second group sense the length change La2 of body section 1303b. This change has to be compensated fully so Lr2+Lr3=La2. The sensing elements 3201(i), 3201(i+1) of the first group sense the length change of body section 1303b plus the length change of body section 1303a. The length change of body section 1303b was already compensated by the mechanisms attached to the sensing elements 3203(i), 3203(i+1) of the second group and therefor does not have to be compensated anymore by the mechanism attached to sensing elements 3201(i), 3201(i+1) of the first group. The only compensation this mechanism has to generate is the compensation of body section 1303a plus a certain amount of over-compensation that forces the tip section 1301 in a bent configuration in the same direction as the bend in body section 1303a. So Lr1+Lr4=La1−La2+over-compensation.

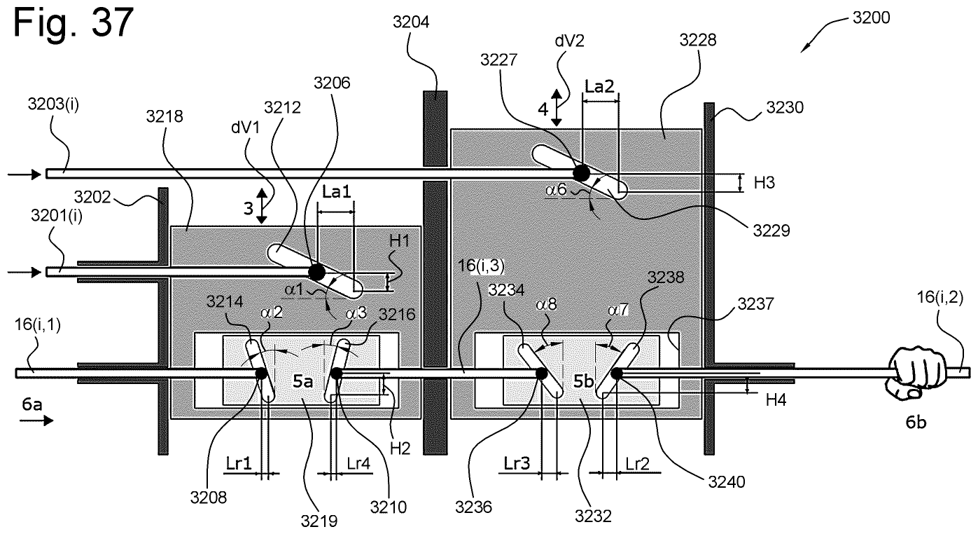

This embodiment might be useful in case of advancement of the instrument through a curved channel as depicted in FIG. 38A and 38B. During advancement through the mildly curved section of the channel it is no problem if the tip section 1301 of the instrument stays straight, which is accomplished by the length compensation section attached to sensing elements 3203(i), 3203(i+1) of the second group. When the tip section 1301 has to pass the tightly curved section of the channel, it would be advantageous if the tip section 1301 would automatically steer in the curved direction. This can be accomplished by a compensation section attached to sensing elements 3201 (i), 3201(i+1) of the first group, in which the length compensation unit so to say over-compensates and steers the tip section in the direction of the curve that was sensed by the sensing elements 3201(i), 3201(i+1) of the first group. One can envision that also more than two groups of sensing wires and corresponding length compensation sections can be used. Moreover, each group may have more than two sensing elements, like there may be more than two steering wires.

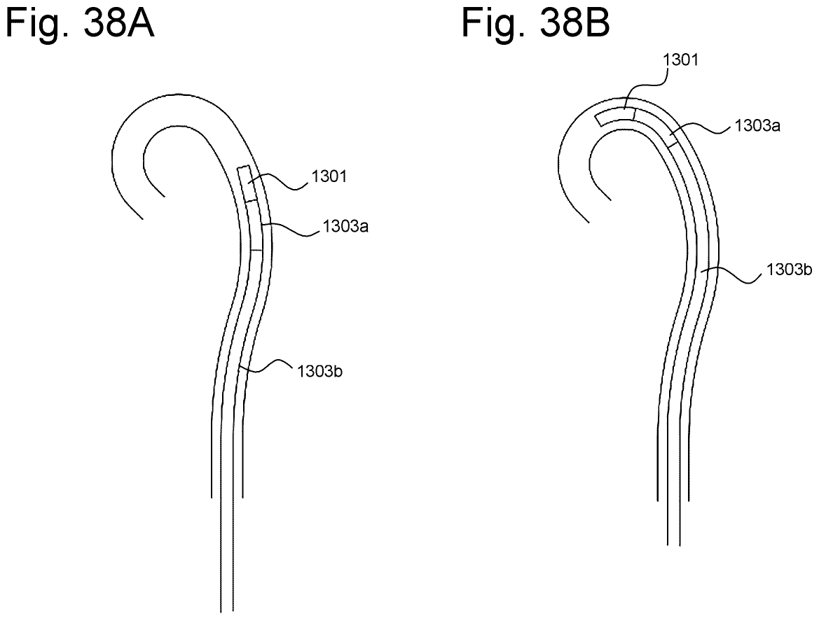

One can also envision that within the scope of the invention, more than one group of steering wires can be used and that the input signal of one set of body length sensing wires can be used to compensate the length of two sets of steering wires with which two steerable tip sections can be steered independently from each other. Cf. FIG. 39.

FIG. 39 comprises all elements of FIG. 34. Moreover, the embodiment of FIG. 39 comprises an identical section for a second steering wire 16(i+1) which is also divided in two separate portions extending in the longitudinal direction.

First portion 16(i+1,1) of the second steering wire also extends into the space between first and second walls 3202 and 3204 through a suitable opening in first wall 3202. First portion 16(i+1,1) is provided with a protrusion 3248 extending in a slot 3246 inside a third slider 3244. Here, slot 32146 is straight and extending at an angle 0<α9<90 degrees to the transverse direction. Third slider 3244 is arranged inside first slider 3218 such as to be movable inside first slider 3218 in the longitudinal direction independently from first slider 3218 but movable in the transverse direction only together with first slider 3218.

Second portion 16(i+1,2) of the second steering wire also extends into the space between first and second walls 3202 and 3204 through a suitable opening in second wall 3204 such that first and second portions 16(i+1,1), 16(i+1,2) extend in opposite directions from length compensation section 3200. Second portion 16(i+1,2) is provided with a protrusion 3252 extending in a slot 3250 inside second slider 3219. Here, slot 3250 is straight and extending at angle α10 to the transverse direction.

The mechanism now works the same for both steering wires 16(i) and 16(i+1) and does not need any further clarification here.

Like the mechanism of FIG. 34 is duplicated for more than one steering wire 16(i) in the embodiment of FIG. 39, this is also applicable to all embodiments of FIGS. 32, 35 and 37 as well.

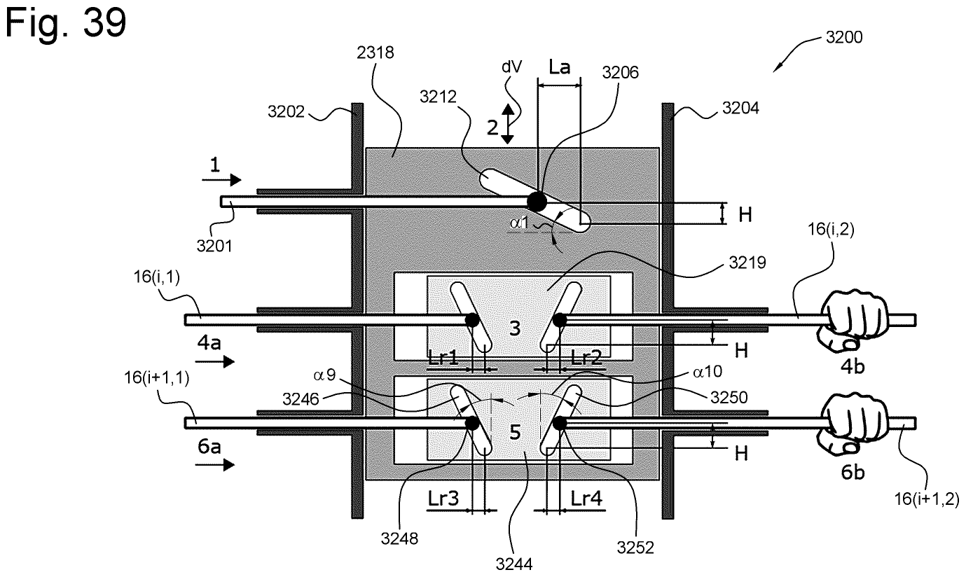

A practical problem may be that long instruments are often packed in a package with a hoop. Packaging a long instrument in a rolled up circular form results in a more compact and handier packing box shape than a very long, small and thin box. A problem with rolling up an instrument is that displacements of the body length sensing elements can be very high and if fact much higher than the displacements that are generated during normal use of an instrument. To prevent that the sensing elements break or buckle during rolling up, the compensation mechanism must be able to handle large length changes. In practice this can be a problem, because the allowable sliding capability of slider 3218 (and 3228) is limited. Another method is to absorb the length change of the body length sensing elements in another way as is depicted in FIG. 40.

In FIG. 40, an embodiment is shown almost identical to the one shown in FIG. 34. The difference is that slot 3212 is now provided with an end slot portion at both of its ends extending in the longitudinal direction.

The same principle can, of course be applied in all other shown and explained embodiments.

In this way, excessive length change of the body length sensing elements is possible, without increasing transverse displacement H to an impractical magnitude. Trade-off is that once longitudinal displacement La is reached, further displacement of the body length sensing element does not compensate the steering wires anymore and as a result of that the tip section will not be kept straight anymore. If the packaging hoop tube has a diameter that is large enough to accommodate a bent tip, this is not a problem.

In the above examples of FIGS. 32-40, the mechanism is presented in a flat plane. But, this mechanism can also be applied in a tubular shape. In a tubular instrument, the longitudinal movement is than in a direction equal to the longitudinal axis of the instrument, transverse movement is than equal to a tangential direction or a rotational direction around the longitudinal axis of the instrument and a movement perpendicular to the drawing plane is now in a radial direction, i.e. perpendicular to the longitudinal axis of the instrument.

The mechanism as explained with reference to FIGS. 32-40 can be applied in all steerable instrument embodiments of FIGS. 1-31, including ones that can be steered by manual operation or by a robotic steering device.

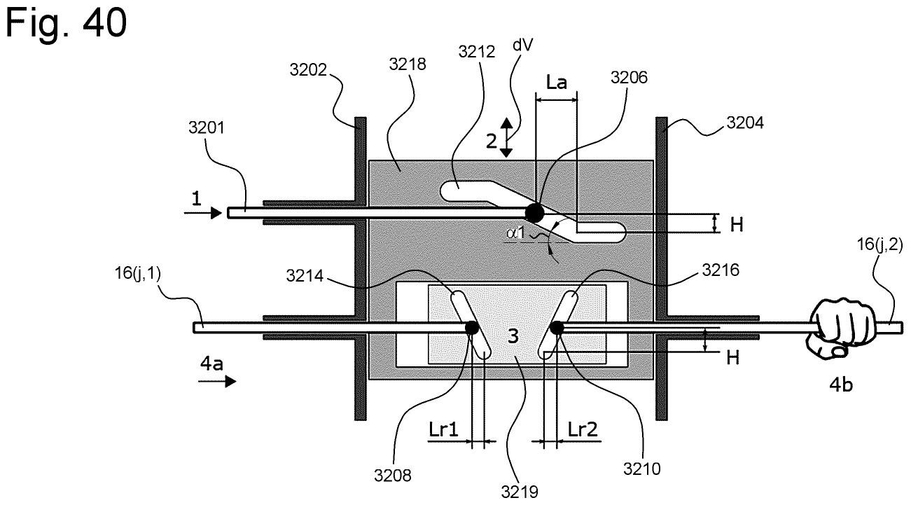

If one wants to apply a mechanism as presented in the previously shown FIGS. 32-40 in a tubular instrument and if the body length sensing element and the steering wire are in the same tubular wall, one has to bear the following in mind. If the body length sensing element is in a circumferential location in the tubular wall that is different than that of the steering wire, there is a difference in the displacement the body length sensing wire undergoes when the instrument body is bent and the length compensation the steering wire needs to keep the instrument tip straight. Cf. FIG. 41A in which steering wire 16(i) and sensing wire 3201 are drawn to be located in the same tube but at different tangential locations.

For example, as shown in FIG. 41A, when body length sensing element 3201 is situated 90 degrees separate from the circumferential location of steering wire 16(i), and the body is curved in a plane through the location of the steering wire 16(i), the body length sensing element end 3201 is not displaced because its curve length does not change. But, steering wire 16(i) would need a length compensation L to keep the tip straight. In this case, the mechanism would not work correctly. In practice, when the body length sensing element 3201 is right next to the steering wire the difference in body length sensing element end displacement is almost equal to the length compensation steering wire 16(i) would need and in practice, this difference is not noticeable in instrument performance.

However, one could overcome this problem when one would use a body length sense element for activation of first slider 3218 that displaces second slider 3219 with inversed slots that compensates the length of a steering wire 16(i) at a circumferential location exactly 180 degrees different from the location of body length sensing element 3201.

Another solution is to locate the body length sensing elements 3201 and the steering wires in different tube layers, as is shown in FIG. 41B. Now there still is a difference in length change of body length sensing element 3201 and the compensation that steering wire 16(i) needs, dependent on the radial location with respect to each other. For example when the steering wire is on top of the body length sensing element, as seen in the radial direction, steering wire 16(i) needs more length compensation than the length change of body length sensing element 3201. This can now easily be compensated by adjusting the tilt angles of the slots in the mechanism so that there is a fixed ratio between La and Lr equal to the ratio between the bending radii of sensing element 3201 and steering wire 16(i).

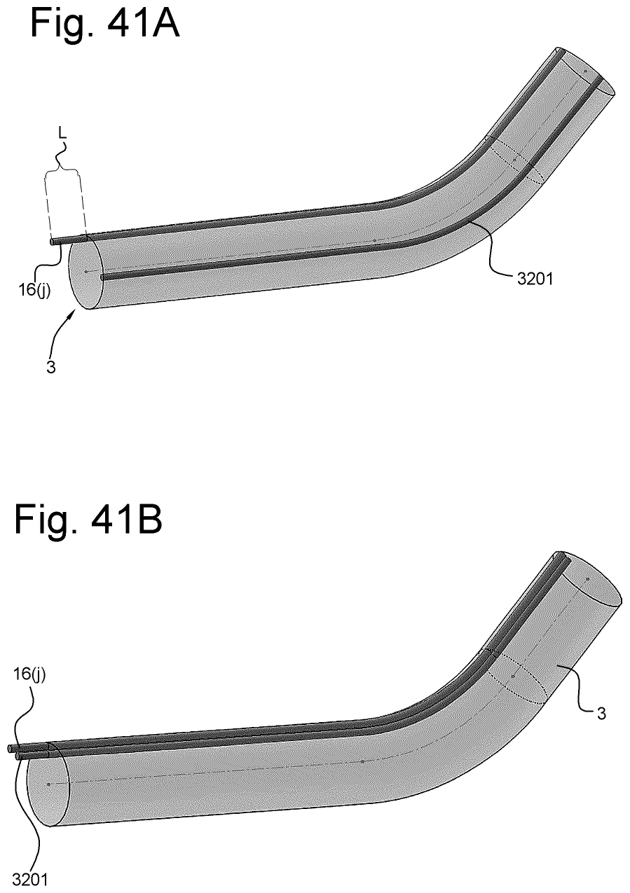

FIGS. 42A-42H show one embodiment of a length compensation section at a proximal end of a tubular steerable instrument in which all components result from making suitable slotted patterns in several coaxially arranged tubes and attaching several of the resulting components to other components in another, adjacent tube, e.g. by (laser) welding, gluing, etc.

FIG. 42A shows a first tube 4202 out of which the body length sensing elements and the steering wires are cut, FIGS. 42B, 42C, 42D, and 42E, respectively, show a second, third, fourth and fifth tube 4204, 4206, 4208, and 4210, respectively, placed on top of each other in that order in the finally assembled state.

FIG. 42A shows two steering wires 16(1), 16(2) of a total of four steering wires. At the proximal end of the instrument, first tube 4202 comprises four body length sensing elements 3201(1)-3202(4) of which two are visible. In the example of FIG. 42A adjacent sensing elements 3201 (i) touch one another along at least a portion of their length at the proximal end. Each sensing element 3201(i) has a smaller (less wide) sensing element portion extending towards the distal end between two adjacent steering wires 16(i), 16(i+1). At their distal end these smaller sensing element portions are attached to a bendable or flexible section of which bending would result in an undesired length change of one or more steering wires 16(i) (cf. FIG. 36).

FIG. 42B shows second tube 4204 on top of first tube 4202. This figure shows a first steering wire attachment 4212(1) that is attached, e.g. welded, to the end of steering wire 16(1) in first tube 4202 at one or more attachment locations 4214(1). It also shows a sense element attachment 4220(1) attached, e.g. welded, to the end of body length sense element 3201(1) of first tube 4202 at one or more attachment locations 4222(1). It also shows a ‘fixed world’ tubular member 4205 with a first opening 4216(1) for linear guiding of first steering wire attachment 4212(1) in the longitudinal direction and a second opening 4218(1) for linear guiding of body length sensing element attachment 4220(1) in the longitudinal direction.

Second tube 4204 also comprises first steering wire attachments 4212(i) attached to steering wire 16(i) and located inside openings 4216(i) for every other steering wire 16(i). Their functioning is the same as of first steering wire attachment 4212(1). Moreover, second tube 2404 comprises a sense element attachment 4220(i) attached to a respective sense element 3201 (i) and located in opening 4218(i) for every sense element 3201 (i). Their functioning is the same as of sense element attachment 4220(1).

FIG. 42C shows a ‘fixed world’ cylinder 4206(1) of third tube 4206 which is attached, e.g. welded, to the ‘fixed world’ tubular member 4205 of second tube 4204 at one or more attachment locations 4207. Second steering wire attachments 4224(i) are located inside an opening 4226(i) configured for guiding second steering wire attachment 4224(i) in the longitudinal direction. Every second steering wire attachment 4224(i) is attached to first steering wire attachment 4212(i) in second tube 4204 at one or more attachment locations 4228(i), e.g. by welding.

The figure also shows first and second length compensation activation cylinders 4206(2) and 4206(4) which may be separated by a short cylinder 4206(3). Their function is the same as the one of slider 3218 in FIGS. 32-40. They can rotate freely about second tube 4204 as indicated by arrows C and D. There is one activation cylinder for the left-right plane of the flexible body and one cylinder for the up-down plane.

First length compensation activation cylinder 4206(2) is provided with two slots 4234 extending at opposite angles (>0 but <90 degrees) relative to the circumferential direction. A protrusion (not visible in FIG. 42C) is provided inside each slot 4234 which is attached to the end of sensing element attachment 4220(i). These protrusions are comparable to the protrusions 3206 inside first slider 3218 of e.g. FIG. 32. Second length compensation activation cylinder 4206(4) is provided with two slots 4236 extending at opposite angles (>0 but <90 degrees) relative to the circumferential direction. A protrusion 4238(i), one of which is indicated with reference number 4238(1), is provided inside each slot 4236. Protrusion 4238(1) is attached to the end of sense element attachment 4220(1). These protrusions 4238(i) are also comparable to the protrusions 3206 inside first slider 3218 of e.g. FIG. 32.

Thus, FIG. 42C shows how two opposing sense elements 3201(2), 3201(4) are connected to one “first” slider 4206(2) and two other opposite sense elements 3201(1), 3201(3) (rotated 90 degrees relative to the first mentioned two) are connected to one other “first” slider 4206(4)

Rotation of first and second length compensation activation cylinders 4206(2) and 4206(4) is activated by protrusions 4238(i), i.e., they operate as cam followers. When a body length sensing element 3201(i) moves in a longitudinal direction, the respective cam follower forces the respective cylinder to rotate, cf. protrusions 3206 in slot 3212 in slider 3218.

FIG. 42D shows fourth tube 4208 on top of third tube 4206. Fourth tube 4208 comprises a ‘fixed world’ cylinder 4208(1) which is provided with openings 4240(i), one per steering wire 16(i). A third steering wire attachment 4242(i) is arranged inside each opening 4240(i) and attached to second steering wire attachment 4228(i) in third tube 4206. Openings 4240(i) are configured to guide third steering wire attachments 4242(i) linearly in the longitudinal direction. Fixed world cylinder 4208(1) is attached to fixed world cylinder 4206(1) at one or more attachment locations 4241, 4250.

Fourth tube 4208 also comprises a cylinder 4208(2) with two longitudinally extending strips 4248 which are located 180 degrees tangentially rotated relative to one another and act as linear guiding strips. Linear guiding strips 4248 are attached, e.g. welded, to length compensation cylinder 4206(2) (acting as slider 3218) such that they can rotate together with length compensation cylinder 4206(2). Between these two longitudinally extending strips 4248 fourth tube 4208 comprises two length compensation sliders 4208(3) (acting as sliders 3219) which are located 180 degrees tangentially rotated relative to one another. Each length compensation slider 4208(2) comprises two slots 4254, 4256 oriented at opposing angles to the circumferential direction (these slots are comparable to slots 3214, 3216 in the embodiment of FIG. 34). These slots 4254, 4256 accommodate respective protrusions (not visible in FIG. 42D but comparable to protrusions 3208, 3210) attached to respective steering wire portions (like 16(i, 1), 16(i,2) in FIG. 34). Cf. FIG. 42E how this can be implemented. The strips 4248 prevent that the length compensation sliders 4208(3) can move in a tangential/circumferential direction if length compensation cylinder 4206(2) does not rotate and they guide the length compensation sliders 4208(3) in the longitudinal direction. Therefore, if length compensation cylinder 4206(2) rotates due to longitudinal displacement of a body length sensing element 3201 (i), length compensation sliders 4208(3) (like slider 3219) are forced to move in the longitudinal direction.

Fourth tube 4208 also comprises a cylinder 4208(5) with two longitudinally extending strips 4265, 4267 which are located 180 degrees tangentially rotated relative to one another and act as linear guiding strips. Linear guiding strips 4265, 4267 are attached, e.g. welded, to length compensation cylinder 4206(2) (acting as slider 3218) such that they can rotate together with length compensation cylinder 4206(4). Between these two longitudinally extending strips 4265, 4267 fourth tube 4208 comprises two length compensation sliders 4208(4) (acting as sliders 3219) which are located 180 degrees tangentially rotated relative to one another. Each length compensation slider 4208(4) comprises two slots 4258, 4260 oriented at opposing angles to the circumferential direction (these slots are comparable to slots 3214, 3216 in the embodiment of FIG. 34). These slots 4258, 4260 accommodate respective protrusions 4262, 4264 (comparable to protrusions 3208, 3210) attached to respective steering wire portions (like 16(i,1), 16(i,2) in FIG. 34). Cf. FIG. 42E how this can be implemented. The strips 4265, 4267 prevent that the length compensation sliders 4208(4) can move in a tangential/circumferential direction if length compensation cylinder 4206(4) does not rotate and they guide the length compensation sliders 4208(4) in the longitudinal direction. On the other hand, if length compensation cylinder 4206(4) rotates due to longitudinal displacement of a body length sensing element 3201 (i), length compensation sliders 4208(4) (like slider 3219) are forced to move in the longitudinal direction.

FIG. 42E shows fifth tube 4210. This fifth tube 4210 comprises a next ‘fixed world’ cylinder 4277 with linear guidings 4278(i). Between two adjacent linear guidings 4278(i), 4278(i+1) fifth tube 4210 has a longitudinal opening 4268 accommodating a first portion 4270(i) (comparable to 16(i,1)) and a second portion 4274(i) (comparable to 16(i,2) of a steering wire 16(i). Fixed world cylinder 4277 with linear guidings 4278(i) is attached to the underlying fixed world cylinder 4208(1), e.g. by laser welding, at one or more attachment locations 4276, 4280.

First steering wire portion 4270(i) (steering wire portion 16(i, 1)) can only move in the longitudinal direction and the linear guidings 4278(i) prevent movement in a tangential/circumferential direction. The same applies to the second steering wire portion 4274(i) (steering wire portion 16(i,2)).

At its distal end, first steering wire portion 4270(i) is attached to third attachment slider 4242(i) at an attachment location 4272, e.g. by laser welding. At its proximal end, first steering wire portion 4270(i) is attached to protrusion 4262 in slot 4258 at an attachment location 4273. At its distal end, second steering wire portion 4274(i) is attached to protrusion 4264 in slot 4260 at an attachment location 4275, e.g. by laser welding. Therefore if length compensation slider 4206(4) rotates, forced by longitudinal displacement of a body length sensing element, and one holds the proximal steering wire 16(i) in a fixed position, linear guidings 42645, 4267 will also rotate together with length compensation sliders 4208(4) and the protrusions 4262, 4264 will force the first steering wire portion 4270(i) in a required longitudinal direction over the required distance for full length compensation of steering wire 16(i).

FIGS. 42F-42H show an embodiment in which protrusions 4262 and 4264 are not applied but substituted by inwardly bent lips. Here, at its proximal end, first steering wire portion 4270(i) is provided with a lip 4282 bent inwardly in slot 4258. Moreover at its distal end, second steering wire portion 4274(i) is provided with a lip 4284 bent inwardly in slot 4260. FIG. 42H shows lip 4284 on an enlarged scale.

It will be evident to persons skilled in the art how the mechanism works for all four steering wires 16(i) of the embodiment of FIGS. 42A-42H. Moreover, the mechanism also works for any other number of steering wires and number of sensing elements (which numbers do not need to be equal).

In practice, there is a last tubular element provided over the assembly shown in FIG. 42E, that protects the mechanism and radially holds the first steering wire portion 4270(i) and the second (proximal) steering wire portion 4274(i) in place. This tubular element is not drawn. Moreover, in practice an inner protective tubular element will be inserted into first tube 4202.

Obviously, the embodiment described above with reference to FIGS. 42A-42H is just one possible practical implementation of the principal mechanisms as proposed per FIGS. 32-41B. Within the scope of the invention, many more possible practical implementations are thinkable. One could build an instrument with less or more tubular elements than is described in the embodiment and in which the elements such as sliders, steering wires and sensing wires are arranged in different tubular elements than described in the embodiment. Of course, one can also envision, within the scope of this invention, that one could build an instrument where the steering wires are located on top of the body length sensing elements, or visa versa, in a different tubular element. One could also envision an instrument with more than one length compensation slider (slider 3218) and that separate steering wire bridging elements can connect these sliders as in FIG. 35. One could also envision a tubular instrument with more than one group of body length sensing elements, each with its own length compensation mechanism as in FIG. 36. One can also envision an instrument with two or more steerable sections in the tip of the instrument and that a mechanism with one body length sensing wire input can compensate the length of two or more sets of steering wires simultaneously as in FIG. 39. Also other combinations of different aspects of the mechanisms as shown in FIGS. 32-41B are possible, one can for example envision an instrument with two sets of body length sensing wires as in FIG. 37 combined with two sets of steering wires as in FIG. 39. Etc.

### Fracture Elements

In the above described steerable instrument, during manufacturing, fracture elements are applied. Examples of fracture elements and the way they can be used during the manufacturing were first explained in detail in WO2016/089202 of the present applicant.

In general, as e.g. explained here with reference to fracture elements 1629(j), such fracture elements can be designed in the following way. Fracture elements are made in the same process step as other elements are cut from a tube. Before being fractured, each fracture element is attached to opposite portions of two tube portions. In this way they keep these two opposite portions together and prevent the two portions from falling apart after the cutting process. These opposite portions have a geometrical shape such that the stresses in the fracture element will increase more than the stresses in the surrounding material and/or structure during manipulation. Therefore, if a deflection or a high enough force is applied on the two opposite portions such as to try to move them relative to one another, the stress in the fracture element rises above the yield stress of the tube material, causing permanent deflection of the fracture element. Applying even more deflection or a higher force results in the stress reaching the ultimate tensile stress of the fracture element causing a fracture of fracture element without causing a permanent deformation of the two portions because the stress as developed in the two portions remains below their yield stress.

In this way elements of the tubes that should be independently movable relative to one another in the final steerable instrument can be separated while the different tubes are inserted into one another and the elements cannot fall apart anymore. I.e., the process of fracturing is preferably done when the steerable instrument is finished and all tubes are inserted into one another, and the elements that should be attached to one another have been attached.

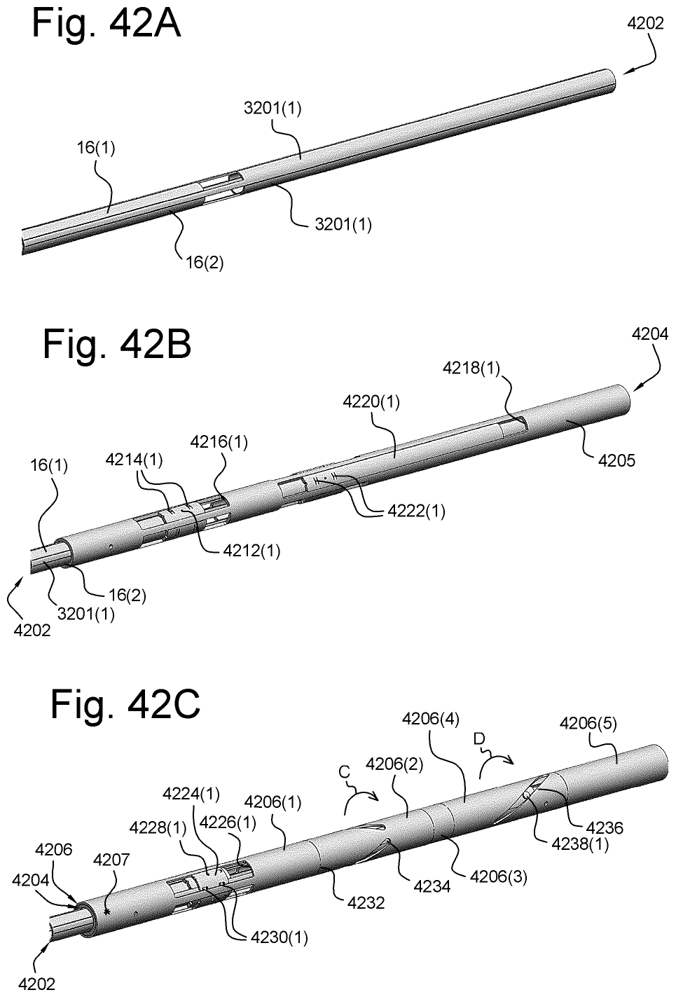

This is schematically shown in FIGS. 43, 44 and 45. FIG. 43 shows a fracture element 4306 attached to a first tube portion 4302 and a second tube portion 4304 of a tube 4300. Here, fracture element 4306 has the form of a small disk attached to first and second tube portions 4302, 4304 via small bridges. In FIG. 43 first and second tube portions 4302 and 4304 can move relative to one another in the longitudinal direction of the tube 4300. The bridges of fracture element 4306 to the opposite first and second portions 4302 and 4304 will fracture once they move relative to one another and the above stress conditions apply.

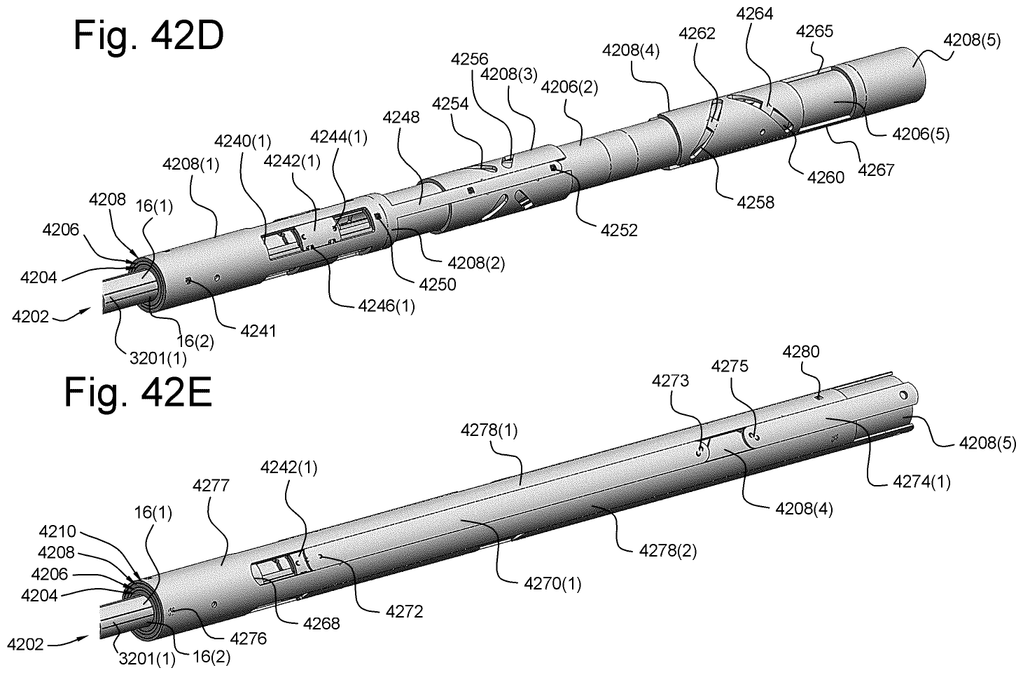

FIG. 44 shows an embodiment with two opposite first and second tube portions 4302, 4304 which, during assembly, remain attached to one another by fracture element 4306 in the form of a small bridge. In this embodiment, opposite first and second tube element 4302, 4304 can rotate relative to one another in the surface of the drawing as indicated with arrow 4402. Once they rotate relative to one another forces are developed inside fracture element 4306 and inside the surrounding material of the opposite tube elements 4302, 4304 until a moment fracture element 4306 fractures because the stress inside fracture element 4306 rises above the ultimate tensile stress, as explained above.

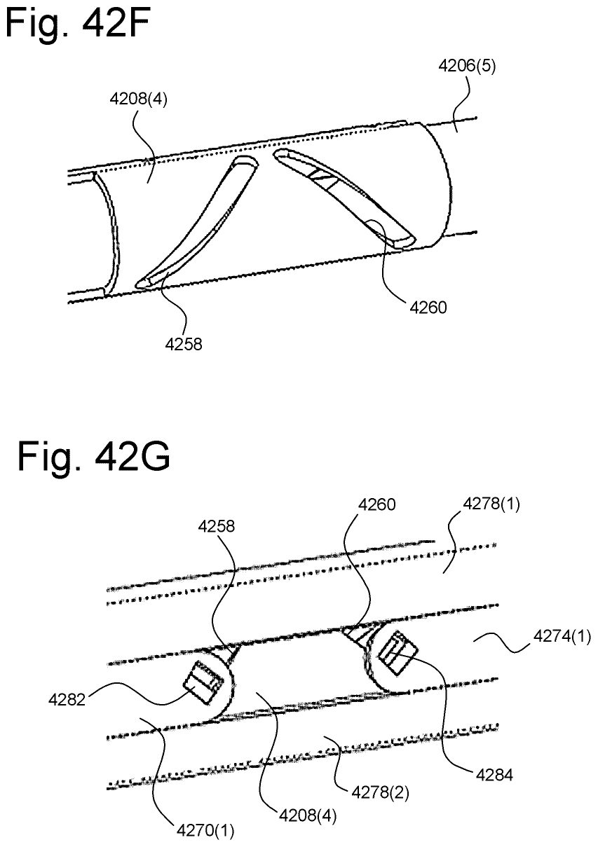

FIG. 45 shows an alternative to the one shown in FIG. 44 in which first and second tube portions 4302, 4304 can rotate relative to one another as indicated with an arrow 4502. Now, fracture element 4306 has the shape of a small disk attached to the two opposite tube portions 4302, 4304 by means of small bridges. In this embodiment these bridges will fracture under the above explained stress conditions.

Although FIGS. 43, 44 and 45 show the application of fracture elements 4306 between two opposite tube portions 4302, 4304 that can move in the longitudinal direction relative to one another or rotate relative to one another, they can be used everywhere in the steerable instrument between two opposite tube portions that move relative to one another in usage of the steerable instrument, be it rotational, longitudinal, radial or tangential, because a large enough movement during use will eventually fracture these fracture elements 4306.

An other mechanism to break the fracture element 4306 may be achieved by applying low or high cycle fatigue to a fracture element. The stress in fracture element is raised above the fatigue limit, causing a fatigue fracture. Note that this fatigue limit is lower than the above mentioned ultimate tensile stress. In all cases the stresses in the surrounding structure/material of the two opposite tube elements to which fracture element 4306 is attached stays at least below the yield stress of the tube material. The process of fracturing by applying several fatigue cycles is preferably done when the steerable instrument is finished and all tubes are inserted into one another, and the elements that should be attached to one another have been attached

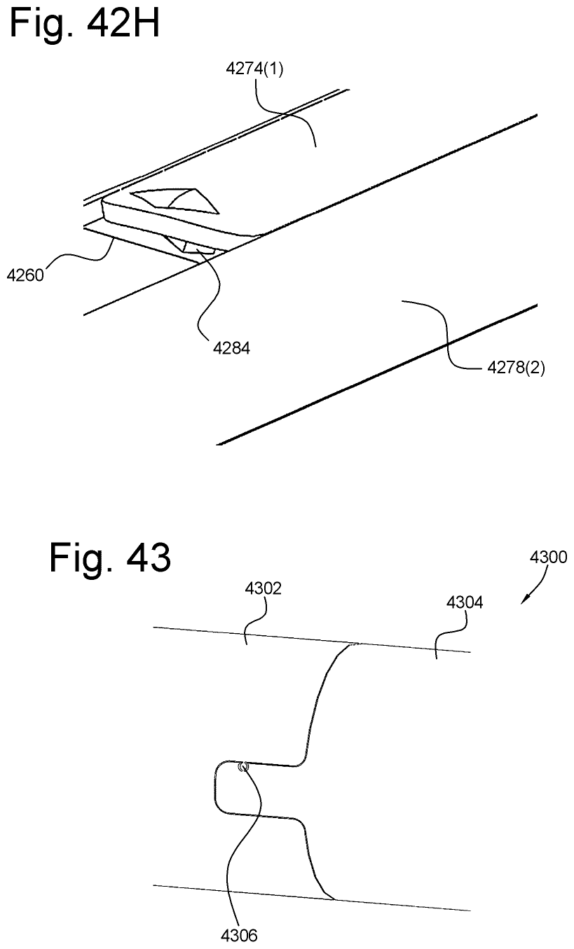

FIGS. 46-49 show alternative structures to fracture elements, i.e., melt elements.

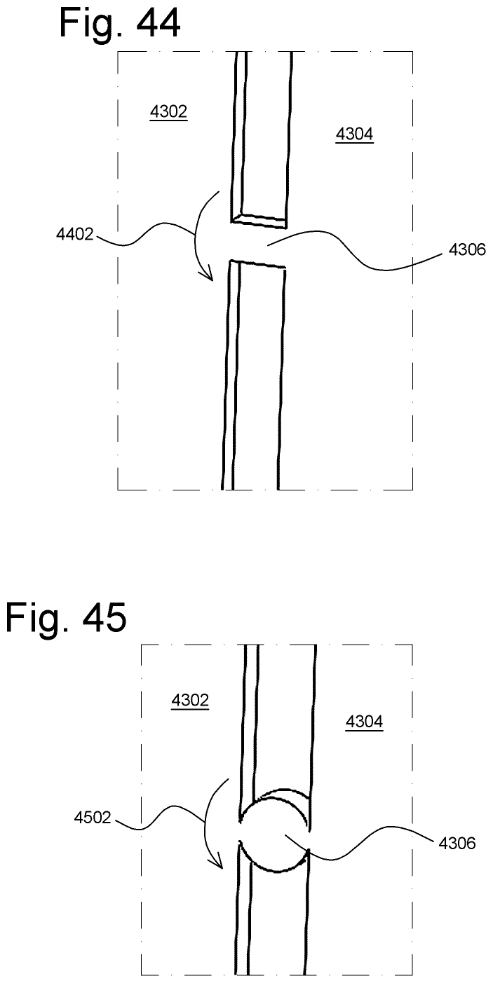

FIG. 46 shows an example of a melt element with a larger portion 4606 which after the cutting process is attached to second tube portion 4304 and a small bridge 4604 attached to first tube portion 4306. Here, first and second tube portions 4302, 4304 are intended to rotate relative to one another during normal usage of the steerable instrument. Now, the melt element 4604/4606 is irradiated with an energy beam, e.g., a laser beam, such that it melts and the melted material of larger portion 4606 attaches second tube element 4304 to a tube portion of a tube inside the shown tube. At the same time, the attachment between the first and second tube portions 4302, 4304 is released because the small bridge 4604 of the melt element disconnects.

Whereas FIG. 46 shows an embodiment in which melt element has a round larger portion 4606 attached to second tube portion 4304, FIG. 47 shows an embodiment in which larger portion 4606 has a rectangular shape. Other shapes are also possible without deviating from the inventive concept.

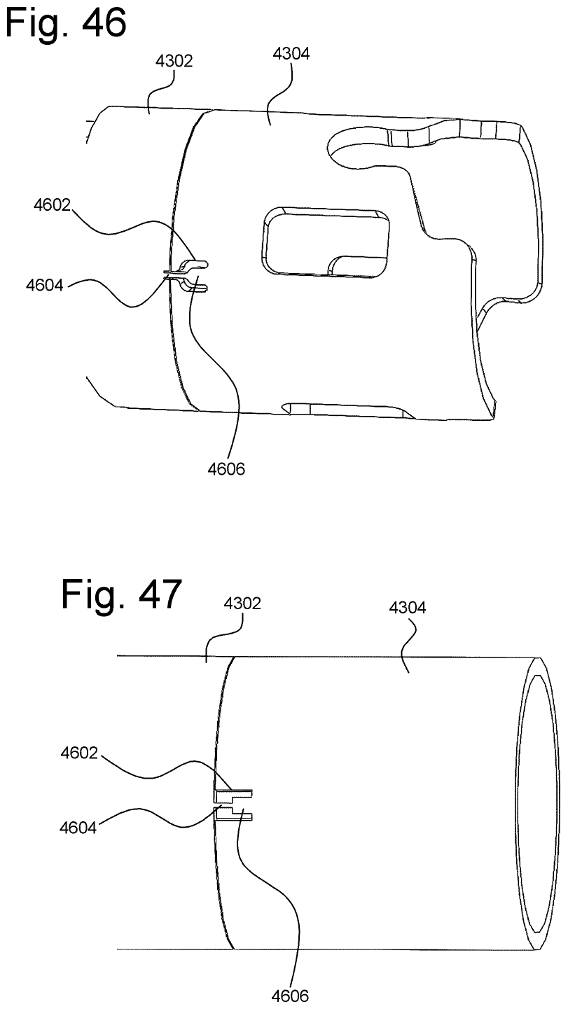

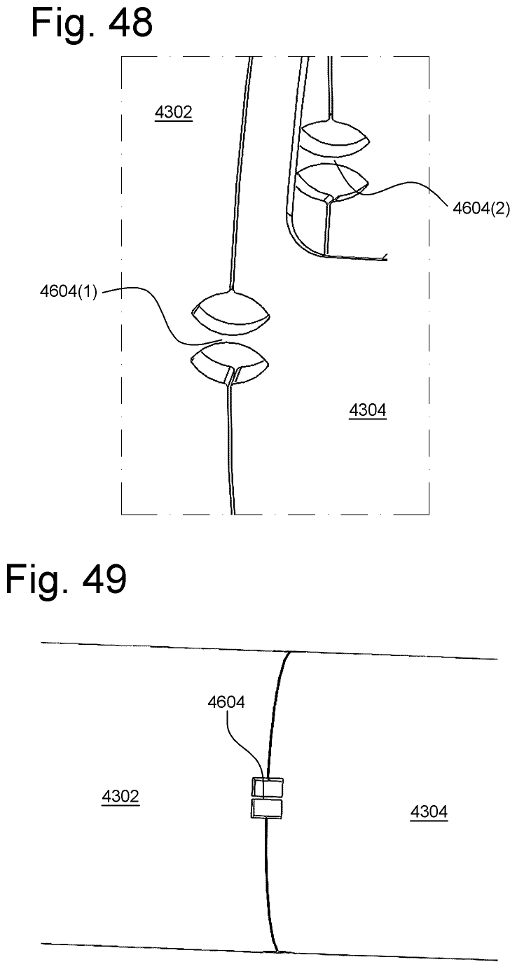

FIGS. 48, 49 show melt elements only having a smaller bridge 4604(1), 4604(2), 4604 with different possible shapes. Also other shapes may be applied. To release the attachment between first and second tube elements 4302, 4304, again an energy beam, e.g., a laser beam, is directed to the smaller bridge 4604(1), 4604(2), 4604 such that it evaporates.

Although FIGS. 46-49 show the application of melt elements 4604/4606 between two opposite tube portions 4302, 4304 that are intended to rotate relative to one another in use, they can be used everywhere in the steerable instrument between two opposite tube portions that move relative to one another in usage of the steerable instrument, be it rotational, longitudinal, radial or tangential.

It is observed that a melt element can also be designed to release an attachment between two tube portions by applying several steps, i.e., a first step in which the melt element is only partly evaporated and a second step in which the remaining part of the melt element is fractured either by the above explained fracturing process or the process of applying several fatigue cycles.

The melting process is performed at any suitable moment during manufacturing of the steerable instrument, as long as the melt element can be reached by the energy beam, e.g. via a suitable opening in a surrounding tube.

The fracture elements and melt elements as explained with reference to FIGS. 43-49 can be applied in any of tubes of any of the steerable instruments explained with reference to FIGS. 1-42.

### General Statements

The material removal means can be a laser beam that melts and evaporates material or water jet cutting beam and this beam can have a width of 0.01 to 2.00 mm, more typically for this application, between 0.015 and 0.04 mm. So slots between adjacent parts of a tube may have a minimum width of between 0.01-2.00 mm, more specifically 0,015-0.04 mm.

The wall thickness of tubes depend on their application. For medical applications the wall thickness may be in a range of 0.03-2.0 mm, preferably 0.03-1.0 mm, more preferably 0.05-0.5 mm, and most preferably 0.08-0.4 mm. The diameter of tubes depend on their application. For medical applications the diameter may be in a range of 0.5-20 mm, preferably 0.5-10 mm, more preferably 0.5-6 mm. The radial play between adjacent tubes may be in range of 0.01-0.3 mm.

Longitudinal and other elements in one tube can be attached to longitudinal and other elements in adjacent tubes such that they are together operable to transfer a longitudinal motion from a steering wire at the proximal end of the instrument to a bendable portion of the instrument at the distal end of the instrument such that the bendable portion bends. This is explained in detail in WO 2017/213491 (cf. e.g. FIGS. 12, 13a and 13b in that PCT application) of the present applicant.

It will be clear to a person skilled in the art that the scope of the invention is not limited to the examples discussed in the foregoing, but that several amendments and modifications thereof are possible without deviating from the scope of the invention as defined in the attached claims. While the invention has been illustrated and described in detail in the figures and the description, such illustration and description are to be considered illustrative or exemplary only, and not restrictive. The present invention is not limited to the disclosed embodiments but comprises any combination of the disclosed embodiments that can come to an advantage.

Variations to the disclosed embodiments can be understood and effected by a person skilled in the art in practicing the claimed invention, from a study of the figures, the description and the attached claims. In the description and claims, the word “comprising” does not exclude other elements, and the indefinite article “a” or “an” does not exclude a plurality. In fact it is to be construed as meaning “at least one”. The mere fact that certain features are recited in mutually different dependent claims does not indicate that a combination of these features cannot be used to advantage. Any reference signs in the claims should not be construed as limiting the scope of the invention. Features of the above described embodiments and aspects can be combined unless their combining results in evident technical conflicts.

1. A cylindrical instrument comprising at least a first tube, a second tube surrounding the first tube, and a third tube surrounding the second tube, the instrument having at least one deflectable tip section, a steering section, a flexible body section between the tip section and the steering section, a length compensation section, and one or more steering wires extending from the steering section to the tip section such that the tip section can be deflected by moving the one or more steering wires in a longitudinal direction of the cylindrical instrument, wherein the length compensation section comprises a first portion of a steering wire at least partly surrounded by steering wire guiding portions, both each steering wire and associated steering wire guiding portions being portions of at least one of the first tube, the second tube, or the third tube, and each first portion of a steering wire and associated steering wire guiding portions being configured as a length compensation element which has a curved configuration which is configured to deform to absorb a length change of a second portion of the steering wire inside the flexible body section due to bending of the flexible body section.

2. The cylindrical instrument according to claim 1, wherein, the curved configuration is at least one of extending radially outward from a central axis, extending radially inward towards the central axis, extending tangentially relative to the central axis, or spiraling about a central axis of the instrument.

3. The cylindrical instrument according to claim 1, wherein the steering wire guiding portions comprise at least one longitudinal guiding element length compensation portion alongside the first portion of each steering wire, the at least one longitudinal guiding element length compensation portion being a portion of the same at least one of the first tube, the second tube, or the third tube of which the steering wire is a portion.

4. The cylindrical instrument according to claim 3, wherein the at least one longitudinal guiding element length compensation portion comprises two longitudinal guiding element length compensation portions, along different sides of the first portion of each steering wire.

5. The cylindrical instrument according to claim 1, wherein, in the length compensation section, steering wire guiding portions associated with one steering wire comprise an inner steering wire guiding portion which is a portion from the first tube and an outer steering wire guiding portion which is a portion from the third tube.

6. The cylindrical instrument according to claim 5, wherein, in the length compensation section, steering wire guiding portions comprise one or more radially oriented lips located tangentially adjacent to the steering wire and attached to at least one of the inner steering wire guiding portion or the outer steering wire guiding portion.

7. The cylindrical instrument according to claim 5, wherein, in the length compensation section, steering wire guiding portions comprise one or more islands which are slidably arranged in at least one steering wire such as to limit tangential movement of the at least one steering wire.

8. The cylindrical instrument according to claim 1, wherein a square element is provided which has a first bar and a second bar (both extending in a first direction and a third bar and a fourth bar both extending in a second direction perpendicular to the first direction, the first and second bars being slidably connected to apexes of opposite sets of one steering wire and associated steering wire guiding portions in the length compensation section, the third and fourth bars being slidably connected to apexes of other opposite sets of a steering wire and associated steering wire guiding portions in the length compensation section.

9. The cylindrical instrument according to claim 1, wherein portions of one steering wire are located in different ones of the first, second and third tubes in at least two different sections from the at least one deflectable tip section, the steering section, the flexible body section, and the length compensation section.

10. A cylindrical instrument comprising at least one deflectable tip section, a steering section, a flexible body section between the tip section and the steering section, a length compensation section, and one or more steering wires, extending from the steering section to the tip section such that the tip section can be deflected by moving the one or more steering wires in a longitudinal direction of the longitudinal instrument, wherein the cylindrical instrument comprises a Bowden cable arrangement for each steering wire inside the body section and the length compensation section, each Bowden cable arrangement comprising at least one steering wire and a steering wire guiding, each steering wire guiding having a distal steering wire guiding end attached to the cylindrical instrument at the transition between the tip section and the body section, and a proximal steering wire guiding end extending inside the length compensation section, the cylindrical instrument having one or more sensors, each sensor being configured to sense longitudinal movement of one of the proximal steering wire guiding ends, one or more actuators each actuator being configured to control longitudinal movement of one steering wire such as to compensate longitudinal movement of an associated proximal steering wire guiding end due to bending of the body section.

11. The cylindrical element according to claim 10, wherein each sensor is configured to send a sensor signal to a processor, the processor being configured to generate an actuation signal for each actuator in dependence on an associated sensor signal such that each actuator performs compensated longitudinal movement of the associated steering wire.

12. The cylindrical element according to claim 10, wherein each sensor is implemented as a mechanical coupling such that each actuator is mechanically coupled to an associated proximal steering wire guiding end and is configured to move with the proximal steering wire guiding end during body section bending and to keep a distance that the steering wire is extending proximally outside an associated steering wire guiding constant as long as no actuation signal is received by the actuator.

13. The cylindrical instrument according to claim 10, comprising a brake device for each steering wire guiding proximal end, each brake device being configured to either allow or block longitudinal movement of the respective proximal steering wire guiding end.

14. A cylindrical instrument comprising at least a first tube, a second tube surrounding the first tube, and a third tube surrounding the second tube, the instrument having at least one deflectable tip section, a steering section, a flexible body section between the tip section and the steering section, a length compensation section, and one or more steering wires extending from the steering section to the tip section such that the tip section can be deflected by moving the one or more steering wires in a longitudinal direction of the cylindrical instrument, each steering wire comprising portions of at least one of the first tube, the second tube, or the third tube, wherein, in the length compensation section, each steering wire comprises a first portion and a second portion, the first portion comprising a protrusion and the second portion comprises a recess receiving the protrusion, both the protrusion and the recess being angled relative to a tangential direction of the cylindrical instrument, and configured to compensate length changes of the one or more steering wires in the body section due to bending of the body section.

15. A cylindrical steerable instrument having at least one deflectable tip section, a steering section, at least one flexible body section located between the tip section and the steering section, a length compensation section located at a location between the at least one flexible body section and the steering section, and one or more steering wires extending from the steering section to the deflectable tip section such that the tip section can be deflected by moving the one or more steering wires in a longitudinal direction of the cylindrical steerable instrument, comprising:
for at least one steering wire, one or more sensing elements having a first end attached to the at least one flexible section and a second end extending into the length compensation section, and configured to move in the longitudinal direction in the at least one flexible section due to bending of the at least one flexible section and then to cause a first slider to move in a tangential direction of the cylindrical steerable instrument,
at least one second slider, each second slider being configured to move in the tangential direction together with one first slider but to move in a longitudinal direction of the cylindrical steerable instrument independent from the one first slider;
the at least one steering wire comprising a first steering wire portion and a second steering wire portion, the first steering wire portion and the second steering wire portion being both connected to the at least one second slider such that if the at least one second slider moves in the tangential direction a mutual longitudinal distance between the first steering wire portion and the second steering wire portion is adjusted.

16. A method of manufacturing a cylindrical steerable instrument comprising
providing at least a first tube;
making a slotted pattern in the at least first tube such as to generate a first tube portion a second tube portion which are attached to one another by means of at least one fracture element; and
releasing the attachment between the first tube portion and the second tube portion by applying several fatigue cycles to the at least one fracture element.

17. A method of manufacturing a cylindrical steerable instrument comprising
providing at least a first tube;
making a slotted pattern in the at least first tube such as to generate a first tube portion a second tube portion which are attached to one another by means of at least one melt element; and
releasing the attachment between the first tube portion and the second tube portion by directing an energy beam, such as a laser beam, to the melt element.

18. The method according to claim 17, comprising attaching at least one of the first tube portion or the second tube portion by a melted portion of the melt element to a portion of a second tube inside the first tube.
